(19) United States
(12) Patent Application Publication
(10) Pub. No.: US 2025/0170117 A1
(43) Pub. Date: May 29, 2025

(54) MODULATORS OF ACKR3 AND USES THEREOF

(71) Applicants: InterAx Biotech AG, Villigen (CH); Stichting VU, Amsterdam (NL)

(72) Inventors: Rick RIEMENS, Amsterdam (NL); Reggie BOSMA, Amsterdam (NL); Rob LEURS, Amsterdam (NL); Iwan DE ESCH, Amsterdam (NL); Maikel WIJTMANS, Amsterdam (NL); Henry VISCHER, Amsterdam (NL); Yaroslav NIKOLAEV, Villigen (CH); Georgi KANEV, Villigen (CH); Mirjam ZIMMERMANN, Villigen (CH); Aurélien RIZK, Villigen (CH); Alessandro POTENZA, Villigen (CH); Tomasz Maciej STEPNIEWSKI, Villigen (CH); Laura WIJFFELAARS, Amsterdam (NL)

(21) Appl. No.: 18/843,308

(22) PCT Filed: Mar. 3, 2023
(86) PCT No.: PCT/EP2023/055469

§ 371 Date: Sep. 3, 2024

(30) Foreign Application Priority Data
Mar. 3, 2022 (EP) 22159896.4

(51) Int. Cl. A61K 31/454; C07D 413/12; C07D 417/12

(52) U.S. Cl. CPC A61K 31/454; C07D 413/12; C07D 417/12

(57) ABSTRACT

The present disclosure relates to compounds of formula (I) comprising a (4-piperidinylmethyl) carboxamide moiety, which can act as modulators of ACKR3. The present disclosure also relates to the use of these compounds as a drug.

### TECHNICAL FIELD

The present disclosure relates to compounds of formula (I) comprising a (4-piperidinylmethyl) carboxamide moiety, which can act as modulators of ACKR3. The present disclosure also relates to the use of these compounds as a drug.

### BACKGROUND ART

Chemokines and G protein-coupled chemokine receptors (GPCRs) play an important role in the immune defense system by controlling the migration, activation, differentiation, and survival of leukocytes. Endogeneous chemokine proteins stabilize their cognant chemokine receptors in an active conformation that facilitates intracellular signal transduction by interactions with G proteins and/or arrestins. Because of their crucial role in the migration of immune cells, chemokine receptors are promising drug targets for various immune-related diseases, including chronic obstructive pulmonary disease, multiple sclerosis, rheumatoid arthritis, HIV-1 infection and cancer. Molecular pharmacology, medicinal chemistry, and molecular modeling studies have provided insights into molecular determinants of chemokine receptor modulation by proteins, peptides, and small-molecule ligands.

The chemokine receptor ACKR3, formerly called CXCR7, is activated by the chemokine peptides and majorly by chemokine CXCL12. The receptor is overexpressed in many cancer types indicating a vital role in the development of the disease. In vitro investigation of the ACKR3 receptor showed that overexpression and activation of the receptor by CXCL12 leads to oncogenic events such as angiogenesis, metastasis and trans endothelial cell-migration. ACKR3 knock-out models or ACKR3 inhibition by small-molecule ligands led to reduced oncogenic effects. Recently, the ACKR3 has been claimed to scavenge a broad range of opioid peptides, which might lead to a broadened application of a potential drug.

However, to date, no drug has been developed targeting the ACKR3 receptor. Thus, in this context, it is desirable to develop molecules that can act as modulators of ACKR3. It is also desirable to develop molecules that can act as modulators of ACKR3, while being easy to synthesize.

### SUMMARY OF THE DISCLOSURE

The inventors surprisingly found that some compounds comprising a (4-piperidinylmethyl) carboxamide moiety can act as modulators of ACKR3.

Consequently, in a first aspect, the present disclosure relates to a compound formula (I):

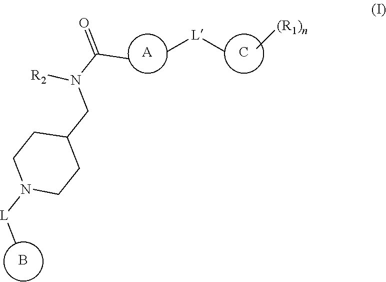

Wherein
Each R₁ is independently selected from halogen;
n is an integer between 0 and 5, preferably between 1 and 5;
Ring A is an heteroaryl having 5 ring atoms;
L′ is a bond or a divalent linker selected from the group consisting of C₁-C₆ alkylene, —O—, and C₁-C₆ heteroalkylene;
Ring C is a ring selected from the group consisting of C₃-C₁₂ cycloalkyl, heterocyclyl having 5 to 10 ring atoms, aryl having 6 to 10 ring atoms, and heteroaryl having 5 to 10 ring atoms;
R₂ is selected from H, C₁-C₆ alkyl, C₃-C₆ cycloalkyl, heterocyclyl having 5 to 10 ring atoms, aryl having 6 to 10 ring atoms, and heteroaryl having 5 to 10 ring atoms; said alkyl, cycloalkyl, heterocyclyl, aryl and heteroaryl being optionally substituted with one or more substituents independently selected from oxo, halogen, —OH, —NO₂, —CN, and C₁-C₆ alkyl, preferably R2 is H;
L is a bond or a divalent linker selected from the group consisting of —(CR₃R₄)ₚ— p being an integer between 1 and 12, C₁-C₁₂ heteroalkylene, C₃-C₆ cycloalkylene, C₂-C₁₂ alkenylene, C₂-C₁₂ alkynylene, —O—, —NR′—, —S—, —SO—, and —SO₂—, said heteroalkylene, alkenylene, and alkynylene being optionally interspersed with one or more groups selected from —(C₃-C₆ cycloalkyl)-, and said alkylene, heteroalkylene, cycloalkylene, alkenylene, and alkynylene being optionally substituted with one or more substituents independently selected from oxo, halogen, —OH, —NO₂, —CN, C₁-C₆ alkyl, C₃-C₆ cycloalkyl, heterocyclyl having 5 to 10 ring atoms, aryl having 6 to 10 ring atoms, heteroaryl having 5 to 10 ring atoms, C₁-C₆ alkoxy, C₁-C₆ haloalkyl, C₁-C₆ haloalkoxy, —(CO)—R′, —O—(CO)—R′, —(CO)—O—R′, —(CO)—NR″R″′, —NR″—(CO)—R′, and —NR″R″′;
Ring B is a ring selected from the group consisting of C₃-C₁₂ cycloalkyl, heterocyclyl having 5 to 10 ring atoms, aryl having 6 to 10 ring atoms, and heteroaryl having 5 to 10 ring atoms, said cycloalkyl, heterocyclyl, aryl and heteroaryl being optionally substituted with one or more substituents independently selected from oxo, halogen, —OH, —NO₂, —CN, C₁-C₆ alkyl, C₃-C₆ cycloalkyl, heterocyclyl having 5 to 10 ring atoms, aryl having 6 to 10 ring atoms, heteroaryl having 5 to 10 ring atoms, C₁-C₆ alkoxy, C₁-C₆ haloalkyl, C₁-C₆ haloalkoxy, —(CO)—R′, —O—(CO)—R′, —(CO)—O—R′, —(CO)—NR″R″′, —NR″—(CO)—R′, —NR″R″′, —S(O)₂-R′, and S(O)₂—NR″R″′;
R₃ and R₄ are independently selected from H, C₁-C₆ alkyl, C₃-C₆ cycloalkyl, heterocyclyl having 5 to 10 ring atoms, aryl having 6 to 10 ring atoms, heteroaryl having 5 to 10 ring atoms; said alkyl, cycloalkyl, heterocyclyl, aryl and heteroaryl being optionally substituted with one or more substituents independently selected from oxo, halogen, —OH, —NO₂, —CN, and C₁-C₆ alkyl;
Or R₃ and R₄, together with the C atom to which they are bonded, form a C₃-C₁₂ cycloalkyl or a heterocyclyl having 5 to 10 ring atoms;
R′ is selected from H, C₁-C₆ alkyl, C₃-C₆ cycloalkyl, heterocyclyl having 5 to 10 ring atoms, aryl having 6 to 10 ring atoms, and heteroaryl having 5 to 10 ring atoms;
R″ and R″′ are independently selected from H, C₁-C₆ alkyl, C₃-C₆ cycloalkyl, heterocyclyl having 5 to ring atoms, aryl having 6 to 10 ring atoms, heteroaryl having 5 to 10 ring atoms; said alkyl, cycloalkyl, heterocyclyl, aryl and heteroaryl being optionally substituted with one or more substituents independently selected from oxo, halogen, —OH, —NO₂, —CN, and C₁-C₆ alkyl;
Or R″ and R″′, together with the N atom to which they are linked, form a heterocycle having 5 to 10 ring atoms, or a heteroaryl having 5 to 10 ring atoms, said heterocyclyl, and heteroaryl being optionally substituted with one or more substituents independently selected from oxo, halogen, —OH, —NO₂, —CN, and C₁-C₆ alkyl;

and pharmaceutically acceptable salts, solvates and esters thereof.

In a second aspect, the disclosure relates to a pharmaceutical composition comprising a compound of the present disclosure and a pharmaceutically acceptable carrier.

In a third aspect, the disclosure relates to a compound of the present disclosure for use as a drug.

In a fourth aspect, the disclosure relates to a compound of the present disclosure for use in treating cancer, autoimmune disorders, inflammatory diseases, transplant rejection, fibrosis, or pain.

### DETAILED DESCRIPTION

### Definitions

As used herein, the terms “Cr-Cy alkyl”, by itself or as part of another substituent, refer to a linear or branched alkyl functional group having 1 to 12 carbon atoms, more preferably 1 to 6 carbon atoms. Suitable alkyl groups include methyl, ethyl, n-propyl, i-propyl, n-butyl, i-butyl, s-butyl and t-butyl, pentyl and its isomers (e.g. n-pentyl, iso-pentyl), and hexyl and its isomers (e.g. n-hexyl, iso-hexyl).

As used herein, the terms “C₃-C₁₂ cycloalkyl” refer to a saturated or unsaturated cyclic group having 3 to 12 carbon atoms, preferably 3 to 6. The cycloalkyl can have a single ring or multiple rings fused together. The cycloalkyl can also include spirocyclic rings. Suitable cycloalkyl groups include cyclopropyl, cyclobutyl, cyclopentyl and cyclohexyl.

As used herein, the term “halogen” refers to a fluoro (—F), chloro (—CI), bromo (—Br), or iodo (—I) group.

As used herein, the terms “C₁-C₆ haloalkyl” refer to a C₁-C₆ alkyl as defined herein that is substituted by one or more halogen group as defined herein. Suitable C₁-C₆ haloalkyl groups include trifluoromethyl and dichloromethyl.

As used herein, the terms “C₁-Cₓ heteroalkyl”, refer to a straight or branched hydrocarbon chain consisting of 1 to 12 carbon atoms, more preferably 1 to 6 carbon atoms, and from one to three, heteroatoms selected from the group consisting of O, N, Si and S, and wherein the nitrogen and sulfur atoms may optionally be oxidized (for example: a sulfoxide or a sulfone) and the nitrogen heteroatom may optionally be quaternized. The heteroatom(s) O, N and S may be placed at any interior position of the heteroalkyl group or at the position at which the alkyl group is attached to the remainder of the molecule.

As used herein, the terms “C₁-C₆ alkoxy” refer to a —O-alkyl group, wherein the alkyl group is a C₁-C₆ alkyl as defined herein. Suitable C₁-C₆ alkoxy groups include methoxy, ethoxy, propoxy.

As used herein, the terms “C₁-C₆ haloalkoxy” refer to a C₁-C₆ alkoxy group as defined herein, that is substituted by one or more halogen group as defined herein. Suitable haloalkoxy include trifluoromethoxy.

As used herein, the terms “C₁-Cₓ alkylene”, used alone or as part of another substituent, refer to a divalent saturated, straight-chained or branched hydrocarbon group having 1 to 12 carbon atoms, preferably 1 to 6.

As used herein, the terms “C₁-Cₓ heteroalkylene”, refer to a divalent heteroalkyl as defined above. For heteroalkylene groups, heteroatoms can also occupy either or both of the chain termini.

As used herein, the terms “aryl having 6 to 10 ring atoms” refer to a polyunsaturated, aromatic hydrocarbyl group having a single ring or multiple aromatic rings fused together, containing 6 to 10 ring atoms, wherein at least one ring is aromatic. The aromatic ring may optionally include one to two additional rings (cycloalkyl, heterocyclyl or heteroaryl as defined herein) fused thereto.

Suitable aryl groups include phenyl, naphtyl and phenyl ring fused to a heterocyclyl, like benzopyranyl, benzodioxolyl, benzodioxanyl and the like.

As used herein, the terms “heteroaryl having 5 to 10 ring atoms” refer to a polyunsaturated, aromatic ring system having a single ring or multiple aromatic rings fused together or linked covalently, containing 5 to 10 atoms, wherein at least one ring is aromatic and at least one ring atom is a heteroatom selected from N, O and S. The nitrogen and sulfur heteroatoms may optionally be oxidized and the nitrogen heteroatoms may optionally be quaternized. Such rings may be fused to an aryl, cycloalkyl or heterocyclyl ring. Non-limiting examples of such heteroaryl, include: furanyl, thiophenyl, pyrrolyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, indolyl, isoindolyl, benzofuranyl, isobenzofuranyl, benzothiophenyl, isobenzothiophenyl, indazolyl, benzimidazolyl, benzoxazolyl, purinyl, benzothiadiazolyl, quinolinyl, isoquinolinyl, cinnolinyl, quinazolinyl and quinoxalinyl.

As used herein, the terms “heterocyclyl having 5 to 10 ring atoms” refer to a saturated or unsaturated cyclic group having 5 to 10 ring atoms, wherein at least one ring atom is a heteroatom selected from N, O and S. The nitrogen and sulfur heteroatoms may optionally be oxidized and the nitrogen heteroatoms may optionally be quaternized. The heterocycle can include fused or bridged rings as well as spirocyclic rings. Examples of heterocycle include, but are not limited to, tetrahydropyridyl, piperidinyl, morpholinyl, tetrahydrofuranyl, tetrahydrothienyl, piperazinyl, 1-azepanyl, imidazolinyl, 1,4-dioxanyl and the like.

As used herein, the terms “organyl group” refer to any organic substituent group, regardless of functional type, having one free valence at a carbon atom.

As used herein, the terms “optionally substituted” can refer to groups that can be substituted with one or more of the substituents independently selected from: —X, —R′, —O—, —OR′, ═O, —SR′, —S—, —NR′₂, —NR′₃, ═NR′, —CX₃, —CN, —OCN, —SCN, —NCS, —NO, —NO₂, ═N₂, —NRC(═O)R′, —C(═O)R′, —C(═O)NR′₂, —SO₃, —SO₃H, —S(═O)₂R′, —OS(═O)₂OR′, —S(═O)₂NR′, —S(═O)R′, —C(═O)R′, —C(═S)R′, —CO₂R′, —CO₂, —C(═S)OR′, C(═O)SR′, C(═S)SR′, C(═O)NR′₂, C(═S)NR′₂, and C(═NR′)NR′₂, where each X is independently a halogen: —F, —C, —Br, or —I; and each R′ is independently —H, —C₁-C₂₀ alkyl, —C₆-C₁₀ aryl, or —C₃-C₁₀ heterocycle.

In the present disclosure, the dotted bond represents the attachment point of a moiety to the rest of the molecule.

Various embodiments of the disclosure are described herein. It will be recognized that features specified in each embodiment may be combined with other specified features to provide further embodiments.

The present disclosure encompasses the compounds of the present disclosure, their tautomers, enantiomers, diastereomers, racemates or mixtures thereof, and their hydrates, esters, solvates or pharmaceutically acceptable salts.

The terms “pharmaceutically acceptable salts” refers to salts that retain the biological effectiveness and properties of the compounds of this disclosure and, which typically are not biologically or otherwise undesirable. In many cases, the compounds of the disclosure are capable of forming acid and/or base salts by virtue of the presence of amino and/or carboxyl groups or groups similar thereto. Pharmaceutically acceptable acid addition salts can be formed with organic acids and/or inorganic acids. Pharmaceutically acceptable base addition salts can be formed with organic bases and/or inorganic bases.

In many cases, the compounds of the disclosure are capable of forming esters by virtue of the presence of carboxyl groups. Esters include C₁-C₆ alkyl esters.

Any formula given herein is also intended to represent unlabeled as well as isotopically forms of the compounds, like deuterium labeled compounds or ¹⁴C-labeled compounds.

### Compound of Formula (I)

The present disclosure first relates to a compound of formula (I):

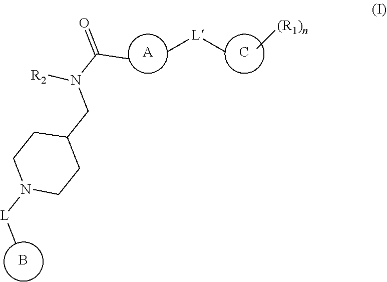

Wherein
Each R₁ is independently selected from halogen;
n is an integer between 0 and 5, preferably between 1 and 5;
Ring A is an heteroaryl having 5 ring atoms;
L′ is a bond or a divalent linker selected from the group consisting of C₁-C₆ alkylene, —O—, and C₁-C₆ heteroalkylene;
Ring C is a ring selected from the group consisting of C₃-C₁₂ cycloalkyl, heterocyclyl having 5 to 10 ring atoms, aryl having 6 to 10 ring atoms, and heteroaryl having 5 to 10 ring atoms;
R₂ is selected from H, C₁-C₆ alkyl, C₃-C₆ cycloalkyl, heterocyclyl having 5 to 10 ring atoms, aryl having 6 to 10 ring atoms, and heteroaryl having 5 to 10 ring atoms; said alkyl, cycloalkyl, heterocyclyl, aryl and heteroaryl being optionally substituted with one or more substituents, said substituents being preferably independently selected from oxo, halogen, —OH, —NO₂, —CN, and C₁-C₆ alkyl, preferably R2 is H;
L is a bond or a divalent linker selected from the group consisting of —(CR₃R₄)ₚ— p being an integer between 1 and 12, C₁-C₁₂ alkylene C₁-C₁₂ heteroalkylene, C₃-C₆ cycloalkylene, C₂-C₁₂ alkenylene, C₂-C₁₂ alkynylene, —CO—, —O—, —NR′—, —S—, —SO—, and —SO₂—, said heteroalkylene, alkenylene, and alkynylene being optionally interspersed with one or more groups selected from —(C₃-C₆ cycloalkyl)-, and said alkylene, heteroalkylene, cycloalkylene, alkenylene, and alkynylene being optionally substituted with one or more substituents, said substituents being preferably independently selected from oxo, halogen, —OH, —NO₂, —CN, C₁-C₆ alkyl, C₃-C₆ cycloalkyl, heterocyclyl having 5 to 10 ring atoms, aryl having 6 to 10 ring atoms, heteroaryl having 5 to 10 ring atoms, C₁-C₆ alkoxy, C₁-C₆ haloalkyl, C₁-C₆ haloalkoxy, —(CO)—R′, —O—(CO)—R′, —(CO)—O—R′, —(CO)—NR″R″′, —NR″—(CO)—R′, and —NR″R″;
Ring B is a ring selected from the group consisting of C₃-C₁₂ cycloalkyl, heterocyclyl having 5 to 10 ring atoms, aryl having 6 to 10 ring atoms, and heteroaryl having 5 to 10 ring atoms, said cycloalkyl, heterocyclyl, aryl and heteroaryl being optionally substituted with one or more substituents, said substituents being preferably independently selected from oxo, halogen, —OH, —NO₂, —CN, optionally substituted C₁-C₆ alkyl, optionally substituted C₃-C₆ cycloalkyl, optionally substituted heterocyclyl having 5 to 10 ring atoms, optionally substituted aryl having 6 to 10 ring atoms, optionally substituted heteroaryl having 5 to 10 ring atoms, optionally substituted C₁-C₆ alkoxy, C₁-C₆ haloalkyl, C₁-C₆ haloalkoxy, —(CO)—R′, —O—(CO)—R′, —(CO)—O—R′, —(CO)—NR″R″′, —NR″—(CO)—R′, —NR″R″′, —S(O)₂—R′, and S(O)₂—NR″R″′;
R₃ and R₄ are independently selected from H, C₁-C₆ alkyl, C₃-C₆ cycloalkyl, heterocyclyl having 5 to ring atoms, aryl having 6 to 10 ring atoms, heteroaryl having 5 to 10 ring atoms; said alkyl, cycloalkyl, heterocyclyl, aryl and heteroaryl being optionally substituted with one or more substituents, said substituents being preferably independently selected from oxo, halogen, —OH, —NO₂, —CN, and C₁-C₆ alkyl;
Or R₃ and R₄, together with the C atom to which they are bonded, form a C₃-C₁₂ cycloalkyl or a heterocyclyl having 5 to 10 ring atoms;
R′ is selected from H, C₁-C₆ alkyl, C₃-C₆ cycloalkyl, heterocyclyl having 5 to 10 ring atoms, aryl having 6 to 10 ring atoms, and heteroaryl having 5 to 10 ring atoms;
R″ and R″′ are independently selected from H, C₁-C₆ alkyl, C₃-C₆ cycloalkyl, heterocyclyl having 5 to ring atoms, aryl having 6 to 10 ring atoms, heteroaryl having 5 to 10 ring atoms; said alkyl, cycloalkyl, heterocyclyl, aryl and heteroaryl being optionally substituted with one or more substituents, said substituents being preferably independently selected from oxo, halogen, —OH, —NO₂, —CN, and C₁-C₆ alkyl;
Or R″ and R″′, together with the N atom to which they are linked, form a heterocycle having 5 to 10 ring atoms, or a heteroaryl having 5 to 10 ring atoms, said heterocyclyl, and heteroaryl being optionally substituted with one or more substituents, said substituents being preferably independently selected from oxo, halogen, —OH, —NO₂, —CN, and C₁-C₆ alkyl;
and pharmaceutically acceptable salts, solvates and esters thereof.

According to a preferred embodiment, the compound of formula (I) is a modulator of ACKR3.

As used herein, the term “ACKR3 modulator” refers to a compound which directly binds to ACKR3, and which competitively inhibits the binding of reference ACKR3 binding compound. For example, an ACKR3 modulator of the present disclosure is a compound which directly binds to ACKR3 and is capable of displacing the binding of a fluorescently labelled chemokine CXCL12, as measured in vitro, typically as measured with the displacement assay using a chemokine CXCL12 fluorescently labelled with the dye AF647® (Alexa Fluor 647). The dye Alexa Fluor 647 is a far-red-fluorescent cyanine dye. The fluorescently labelled chemokine CXCL12 can be h-SDF-1α (AF647). More details of such displacement assay are described in the Examples. In a more specific embodiment, said ACKR3 modulator of the present disclosure has preferably a pK, greater than or equal to 5.0, typically comprised between 5.0 and 9.0, and for example comprised between 5.0 and 8.4, as measured by the displacement assay with a fluorescently labelled chemokine CXCL12. More preferably, said ACKR3 modulator of the present disclosure has a pK, greater than or equal to 5.0, or at least 6.0, or at least 7.0.

In a more specific embodiment, said ACKR3 modulator of the present disclosure has preferably a pK, greater than or equal to 5.0, typically comprised between 5.0 and 9.0, and for example comprised between 6.0 and 8.5, as measured by the displacement assay with labelled ACKR3 binding reference compound: a fluorescently labelled chemokine CXCL12. More preferably, said ACKR3 modulator of the present disclosure has a pK, greater than or equal to 5.0, or at least 6.0, or at least 7.0.

The compounds of the disclosure are modulators of ACKR3. They have enhanced pharmacokinetic properties, and good metabolic stability and affinity. Moreover, the compounds of the disclosure have a low structural complexity as they can have no chiral center, which is an advantage compared to known modulators of ACKR3 that contain a chiral center. Indeed, the lack of chiral center leads to an effective synthesis, as it does not require extra step or enantiopure reagents, which are often expensive, to produce enantiopure compounds. The synthesis of the compounds of the disclosure is thus easy and cost effective.

According to an embodiment, each R₁ is F. According to an embodiment, n is an integer between 1 and 5, or between 2 and 5. According to an embodiment, n is 2.

According to an embodiment, ring A is a heteroaryl having 5 ring atoms, including at least 2 heteroatoms independently selected from N, O and S. Ring A can be selected from the group consisting of imidazole, pyrazole, triazole, tetrazole, oxazole, isoxazole, isothiazole, thiazole, thiadiazole and oxadiazole.

According to an embodiment, ring A is selected from the group consisting o

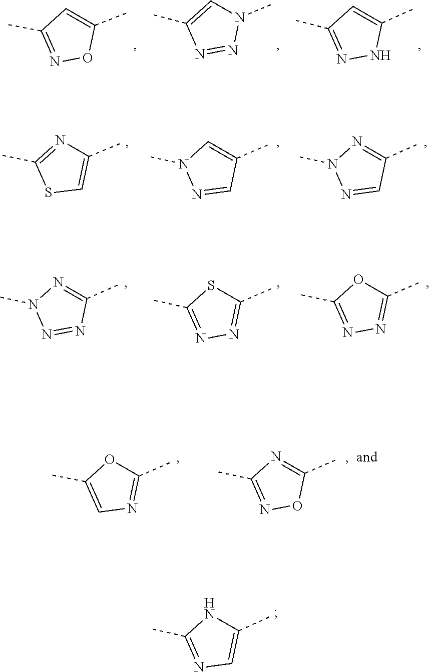

preferably ring A is

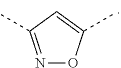

According to an embodiment, L is a divalent linker selected from C₃-C₁₂ alkylene, C₃-C₆ cycloalkylene, C₃-C₁₂ alkenylene, and C₃-C₁₂ alkynylene.

According to an embodiment, L is a bond or a divalent linker selected from C₁-C₁₂ alkylene, C₂-C₁₂ alkenylene, —CO—, and —SO₂—. According to an embodiment, L is a bond or a divalent linker selected from C₃-C₁₂ alkylene, C₂-C₁₂ alkenylene, and —CO—.

According to an embodiment, L′ is a bond or a divalent linker selected from the group consisting of C₁-C₆ alkylene, preferably L′ is a bond.

According to an embodiment, ring B is an aryl having 6 to 10 ring atoms, said aryl being optionally substituted with one or more substituents independently selected from halogen, —OH, C₁-C₆ alkyl, C₁-C₆ alkoxy, —(CO)—O—R′, —NR″—(CO)—R′, and —NR″R″′.

According to an embodiment, the compound of formula (I) is a compound of formula (II)

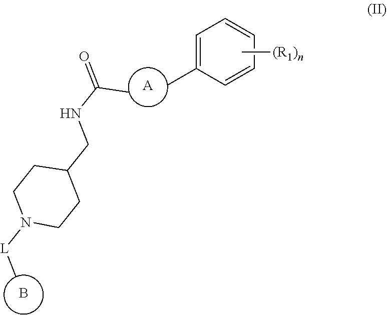

According to an embodiment, the compound of formula (I) is a compound of formula (II) wherein
Each R₁ is independently selected from halogen;
n is an integer between 2 and 5, preferably n is 2;
Ring A is an heteroaryl having 5 ring atoms;
L is a bond or a divalent linker selected from the group consisting of —(CR₃R₄)ₚ— p being an integer between 1 and 12, C₁-C₁₂ alkylene C₁-C₁₂ heteroalkylene, C₃-C₆ cycloalkylene, C₂-C₁₂ alkenylene, C₂-C₁₂ alkynylene, —CO—, —O—, —NR′—, —S—, —SO—, and —SO₂—, said heteroalkylene, alkenylene, and alkynylene being optionally interspersed with one or more groups selected from —(C₃-C₆ cycloalkyl)-, and said alkylene, heteroalkylene, cycloalkylene, alkenylene, and alkynylene being optionally substituted with one or more substituents, said substituents being preferably independently selected from oxo, halogen, —OH, —NO₂, —CN, C₁-C₆ alkyl, C₃-C₆ cycloalkyl, heterocyclyl having 5 to 10 ring atoms, aryl having 6 to 10 ring atoms, heteroaryl having 5 to 10 ring atoms, C₁-C₆ alkoxy, C₁-C₆ haloalkyl, C₁-C₆ haloalkoxy, —(CO)—R′, —O—(CO)—R′, —(CO)—O—R′, —(CO)—NR″R″′, —NR″—(CO)—R′, and —NR″R″;
Ring B is a ring selected from the group consisting of C₃-C₁₂ cycloalkyl, heterocyclyl having 5 to 10 ring atoms, aryl having 6 to 10 ring atoms, and heteroaryl having 5 to 10 ring atoms, said cycloalkyl, heterocyclyl, aryl and heteroaryl being optionally substituted with one or more substituents, said substituents being preferably independently selected from oxo, halogen, —OH, —NO₂, —CN, optionally substituted C₁-C₆ alkyl, optionally substituted C₃-C₆ cycloalkyl, optionally substituted heterocyclyl having 5 to 10 ring atoms, optionally substituted aryl having 6 to 10 ring atoms, optionally substituted heteroaryl having 5 to 10 ring atoms, optionally substituted C₁-C₆ alkoxy, C₁-C₆ haloalkyl, C₁-C₆ haloalkoxy, —(CO)—R′, —O—(CO)—R′, —(CO)—O—R′, —(CO)—NR″R″′, —NR″—(CO)—R′, —NR″R″′, —S(O)₂—R′, and S(O)₂—NR″R″′;
R₃ and R₄ are independently selected from H, C₁-C₆ alkyl, C₃-C₆ cycloalkyl, heterocyclyl having 5 to ring atoms, aryl having 6 to 10 ring atoms, heteroaryl having 5 to 10 ring atoms; said alkyl, cycloalkyl, heterocyclyl, aryl and heteroaryl being optionally substituted with one or more substituents, said substituent being preferably independently selected from oxo, halogen, —OH, —NO₂, —CN, and C₁-C₆ alkyl;
Or R₃ and R₄, together with the C atom to which they are bonded, form a C₃-C₁₂ cycloalkyl or a heterocyclyl having 5 to 10 ring atoms;
R′ is selected from H, C₁-C₆ alkyl, C₃-C₆ cycloalkyl, heterocyclyl having 5 to 10 ring atoms, aryl having 6 to 10 ring atoms, and heteroaryl having 5 to 10 ring atoms;
R″ and R″′ are independently selected from H, C₁-C₆ alkyl, C₃-C₆ cycloalkyl, heterocyclyl having 5 to ring atoms, aryl having 6 to 10 ring atoms, heteroaryl having 5 to 10 ring atoms; said alkyl, cycloalkyl, heterocyclyl, aryl and heteroaryl being optionally substituted with one or more substituents, said substituent being preferably independently selected from oxo, halogen, —OH, —NO₂, —CN, and C₁-C₆ alkyl;
Or R″ and R″′, together with the N atom to which they are linked, form a heterocycle having 5 to 10 ring atoms, or a heteroaryl having 5 to 10 ring atoms, said heterocyclyl, and heteroaryl being optionally substituted with one or more substituents, said substituent being preferably independently selected from oxo, halogen, —OH, —NO₂, —CN, and C₁-C₆ alkyl.

According to an embodiment, the compound of formula (I) is a compound of formula (III)

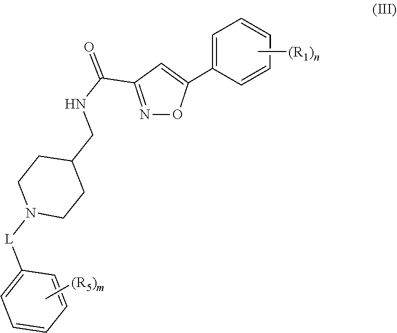

Wherein
Each R₁ is independently selected from halogen, provided that at least one R₁ is —F;
n is an integer between 1 and 5,
m is an integer from 0 to 5; and
each R₅ is selected from halogen, —OH, —NO₂, —CN, optionally substituted C₁-C₆ alkyl, optionally substituted C₃-C₆ cycloalkyl, optionally substituted heterocyclyl having 5 to 10 ring atoms, optionally substituted aryl having 6 to 10 ring atoms, optionally substituted heteroaryl having 5 to 10 ring atoms, optionally substituted C₁-C₆ alkoxy, C₁-C₆ haloalkyl, C₁-C₆ haloalkoxy, —(CO)—R′, —O—(CO)—R′, —(CO)—O—R′, —(CO)—NR″R″′, —NR″—(CO)—R′, —NR″R″′, —S(O)₂—R′, and S(O)₂—NR″R″′.

According to an embodiment, the compound of formula (I) is a compound of formula (IV)

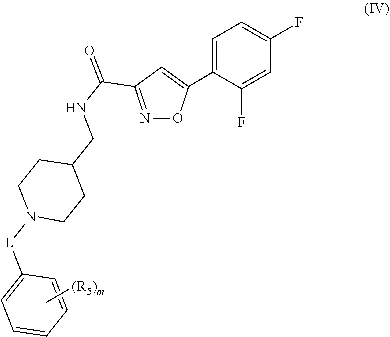

Wherein
m is an integer from 0 to 3; and
each R₅ is selected from halogen, —OH, —NO₂, —CN, optionally substituted C₁-C₆ alkyl, optionally substituted C₃-C₆ cycloalkyl, optionally substituted heterocyclyl having 5 to 10 ring atoms, optionally substituted aryl having 6 to 10 ring atoms, optionally substituted heteroaryl having 5 to 10 ring atoms, optionally substituted C₁-C₆ alkoxy, C₁-C₆ haloalkyl, C₁-C₆ haloalkoxy, —(CO)—R′, —O—(CO)—R′, —(CO)—O—R′, —(CO)—NR″R″′, —NR″—(CO)—R′, —NR″R″′, —S(O)₂—R′, and S(O)₂—NR″R″′.

According to an embodiment, the compound of formula (I) is a compound of formula (V)

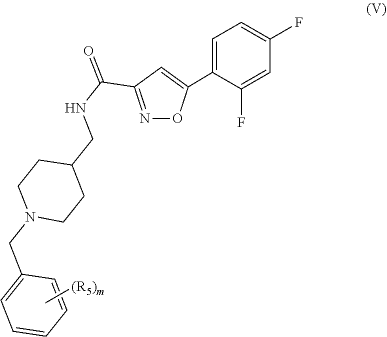

Wherein
m is an integer from 0 to 3; and
each R₅ is selected from oxo, halogen, —OH, —NO₂, —CN, C₁-C₆ alkyl, C₃-C₆ cycloalkyl, heterocyclyl having 5 to 10 ring atoms, aryl having 6 to 10 ring atoms, heteroaryl having 5 to 10 ring atoms, C₁-C₆ alkoxy, C₁-C₆ haloalkyl, C₁-C₆ haloalkoxy, —(CO)—R′, —O—(CO)—R′, —(CO)—O—R′, —(CO)—NR″R″′, —NR″—(CO)—R′, —NR″R″′, —S(O)₂—R′, and S(O)₂—NR″R″′, preferably from halogen, —OH, —NO₂, —CN, C₁-C₆ alkyl, C₃-C₆ cycloalkyl, heterocyclyl having 5 to 10 ring atoms, aryl having 6 to 10 ring atoms, heteroaryl having 5 to 10 ring atoms, C₁-C₆ alkoxy, C₁-C₆ haloalkyl, C₁-C₆ haloalkoxy, —(CO)—R′, —O—(CO)—R′, —(CO)—O—R′, —(CO)—NR″R″′, —NR″—(CO)—R′, —NR″R″′, —S(O)₂—R′, and S(O)₂—NR″R″′.

According to an embodiment, the compound of formula (I) is a compound of formula (VI)

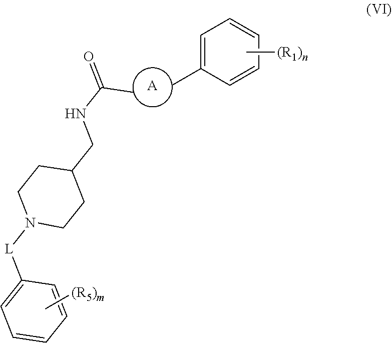

wherein
Each R₁ is independently selected from halogen;
n is an integer between 2 and 5, preferably n is 2;
Ring A is an heteroaryl having 5 ring atoms;
L is a bond or a divalent linker selected from the group consisting of —(CR₃R₄)ₚ— p being an integer between 1 and 12, C₁-C₁₂ alkylene C₁-C₁₂ heteroalkylene, C₃-C₆ cycloalkylene, C₂-C₁₂ alkenylene, C₂-C₁₂ alkynylene, —CO—, —O—, —NR′—, —S—, —SO—, and —SO₂—, said heteroalkylene, alkenylene, and alkynylene being optionally interspersed with one or more groups selected from —(C₃-C₆ cycloalkyl)-, and said alkylene, heteroalkylene, cycloalkylene, alkenylene, and alkynylene being optionally substituted with one or more substituents, said substituents being preferably independently selected from oxo, halogen, —OH, —NO₂, —CN, C₁-C₆ alkyl, C₃-C₆ cycloalkyl, heterocyclyl having 5 to 10 ring atoms, aryl having 6 to 10 ring atoms, heteroaryl having 5 to 10 ring atoms, C₁-C₆ alkoxy, C₁-C₆ haloalkyl, C₁-C₆ haloalkoxy, —(CO)—R′, —O—(CO)—R′, —(CO)—O—R′, —(CO)—NR″R″′, —NR″—(CO)—R′, and —NR″R″′;
m is an integer from 0 to 5; and
each R₅ is selected from halogen, —OH, —NO₂, —CN, optionally substituted C₁-C₆ alkyl, optionally substituted C₃-C₆ cycloalkyl, optionally substituted heterocyclyl having 5 to 10 ring atoms, optionally substituted aryl having 6 to 10 ring atoms, optionally substituted heteroaryl having 5 to 10 ring atoms, optionally substituted C₁-C₆ alkoxy, optionally substituted C₁-C₆ haloalkyl, C₁-C₆ haloalkoxy, —(CO)—R′, —O—(CO)—R′, —(CO)—O—R′, —(CO)—NR″R″′, —NR″—(CO)—R′, —NR″R″′, —S(O)₂—R′, and S(O)₂—NR″R″′.

According to an embodiment, the compound of formula (I) is a compound of formula (VII)

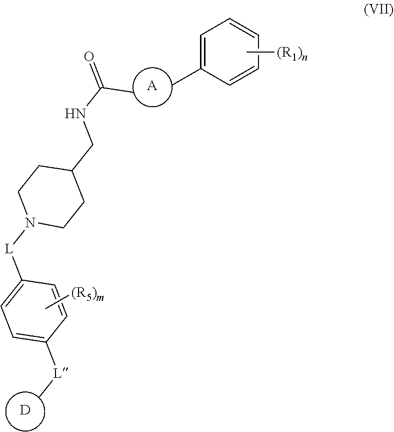

wherein
Each R₁ is independently selected from halogen;
n is an integer between 0 and 5, preferably between 2 and 5;
Ring A is an heteroaryl having 5 ring atoms;
L is a bond or a divalent linker selected from the group consisting of —(CR₃R₄)ₚ— p being an integer between 1 and 12, C₁-C₁₂ alkylene C₁-C₁₂ heteroalkylene, C₃-C₆ cycloalkylene, C₂-C₁₂ alkenylene, C₂-C₁₂ alkynylene, —CO—, —O—, —NR′—, —S—, —SO—, and —SO₂—, said heteroalkylene, alkenylene, and alkynylene being optionally interspersed with one or more groups selected from —(C₃-C₆ cycloalkyl)-, and said alkylene, heteroalkylene, cycloalkylene, alkenylene, and alkynylene being optionally substituted with one or more substituents, said substituents being preferably independently selected from oxo, halogen, —OH, —NO₂, —CN, C₁-C₆ alkyl, C₃-C₆ cycloalkyl, heterocyclyl having 5 to 10 ring atoms, aryl having 6 to 10 ring atoms, heteroaryl having 5 to 10 ring atoms, C₁-C₆ alkoxy, C₁-C₆ haloalkyl, C₁-C₆ haloalkoxy, —(CO)—R′, —O—(CO)—R′, —(CO)—O—R′, —(CO)—NR″R″′, —NR″—(CO)—R′, and —NR″R″′;
L″ is a bond or a divalent linker selected from the group consisting of —(CR₃R₄)ₚ— p being an integer between 1 and 12, C₁-C₁₂ alkylene C₁-C₁₂ heteroalkylene, C₃-C₆ cycloalkylene, C₂-C₁₂ alkenylene, C₂-C₁₂ alkynylene, —CO—, —O—, —NR′—, —S—, —SO—, and —SO₂—, said heteroalkylene, alkenylene, and alkynylene being optionally interspersed with one or more groups selected from —(C₃-C₆ cycloalkyl)-, and said alkylene, heteroalkylene, cycloalkylene, alkenylene, and alkynylene being optionally substituted with one or more substituents, said substituents being preferably independently selected from oxo, halogen, —OH, —NO₂, —CN, C₁-C₆ alkyl, C₃-C₆ cycloalkyl, heterocyclyl having 5 to 10 ring atoms, aryl having 6 to 10 ring atoms, heteroaryl having 5 to 10 ring atoms, C₁-C₆ alkoxy, C₁-C₆ haloalkyl, C₁-C₆ haloalkoxy, —(CO)—R′, —O—(CO)—R′, —(CO)—O—R′, —(CO)—NR″R″′, —NR″—(CO)—R′, and —NR″R″′;
m is an integer from 0 to 4; and
each R₅ is selected from halogen, —OH, —NO₂, —CN, optionally substituted C₁-C₆ alkyl, optionally substituted C₁-C₆ alkoxy, optionally substituted C₁-C₆ haloalkyl, C₁-C₆ haloalkoxy, —(CO)—R′, —O—(CO)—R′, —(CO)—O—R′, —(CO)—NR″R″′, —NR″—(CO)—R′, —NR″R″′, —S(O)₂—R′, and S(O)₂-NR″R″′;
Ring D is a ring selected from the group consisting of C₃-C₁₂ cycloalkyl, heterocyclyl having 5 to 10 ring atoms, aryl having 6 to 10 ring atoms, and heteroaryl having 5 to 10 ring atoms, said cycloalkyl, heterocyclyl, aryl and heteroaryl being optionally substituted with one or more substituents, said substituents being preferably independently selected from oxo, halogen, —OH, —NO₂, —CN, optionally substituted C₁-C₆ alkyl, optionally substituted C₃-C₆ cycloalkyl, optionally substituted heterocyclyl having 5 to 10 ring atoms, optionally substituted aryl having 6 to 10 ring atoms, optionally substituted heteroaryl having 5 to 10 ring atoms, optionally substituted C₁-C₆ alkoxy, C₁-C₆ haloalkyl, C₁-C₆ haloalkoxy, —(CO)—R′, —O—(CO)—R′, —(CO)—O—R′, —(CO)—NR″R″′, —NR″—(CO)—R′, —NR″R″′, —S(O)₂—R′, and S(O)₂—NR″R″′.

According to an embodiment, L″ is a bond, and Ring D is a ring selected from the group consisting of heterocyclyl having 5 to 10 ring atoms, and heteroaryl having 5 to 10 ring atoms.

According to an embodiment, the compound of formula (I) is selected from

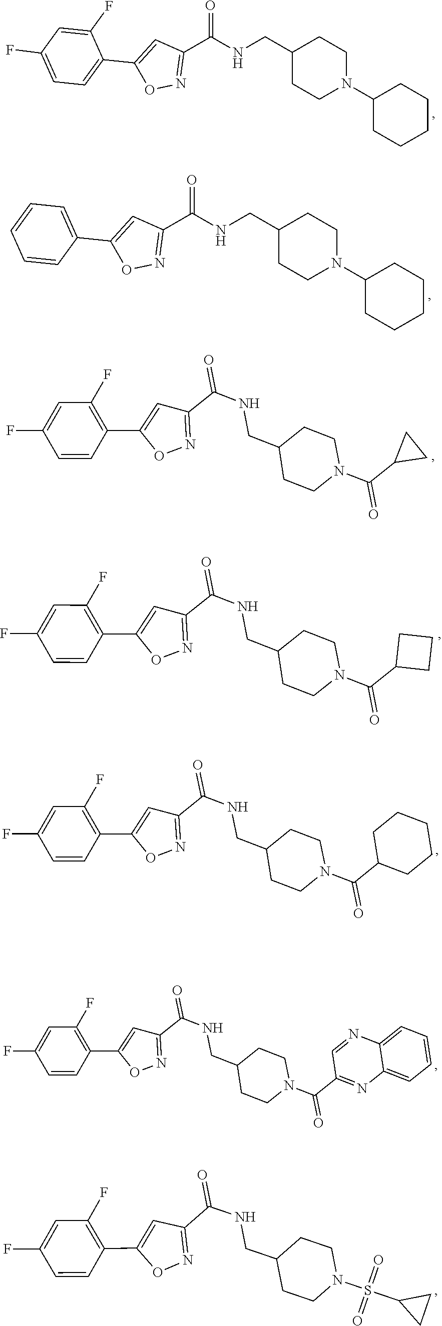

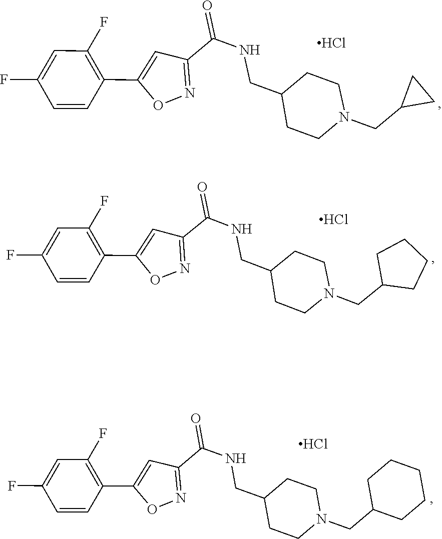 [SMILES: O=C(NCC1CCN(CC2CC2)CC1)c1cc(-c2ccc(F)cc2F)on1 ; Cl ; O=C(NCC1CCN(CC2CCCC2)CC1)c1cc(-c2ccc(F)cc2F)on1 ; Cl ; O=C(NCC1CCN(CC2CCCCC2)CC1)c1cc(-c2ccc(F)cc2F)on1 ; Cl]

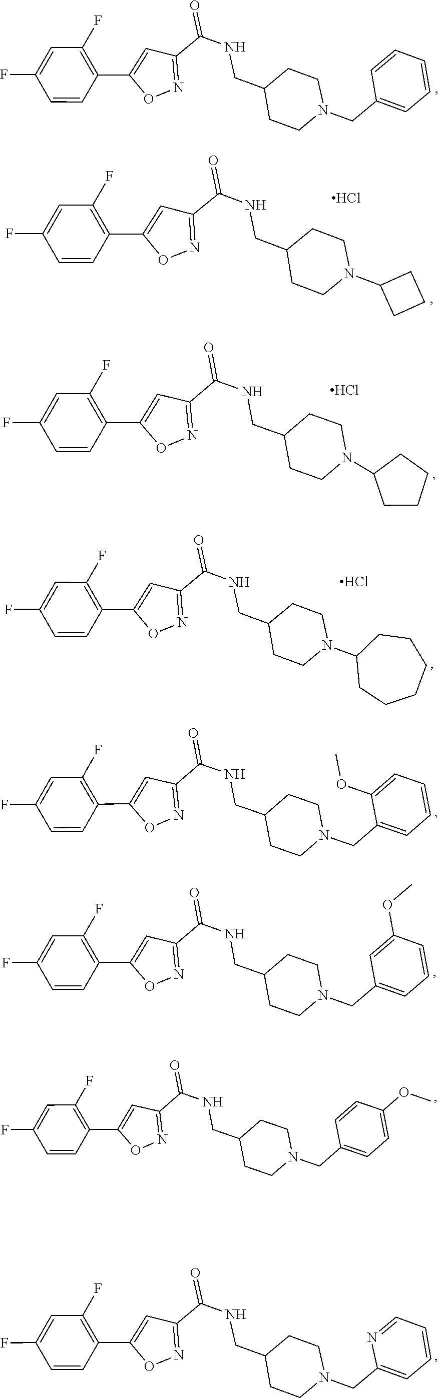

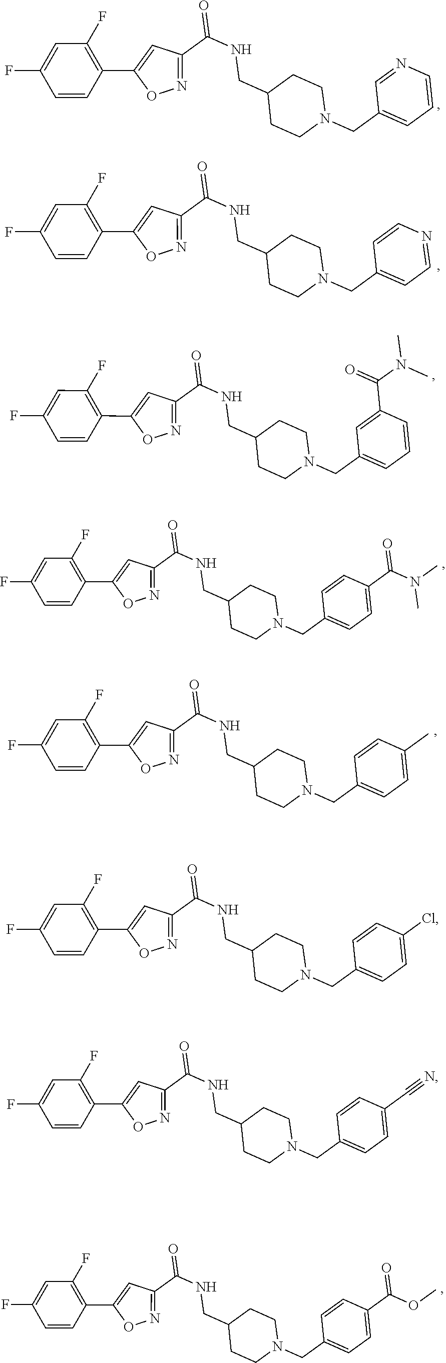

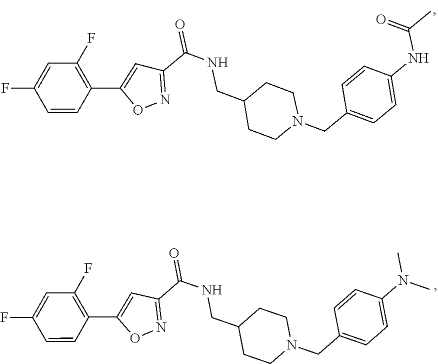 [SMILES: CC(=O)Nc1ccc(CN2CCC(CNC(=O)c3cc(-c4ccc(F)cc4F)on3)CC2)cc1 ; CN(C)c1ccc(CN2CCC(CNC(=O)c3cc(-c4ccc(F)cc4F)on3)CC2)cc1]

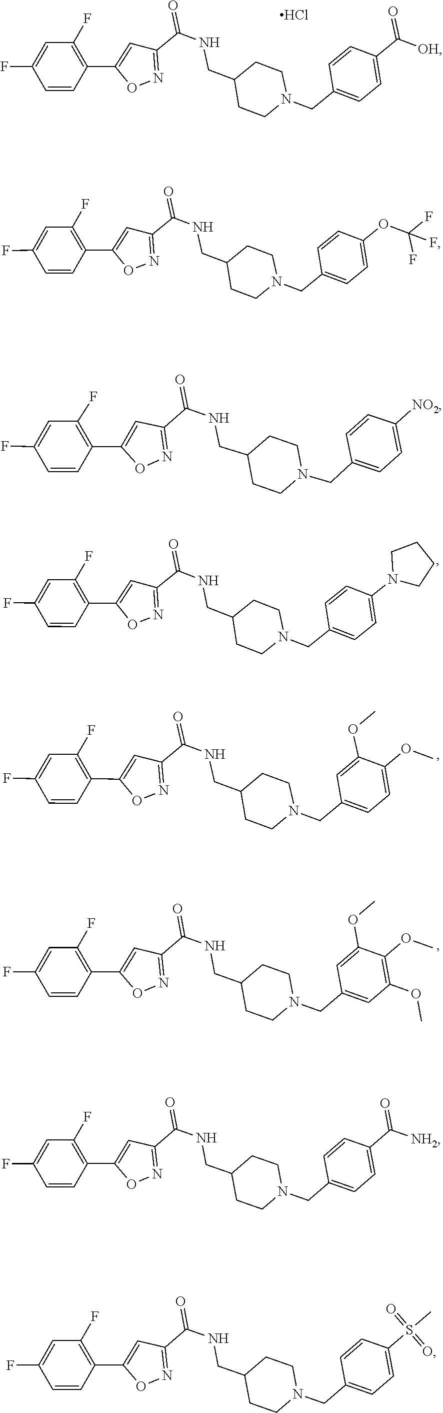

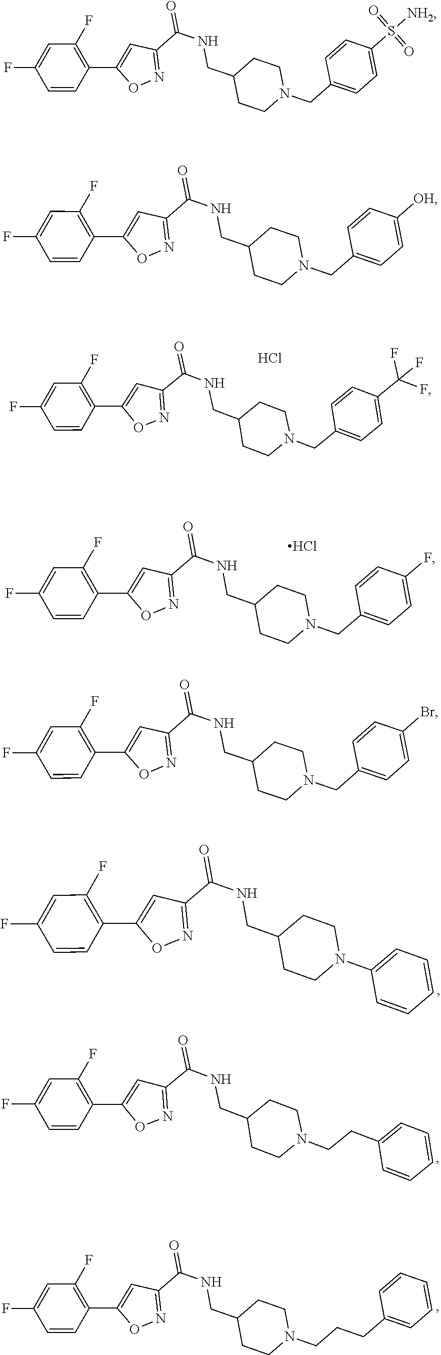

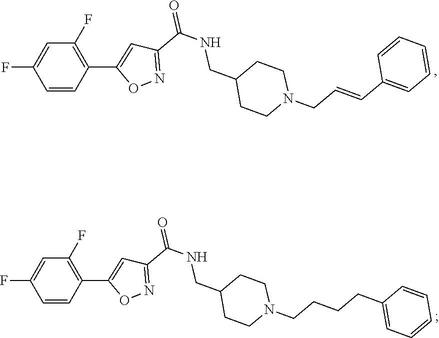 [SMILES: O=C(NCC1CCN(C/C=C/c2ccccc2)CC1)c1cc(-c2ccc(F)cc2F)on1 ; O=C(NCC1CCN(CCCCc2ccccc2)CC1)c1cc(-c2ccc(F)cc2F)on1]

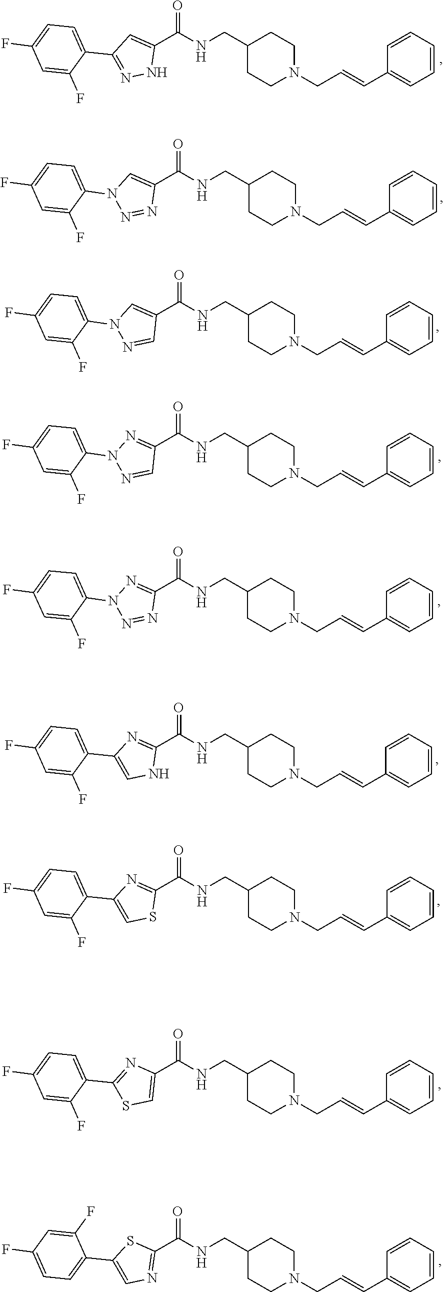

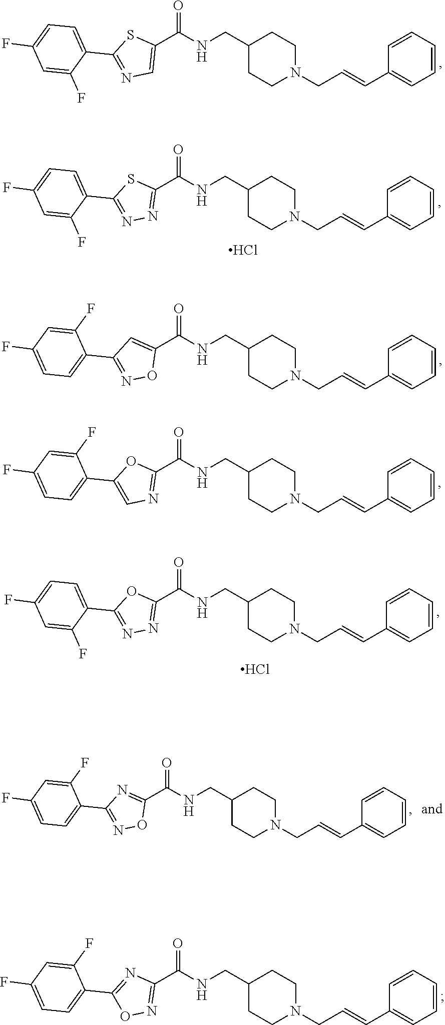

preferably, the compound is selected from

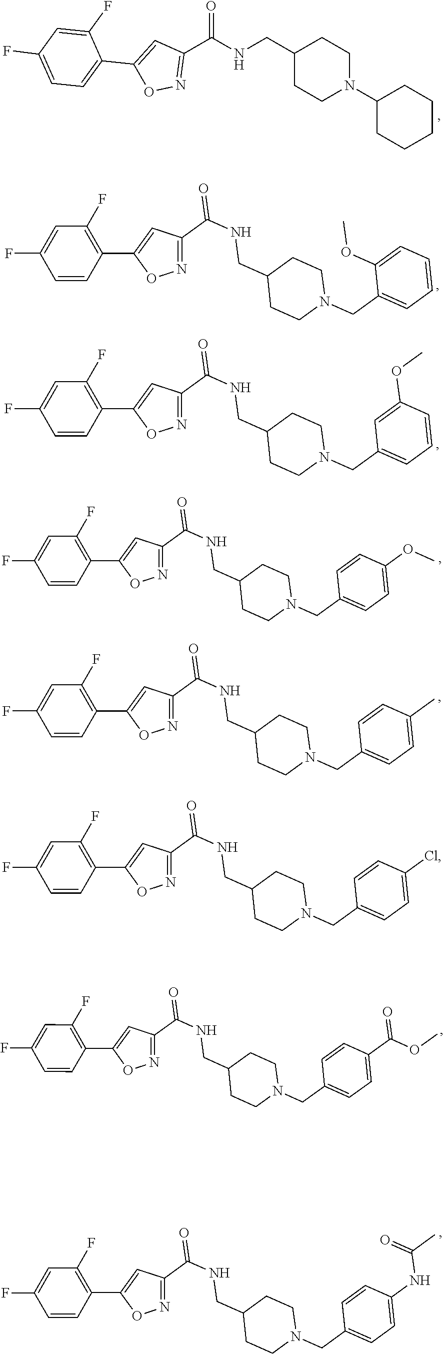

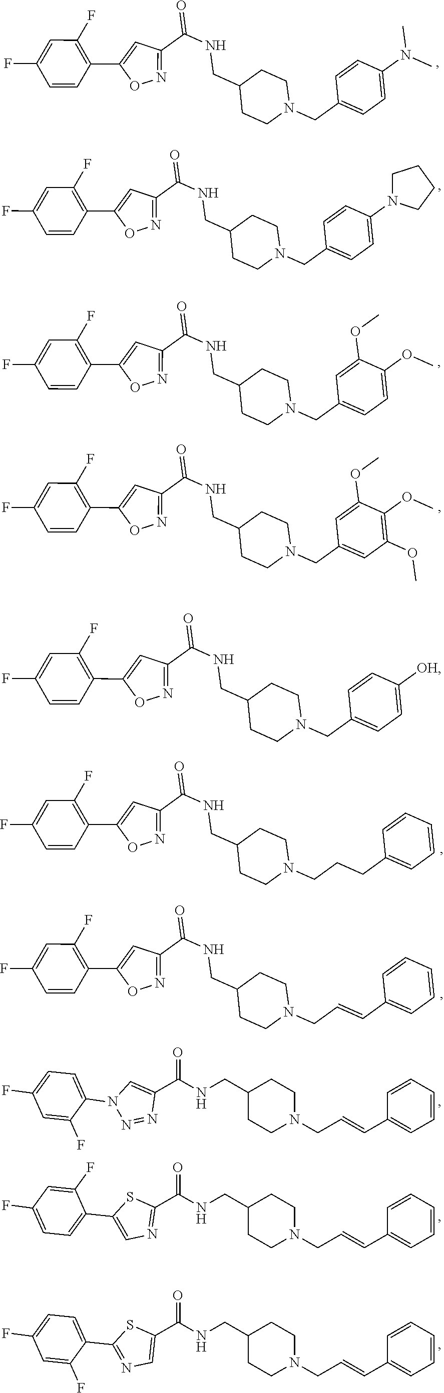

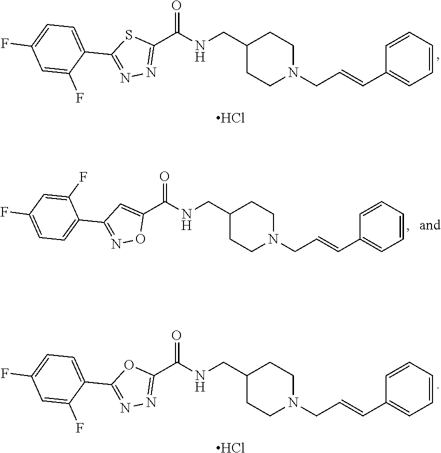 [SMILES: O=C(NCC1CCN(C/C=C/c2ccccc2)CC1)c1nnc(-c2ccc(F)cc2F)s1 ; Cl ; O=C(NCC1CCN(C/C=C/c2ccccc2)CC1)c1cc(-c2ccc(F)cc2F)no1 ; O=C(NCC1CCN(C/C=C/c2ccccc2)CC1)c1nnc(-c2ccc(F)cc2F)o1 ; Cl]

### Pharmaceutical Composition

The disclosure also relates to a pharmaceutical composition comprising a compound of the disclosure and at least one pharmaceutically acceptable carrier.

The form of the pharmaceutical compositions, the route of administration, the dosage and the regimen naturally depend upon the condition to be treated, the severity of the illness, the age, weight, and sex of the patient, etc.

The pharmaceutical compositions of the disclosure can be formulated for a topical, oral, intranasal, intraocular, intravenous, intramuscular or subcutaneous administration and the like.

The pharmaceutical compositions can take the form of tablets, pills, capsules, semisolids, powders, sustained release formulations, solutions, suspensions, emulsions, syrups, elixirs, aerosols, or any other appropriate compositions; and comprise at least one compound according this disclosure.

Because of their ease in administration, tablets and capsules represent the most advantageous oral dosage unit form, in which case solid pharmaceutical carriers are obviously employed. If desired, tablets may be sugar coated or enteric coated by standard techniques. The tablets or pills can be coated to provide a dosage form affording the advantage of prolonged action. For example, the tablet or pills can comprise an inner dosage and an outer dosage component, the latter being in the form of an envelope over the former. The two components can be separated by an enteric layer, which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The compound of the disclosure and the further agent may also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

According to an embodiment, the pharmaceutical compositions contain vehicles which are pharmaceutically acceptable for oral formulation.

According to an embodiment, the pharmaceutical compositions contain vehicles which are pharmaceutically acceptable for a formulation capable of being injected or infused. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions or solutions for infusion.

The pharmaceutical forms suitable for injection or infusion include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, mixtures thereof and in oils. Under ordinary conditions of storage and use, these preparations contain a preservative to prevent the growth of microorganisms.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions and solutions for infusion are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions or solutions for infusion, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution may be suitably buffered and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15^{th} Edition, pages 1035-1038 and 1570-1580). Some variation in dosage will necessarily occur depending on the condition of the subject being treated. The person responsible for administration will, in any event, determine the appropriate dose for the individual subject.

For aerosol administration, the compound of the disclosure and the further agent are preferably supplied in finely divided from along with a surfactant and propellant. The surfactant must, of course, be nontoxic, and preferably soluble in the propellant. Representative of such agents are the esters or partial esters of fatty acids containing from 6 to 22 carbon atoms, such as caproic, octanoic lauric, palmitic, stearic, linoleic, linolenic, olesteric and oleic acids with an aliphatic polyhydric alcohol or its cyclic anhydride. Mixed esters, such as mixed or natural glycerides may be employed. A carrier can also be included, as desired, as with, e.g., lecithin for intranasal delivery. An example includes a solution in which each milliliter included 7.5 mg NaCl, 1.7 mg citric acid monohydrate, 3 mg disodium phosphate dihydrate and 0.2 mg benzalkonium chloride solution (50%) (Gozes et al., J Mol Neurosci. 19(1-2):167-70 (2002)).

Suitable compositions for topical application include aqueous solutions, suspensions, ointments, creams, gels or sprayable formulations, e.g. aerosol administration.

The doses used for the administration can be adapted as a function of various parameters, and in particular as a function of the mode of administration used, of the relevant pathology, or alternatively of the desired duration of treatment. It will be appreciated that appropriate dosages of the compounds, and compositions comprising the compounds, can vary from patient to patient. Determining the optimal dosage will generally involve the balancing of the level of therapeutic benefit against any risk or deleterious side effects of the treatments described herein. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects. For example, the dose used for the administration can be of about 1-1000 mg of the compound of the disclosure for a subject of about 50-70 kg.

### Method of Use

The compounds of the disclosure exhibit valuable pharmaceutical properties as indicated in the in vitro tests and studies provided in the examples and are therefore indicated for therapy. In particular, the compounds of the disclosure are useful in the prevention or treatment of disorders relating to the ACKR3 receptor.

The disclosure also relates to a compound of the disclosure for use as a medicament.

The disclosure also relates to a compound of the disclosure for use in the treatment of cancer, autoimmune disorders, inflammatory diseases, transplant rejection, fibrosis, or pain.

The disclosure also relates to a compound of the disclosure for use in the treatment of disorders relating to the ACKR3 receptor.

In addition, further diseases or disorders relating to the ACKR3 receptor or its ligands are diseases involving ACKR3 and/or CXCL12 and/or CXCL11 mediated metastasis, chemotaxis, cell adhesion, trans-endothelial migration, cell proliferation and/or survival.

In addition, further particular diseases or disorders relating to the ACKR3 receptor or its ligands are proliferative diabetic retinopathy; West Nile virus encephalitis; pulmonary vascular diseases, acute renal failure, ischemia including cerebral ischemia, acute coronary syndrome, injured central nervous system, hypertension, pulmonary hypertension, Shiga-toxin-associated heomolytic uremic syndrome, preeclampsia, vascular injury, HIV/AIDS, angiogenesis, and brain and neuronal dysfunctions (such as inflammatory components of Alzheimer's disease), stress-related disorders (such as anxiety, depression, and posttraumatic stress disorder), and diseases involving opioid receptors. In a sub-embodiment, such a further particular disease or disorder relating to the ACKR3 receptor or its ligands is especially pulmonary hypertension.

As used herein, the term “cancer” has its general meaning in the art and includes an abnormal state or condition characterized by rapidly proliferating cell growth. The term is meant to include all types of cancerous growths or oncogenic processes, metastatic tissues or malignantly transformed cells, tissues or organs, irrespective of histopathologic type or stage of invasiveness.

The term cancer includes malignancies of the various organ systems, such as affecting skin, lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the oesophagus.

Examples of cancer include, but are not limited, to hematological malignancies such as B-cell lymphoid neoplasm, T-cell lymphoid neoplasm, non-hodgkin lymphoma (NHL), B-NHL, T-NHL, chronic lymphocytic leukemia (CLL), small lymphocytic lymphoma (SLL), mantle cell lymphoma (MCL), NK-cell lymphoid neoplasm, and myeloid cell lineage neoplasm. Examples of non-hematological cancers include, but are not limited to, skin cancer, colon cancer, breast cancer, lung cancer, brain cancer, prostate cancer, head and neck cancer, pancreatic cancer, bladder cancer, colorectal cancer, bone cancer, cervical cancer, liver cancer, oral cancer, oesophageal cancer, thyroid cancer, kidney cancer, stomach cancer and testicular cancer.

Examples of autoimmune disorders include (inflammatory) demyelinating diseases; multiple sclerosis (MS); Guillain Barre syndrome; rheumatoid arthritis (RA); inflammatory bowel diseases (IBD, especially comprising Crohn's disease and ulcerative colitis); systemic lupus erythematosus (SLE); lupus nephritis; interstitial cystitis; celiac disease; autoimmune encephalomyelitis; osteoarthritis; and type I diabetes. According to an embodiment, the autoimmune disorder is a demyelinating disease or multiple sclerosis.

Examples of inflammatory diseases include chronic rhinosinusitis, asthma, chronic obstructive pulmonary disorder, atherosclerosis, myocarditis, and sarcoidosis.

Examples of transplant rejection include renal allograft rejection, cardiac allograft rejection, and graft-versus-host diseases brought about by hematopoietic stem cell transplantation.

Examples of fibrosis include liver fibrosis, liver cirrhosis, lung fibrosis, especially idiopathic pulmonary fibrosis.

The disclosure relates to a method for treating cancer, autoimmune disorders, inflammatory diseases, transplant rejection, fibrosis, or pain, said method comprising administering to a subject in need thereof, preferably a human, a therapeutically efficient amount of
(i) a compound of the disclosure, or
(ii) a pharmaceutical composition as described herein.

The disclosure relates to a method for treating disorders relating to the ACKR3 receptor said method comprising administering to a subject in need thereof, preferably a human, a therapeutically efficient amount of
(i) a compound of the disclosure, or
(ii) a pharmaceutical composition as described herein.

As used herein, the term “treating” includes reversing, alleviating, inhibiting the progression of, preventing or reducing the likelihood of the disease, disorder, or condition to which such term applies, or one or more symptoms or manifestations of such disease, disorder or condition.

Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

As used herein, the terms “therapeutically efficient amount” of a compound refer to an amount of the compound that will elicit the biological or medical response of a subject, for example, ameliorate the symptoms, alleviate conditions, slow or delay disease progression, or prevent a disease.

The disclosure also relates to the use of a compound of the disclosure, for the manufacture of a medicament for the treatment of cancer, autoimmune disorders, inflammatory diseases, transplant rejection, fibrosis, or pain.

The disclosure also relates to the use of a compound of the disclosure, for the manufacture of a medicament for the treatment of disorders relating to the ACKR3 receptor.

### EXAMPLES

### Experimentals

### Pharmacology

### Binding Studies—Membrane Production

Membranes of HEK293T cells that transiently express the NanoLuc-hACKR3 were produced. 2 million HEK293T cells were seeded per 10 cm² dish and were transfected the next day using a DNA/PEI-mix containing 0.25 μg plasmid DNA encoding for the ACKR3, 4.75 μg pcDEF3 plasmid DNA and 30 μg linear PEI. Two days after transfection, cells were collected in PBS and pelleted by centrifugation. Cell pellets were resuspended in ice-cold membrane buffer (15 mM Tris, 0.3 mM EDTA, 2 mM MgCl2, pH 7.4 at 4° C.) and were dounce-homogenized on ice by plunging the pestle times with 1500 rpm. Cell homogenates were subsequently subjected to two freeze-thaw cycles using liquid nitrogen. Next, the cell homogenates were centrifuged at 40,000 g and pellets were resuspended in Tris-sucrose buffer (20 mM Tris, 250 mM Sucrose, pH 7.4 at 4° C.). Finally, the membrane samples were homogenized using a 23-gauge needle, snap-frozen with liquid nitrogen and stored until further experimentation at −80° C.

### Binding Studies—NanoBRET Binding

Binding reactions were measured in triplicate on white low-volume 384-well plates and started by combining 0.3 nM fluorescently labelled CXCL12 (CXCL12 labelled with a AF647® dye in a molar ratio of dye:peptide of 1:1, purchased from ALMAC, h-SDF-1α (AF647®), cat. No. CAF-11, Sequence:

KPVSLSYRCPCRFFESHVARANVKHLKILNTPNCALQIVARLKNNNRQVCIDPKLKWIQEYLEK(Ale xa647®)ALN), increasing concentrations of test compound (10⁻⁵ M-10⁻¹¹ M) with 35 ng membranes (protein content) per well of HEK293T cells expressing the NanoLuc-ACKR3. All dilutions were prepared in HBSS supplemented with 0.2% BSA. After assembling the binding reaction, the plate was flash-centrifuged at 500 g. Binding reactions were then incubated for 1 hour after which NanoGlo substrate was added (310 times diluted from stock concentration) to a final volume of 13.5 μL. The reaction plate was again pulse-centrifuged to 500 g and the total light intensity was measured for 460 nm wavelength with 80 nm bandwidth and separately for wavelengths ≥610 nm using the PHERAstar-FSX (BMG Labtech, Ortenberg, Germany) with a dual emission filter. The ratio of light intensities (>610 nm over 460 nm) is a measure for the relative binding of CXCL12-A647 to the NanoLuc-ACKR3. The pharmacological data are presented in Appendix 1.

### Chemistry

### General

Unless mentioned otherwise, all reactions were performed under an N₂ atmosphere. All chemicals and solvents were obtained from commercial suppliers (primarily Sigma-Aldrich, Acros Organics, Fluorochem and Combi-Blocks) and used without purification. DCM, DMF, THF and Et₂O were dried by passing through a PureSolv solvent purification system. IUPAC names were adapted from ChemBioDraw Ultra 20.0 (PerkinElmer). Reactions were monitored by thin layer chromatography (Merck Silicagel 60 F254) by visualization under 254 nm lamp. Flash column chromatography was performed with SNAP KP-Sil 50 μm (Biotage) or GraceResolv (Buchi) cartridges on Isolera One with UV-Vis detection (Biotage). Nuclear magnetic resonance (NMR) spectra were determined with a Brücker Avance 500 Ultrashield or a Brucker Avance 600 Ultrashield plus spectrometer. Chemical shifts are reported in parts per million (ppm) against the reference compound using the signal of the residual non-deuterated solvent (e.g. CDCl₃ δ=7.26 ppm (¹H), δ=77.16 ppm (¹³C)). NMR spectra were processed using MestreNova 14.1.0 software. The peak multiplicities are defined as follows: s, singlet; d, doublet; t, triplet; q, quartet; dd, doublet of doublets; ddd, doublet of doublets of doublets; dt, doublet of triplets; dq, doublet of quartets; td, triplet of doublets; tt, triplet of triplets; br, broad signal; m, multiplet; app, apparent. Purity determination was performed with Liquid Chromatography using a Shimadzu LC-20AD liquid chromatography pump system with a Shimadzu SPDM20A photodiode array detector and MS detection with a Shimadzu LCMS-2010EV mass spectrometer operating in both positive and negative ionization mode. A Waters Xbridge C18 column 5 μm 4.6×50 mm was used at 40° C. The mobile phase used was a mixture of A=Water+0.1% HCO₂H and B=acetonitrile (MeCN)+0.1% HCO₂H. The eluent program ‘acidic mode’ used is as follows: flow rate: 1.0 mL/min, start 95% A in a linear gradient to 10% A over 4.5 min, hold 1.5 min at 10% A, in 0.5 min in a linear gradient to 95% A, hold 1.5 min at 95% A, total runtime: 8.0 min. Compound purities were calculated as the percentage peak area of the analyzed compound by UV detection at 254 nm. High-resolution mass spectra (HRMS) were recorded on a Bruker micrOTOF mass spectrometer using ESI in positive ion mode (HRMS). The compounds of the disclosure can easily be synthesized according to techniques known by the person skilled in the art, as shown below.

### List of Abbreviations

ACN Acetonitrile
DCM Dichloromethane
DIPEA Diisopropyl ethyl amine
DMF Dimethylformamide
DMSO Dimethyl Sulfoxide
FA Formic acid
HATU (1-[Bis(dimethylamino)methylene]-1H-1,2,3-triazolo[4,5-b]pyridinium 3-oxide hexafluorophosphate
NMR Nuclear magnetic resonance
NP Normal phase
RP Reverse phase
RT Room temperature
STAB-H Sodium triacetoxyhydroborate
TEA Triethylamine
THE Tetrahydrofuran

### General Procedure A

5-(2,4-difluorophenyl)-N-(piperidin-4-ylmethyl)isoxazole-3-carboxamide hydrochloric acid was suspended in DMF (0.2 M) with AcOH (1.0 eq.) and the respective carbonyl compound (1.4 eq.). After stirring at RT for 15 min, STAB-H (1.4 eq.) was added and the reaction mixture was stirred overnight at RT. In cases where starting material was present additional carbonyl compound and STAB-H were added. The reaction mixture was concentrated in vacuo. The resulting oil was mixed with an aqueous solution of NaOH (2M). The solids were collected by vacuum filtration. In case of the preparation of HCl salt, the solids were dissolved in Et₂O and precipitated with 4M HCl in dioxane. The precipitated HCl salt was collected by vacuum filtration.

### General Procedure B

5-(2,4-difluorophenyl)-N-(piperidin-4-ylmethyl)isoxazole-3-carboxamide hydrochloride was suspended in DCM (0.3 M) and DIPEA (2.5 eq.) was added. The respective acyl chloride or sulfonyl chloride (1.5 eq.) was added and the resulting mixture was stirred overnight at RT. The reaction mixture was washed with an aqueous solution of NaOH (3M) and the aqueous layer was extracted with DCM (twice). The combined organic layers were dried over anhydrous Na₂SO₄, filtered and concentrated in vacuo. The resulting residue was purified using NP column chromatography.

### General Procedure C

5-(2,4-difluorophenyl)-N-(piperidin-4-ylmethyl)isoxazole-3-carboxamide hydrochloride was suspended in DMF (0.3 M) and K₂CO₃ (2.0 eq.) was added. The respective benzyl halide (1.1 eq.) was added and the reaction mixture was stirred overnight at RT. The reaction mixture was poured into an ice-cold aqueous solution of NaOH (2M). Extracted with DCM was performed 3 times. The combined organic layers were dried over anhydrous Na₂SO₄, filtered and concentrated in vacuo. The resulting residue was purified using NP column chromatography.

### General Procedure D

5-(2,4-difluorophenyl)-N-(piperidin-4-ylmethyl)isoxazole-3-carboxamide hydrochloride was suspended in THE (0.3 M) and TEA (1.0 eq.) was added (in some cases AcOH was added). The respective benzaldehyde (1.2 eq.) was added and after stirring for 10 min at RT, STAB-H (2.5 eq.) was added. The resulting mixture was stirred overnight at RT and, if necessary, additional benzaldehyde and STAB-H was added to complete the reaction. MeOH was added to terminate the reaction and the volatiles were removed by in vacuo. The resulting solids were mixed with an aqueous solution of NaOH (2M) and extracted with DCM (3 times). The combined organic layers were dried over anhydrous Na₂SO₄, filtered and concentrated in vacuo. The resulting residue was purified using NP column chromatography.

### General Procedure E

Step i) The corresponding carboxylic acid (1.0 eq.) and HATU (1.5 eq.) were dissolved in DMF (2.0 mL), and DIPEA (1.2 eq.) was added. The resulting mixture was stirred for 30 min at RT. Then, tert-butyl 4-(aminomethyl)piperidine-1-carboxylate (1.2 eq.) dissolved in DMF (0.5 mL) was added. The resulting mixture was stirred for a minimum of 30 min at RT. The mixture was poured into ice-cold satd. aq. Na₂CO₃. Extraction of the suspension with EtOAc and washing of the organic layer with water and brine were performed. The organic layer was dried over anhydrous Na₂SO₄ and filtered. The EtOAc layer was concentrated in vacuo. Step ii): The remaining oil was dissolved in dioxane (2.0 mL) and MeOH (3.0 mL). A 4.0 M solution of HCl in dioxane (10 eq.) was added dropwise, and stirring was continued for 16 h at RT. The solvent was evaporated in vacuo. The residue was used without further purification. Step iii) The residue was suspended in THE (5.0 mL). TEA (1.0 eq.) and cinnamaldehyde (1.2 eq.) were added. After stirring at RT for 10 min, STAB-H (2.5 eq.) was added. The mixture was stirred for 16 h at RT. MeOH was added to terminate the reaction, and the volatiles was removed in vacuo. Reverse-phase column chromatography was used (using a linear gradient of 100% water to 100% ACN, dissolved in DMSO). The solution of product was freeze-dried overnight, and HCl in dioxane (4.0 M) was added to obtain the HCl salt. The dioxane was then evaporated in vacuo and the compound was dried under vacuum.

### General Procedure F

The respective carboxylic acid (1.0 eq.) was suspended in DCM (0.1 M). (COCl)₂ (1.2 eq.) was added and the mixture was activated by adding a few drops of DMF. The mixture was stirred for approximately 15 min at RT. The volatiles were evaporated in vacuo. The residue and the respective amine (1.0 eq.) were mixed with DCM (0.1 M). TEA (2.0 eq.) was added, and the mixture was stirred for 3 h at RT. MeOH was added to terminate the reaction and the volatiles was evaporated in vacuo. The crude product was purified by NP and/or RP column chromatography.

### General Procedure G

The respective amine (1.2 eq.) was dissolved IACN (0.1 M). The respective ethyl ester (1.0 eq.) was added, followed by the addition of 3-chlorophenol (1.0 eq.) and K₂CO₃ (1.0 eq.). The mixture was heated at reflux for 16 h. EtOAc and satd. Aq. NaHCO₃ were added to the mixture. The aqueous layer was extracted with EtOAc. The organic layer was dried over anhydrous Na₂SO₄, filtered and concentrated under reduced pressure. The residue was purified by NP and/or RP column chromatography.

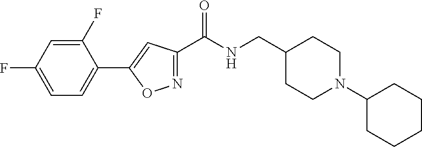 [SMILES: O=C(NCC1CCN(C2CCCCC2)CC1)c1cc(-c2ccc(F)cc2F)on1]

### N-[(1-cyclohexylpiperidin-4-yl)methyl]-5-(2,4-difluorophenyl)-1,2-oxazole-3-carboxamide HCl (1)

This compound was synthesised according to general procedure A. For the first step, 5-(2,4-difluorophenyl)-N-(piperidin-4-ylmethyl)isoxazole-3-carboxamide (200 mg, 0.623 mmol, 1.0 eq.), AcOH (36 μL, 0.62 mmol, 1.0 eq), cyclohexanone (90 μL, 0.872 mmol, 1.4 eq.) and STAB-H (185 mg, 0.872 mmol, 1.4 eq.) were used. The formed HCl salt was triturated with MeOH to obtain the title compound as an off-white solid (26 mg, 9%). LCMS (acidic mode) Rₜ 3.71 min, purity 97%, [M+H]⁺ calculated 404.21, found 404.20. ¹H NMR (500 MHz, Methanol-d₄) δ 8.0 (ddd, J=8.6, 6.2 Hz, 1H), 7.2 (ddd, J=11.3, 8.9, 2.5 Hz, 1H), 7.2 (ddd, J=8.9, 2.5, 1.0 Hz, 1H), 7.1 (d, J=3.5 Hz, 1H), 3.3 (d, J=6.9 Hz, 2H), 3.0-2.9 (m, 2H), 2.3-2.2 (m, 3H), 1.9-1.9 (m, 2H), 1.9-1.8 (m, 4H), 1.7-1.6 (m, 2H), 1.4-1.2 (m, 6H), 1.2-1.1 (m, 1H). ¹³C NMR (126 MHz, MeOD) δ 166.0, 165.8 (dd, J=256.0, 12.0 Hz), 161.2, 161.0 (dd, J=256.5, 12.0 Hz), 160.8, 130.3 (dd, J=10.3, 3.4 Hz), 113.7 (dd, J=22.4, 3.6 Hz), 113.1 (dd, J=12.5, 3.9 Hz), 106.1 (dd, J=26.0 Hz), 103.1 (d, J=10.2 Hz), 65.4, 49.9, 45.9, 37.5, 30.9, 29.4, 27.4, 27.1. HRMS m/z [M+H]⁺ C₁₅H₁₆F₂N₃O₂⁺ calculated 404.2144, found 404.2151.

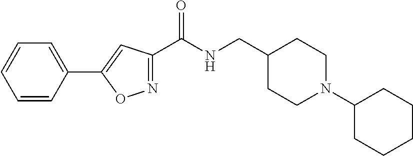 [SMILES: O=C(NCC1CCN(C2CCCCC2)CC1)c1cc(-c2ccccc2)on1]

### N-[(1-cyclohexylpiperidin-4-yl)methyl]-5-phenyl-1,2-oxazole-3-carboxamide (2)

This compound was synthesised according to general procedure A. For the first step, 5-phenyl-N-(piperidin-4-ylmethyl)isoxazole-3-carboxamide (177 mg, 0.623 mmol, 1.0 eq.), AcOH (36 μL, 0.62 mmol, 1.0 eq), cyclohexanone (90 μL, 0.872 mmol, 1.4 eq.) and STAB-H (185 mg, 0.872 mmol, 1.4 eq.) were used. The crude product was purified by NP column chromatography (using a linear gradient of 10%-50% EtOAc:MeOH:TEA 90:5:5 in cyclohexane) to obtain the title compound as an off-white solid (150 mg, 66%). LCMS (acidic mode) Rₜ min 3.57, purity >99%, [M+H]⁺ calculated 368.23, found 368.25. ¹H NMR (500 MHz, Methanol-d₄) δ 7.89-7.86 (m, 2H), 7.55-7.50 (m, 3H), 7.08 (s, 1H), 3.29 (d, J=6.7 Hz, 2H), 3.00 (dt, J=12.2, 3.5 Hz, 2H), 2.41-2.31 (m, 3H), 1.97-1.90 (m, 2H), 1.85-1.78 (m, 4H), 1.72-1.62 (m, 2H), 1.39-1.23 (m, 6H), 1.19-1.08 (m, 1H). ¹³C NMR (126 MHz, MeOD) δ 172.8, 161.5, 160.7, 131.9, 130.3, 128.2, 126.9, 99.9, 65.5, 49.9, 45.8, 37.4, 30.7, 29.3, 27.3, 27.1. HRMS m/z [M+H]⁺ C₂₂H₃₀N₃O₂⁺ calculated 368.2333, found 368.2348.

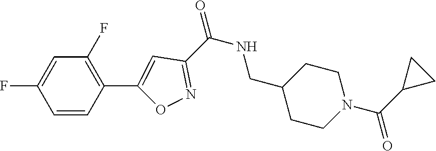 [SMILES: O=C(NCC1CCN(C(=O)C2CC2)CC1)c1cc(-c2ccc(F)cc2F)on1]

### N-((1-(cyclopropanecarbonyl)piperidin-4-yl)methyl)-5-(2,4-difluorophenyl)isoxazole-3-carboxamide (7)

This compound was synthesised according to general procedure B. 5-(2,4-difluorophenyl)-N-(piperidin-4-ylmethyl)isoxazole-3-carboxamide hydrochloride (200 mg, 0.559 mmol, 1.0 eq.), DIPEA (243 μL, 1.40 mmol, 2.5 eq) and cyclopropylcarbonyl chloride (76 μL, 0.84 mmol, 1.5 eq.) were used.

The crude product was purified by NP column chromatography (using a linear gradient of 50% EtOAc/cyclohexane to 100% EtOAc) to obtain the title compound as an off-white solid (167 mg, 77%). LC/MS (acidic mode) Rₜ 4.19 min, purity >99%, [M+H]⁺ calculated 390.16, found 390.20. ¹H NMR (500 MHz, DMSO-d6) δ 8.96 (t, J=6.0 Hz, 1H), 8.07 (ddd, J=8.7, 8.7, 6.4 Hz, 1H), 7.59 (ddd, J=11.6, 9.2, 2.6 Hz, 1H), 7.34 (ddd, J=8.6, 8.6, 2.5 Hz, 1H), 7.17 (d, J=3.0 Hz, 1H), 4.41-4.20 (m, 2H), 3.26-3.13 (m, 2H), 3.13-3.01 (m, 1H), 2.63-2.54 (m, 1H), 2.00-1.93 (m, 1H), 1.93-1.82 (m, 1H), 1.82-1.63 (m, 2H), 1.21-0.97 (m, 2H), 0.77-0.63 (m, 4H). ¹³C NMR (126 MHz, DMSO-d6) δ 170.7, 164.0 (d, J=3.5 Hz), 163.7 (dd, J=251.7, 13.3 Hz), 159.6, 159.0 (dd, J=255.2, 12.8 Hz), 158.3, 129.6 (d, J=10.4 Hz), 113.0 (dd, J=22.1, 3.5 Hz), 111.5 (d, J=12.4 Hz), 105.5 (dd, J=26.0, 26.0 Hz), 102.5 (d, J=8.3 Hz), 44.8, 44.2, 41.5, 35.9, 30.4, 29.4, 10.3, 6.8. Rotamers are observed. HRMS m/z [M+Na]⁺ C₂₀H₂₁F₂N₃NaO₃⁺ calculated 412.1443, found 412.1432.

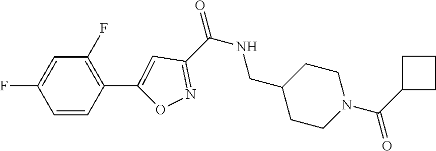 [SMILES: O=C(NCC1CCN(C(=O)C2CCC2)CC1)c1cc(-c2ccc(F)cc2F)on1]

### N-((1-(cyclobutanecarbonyl)piperidin-4-yl)methyl)-5-(2,4-difluorophenyl)isoxazole-3-carboxamide (8)

This compound was synthesised according to general procedure B. 5-(2,4-difluorophenyl)-N-(piperidin-4-ylmethyl)isoxazole-3-carboxamide hydrochloride (200 mg, 0.559 mmol, 1.0 eq.), DIPEA (243 μL, 1.40 mmol, 2.5 eq) and cyclobutylcarbonyl chloride (96 μL, 0.84 mmol, 1.5 eq.) were used. The crude product was purified by NP column chromatography (using a linear gradient of 50% EtOAc/cyclohexane to 100% EtOAc) to obtain the title compound as an off-white solid (124 mg, 55%). LC/MS (acidic mode) Rₜ 4.42 min, purity >99%, [M+H]⁺ calculated 404.18, found 404.20. ¹H NMR (500 MHz, DMSO-d6) δ 8.92 (t, J=6.0 Hz, 1H), 8.05 (ddd, J=8.7, 8.7, 6.4 Hz, 1H), 7.57 (ddd, J=11.5, 9.3, 2.6 Hz, 1H), 7.33 (ddd, J=8.6, 8.6, 2.5 Hz, 1H), 7.15 (d, J=2.9 Hz, 1H), 4.38-4.29 (m, 1H), 3.71-3.63 (m, 1H), 3.32-3.26 (m, 1H), 3.21-3.10 (m, 2H), 2.92-2.83 (m, 1H), 2.55-2.46 (m, 1H), 2.19-2.01 (m, 4H), 1.93-1.75 (m, 2H), 1.75-1.60 (m, 3H), 1.07-0.92 (m, 2H). ¹³C NMR (126 MHz, DMSO-d6) δ 171.7, 164.0 (d, J=2.1 Hz), 163.7 (dd, J=251.9, 12.4 Hz), 159.6, 159.0 (dd, J=255.3, 12.7 Hz), 158.3, 129.6 (dd, J=10.4, 3.2 Hz), 113.0 (dd, J=21.9, 3.2 Hz), 111.5 (dd, J=12.4, 3.8 Hz), 105.5 (dd, J=26.0, 26.0 Hz), 102.5 (d, J=8.4 Hz), 44.2 (d, J=3.5 Hz), 41.0, 36.4, 35.8, 30.3, 29.4, 24.7, 24.5, 17.4. Rotamers are observed. HRMS m/z [M+Na]⁺ C₂₁H₂₃F₂N₃NaO₃⁺ calculated 426.1600, found 426.1600.

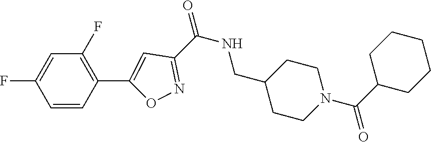 [SMILES: O=C(NCC1CCN(C(=O)C2CCCCC2)CC1)c1cc(-c2ccc(F)cc2F)on1]

### N-((1-(cyclohexanecarbonyl)piperidin-4-yl)methyl)-5-(2,4-difluorophenyl)isoxazole-3-carboxamide (9)

This compound was synthesised according to general procedure B. 5-(2,4-difluorophenyl)-N-(piperidin-4-ylmethyl)isoxazole-3-carboxamide hydrochloride (200 mg, 0.559 mmol, 1.0 eq.), DIPEA (243 μL, 1.40 mmol, 2.5 eq) and isobutyryl chloride (112 μL, 0.838 mmol, 1.5 eq.) were used. The crude product was purified by NP column chromatography (using a linear gradient of 50% EtOAc/cyclohexane to 100% EtOAc) to obtain the title compound as an off-white solid (124 mg, 55%). LC/MS (acidic mode) Rₜ 4.42 min, purity >99%, [M+H]⁺ calculated 404.18, found 404.20. ¹H NMR (500 MHz, DMSO-d6) δ 8.93 (t, J=6.0 Hz, 1H), 8.04 (ddd, J=8.7, 8.6, 6.3 Hz, 1H), 7.59-7.53 (m, 1H), 7.32 (ddd, J=8.6, 8.5, 2.5 Hz, 1H), 7.14 (d, J=2.8 Hz, 1H), 4.41-4.31 (m, 1H), 3.96-3.87 (m, 1H), 3.23-3.10 (m, 2H), 3.01-2.90 (m, 1H), 2.58-2.52 (m, 1H), 2.48-2.44 (m, 1H), 1.87-1.76 (m, 1H), 1.76-1.56 (m, 7H), 1.36-1.20 (m, 4H), 1.20-1.02 (m, 2H), 1.02-0.89 (m, 1H). ¹³C NMR (126 MHz, DMSO-d6) δ 173.7, 164.5, 164.1 (dd, J=252.0, 12.5 Hz), 160.0, 159.5 (dd, J=255.3, 12.9 Hz), 158.8, 130.1 (dd, J=10.4, 3.4 Hz), 113.5 (dd, J=22.1, 3.5 Hz), 111.9 (dd, J=12.5, 3.9 Hz), 105.9 (dd, J=26.0, 26.0 Hz), 102.9 (d, J=8.5 Hz), 45.1, 44.6, 41.3, 39.6, 36.3, 31.0, 29.8, 29.6, 26.1, 25.7. Rotamers are observed. HRMS m/z [M+Na]⁺ C₂₃H₂₇F₂N₃NaO₃⁺ calculated 454.1913, found 454.1922.

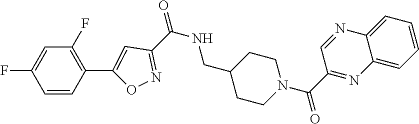 [SMILES: O=C(NCC1CCN(C(=O)c2cnc3ccccc3n2)CC1)c1cc(-c2ccc(F)cc2F)on1]

### 5-(2,4-difluorophenyl)-N-((1-(quinoxaline-2-carbonyl)piperidin-4-yl)methyl)isoxazole-3-carboxamide (12)

This compound was synthesised according to general procedure B. 5-(2,4-difluorophenyl)-N-(piperidin-4-ylmethyl)isoxazole-3-carboxamide hydrochloride (200 mg, 0.559 mmol, 1.0 eq.), DIPEA (243 μL, 1.40 mmol, 2.5 eq) and quinoxaline-2-carbonyl chloride (162 mg, 0.838 mmol, 1.5 eq.) were used. The crude product was purified by NP column chromatography (using a linear gradient of 50% EtOAc/cyclohexane to 100% EtOAc) to obtain the title compound as an off-white solid (165 mg, 62%). LC/MS (acidic mode) Rₜ 4.42 min, purity >99%, [M+H]⁺ calculated 478.17, found 478.25. ¹H NMR (500 MHz, DMSO-d6) δ 9.09 (s, 1H), 8.98 (t, J=6.0 Hz, 1H), 8.17-8.13 (m, 1H), 8.13-8.08 (m, 1H), 8.05 (ddd, J=8.7, 8.7, 6.3 Hz, 1H), 7.96-7.91 (m, 2H), 7.57 (ddd, J=11.5, 9.2, 2.5 Hz, 1H), 7.32 (ddd, J=8.5, 8.4, 2.5 Hz, 1H), 7.15 (d, J=2.9 Hz, 1H), 4.59-4.52 (m, 1H), 3.85-3.79 (m, 1H), 3.27-3.20 (m, 2H), 3.19-3.10 (m, 1H), 2.95-2.87 (m, 1H), 1.98-1.89 (m, 1H), 1.89-1.82 (m, 1H), 1.70-1.63 (m, 1H), 1.35-1.19 (m, 2H). ¹³C NMR (126 MHz, DMSO-d6) δ 164.7, 164.0 (d, J=2.4 Hz), 163.3 (dd, J=252.2, 12.7 Hz), 159.6, 159.0 (dd, J=255.3, 12.7 Hz), 158.3, 149.2, 144.7, 141.6, 139.9, 131.2, 131.0, 129.6 (dd, J=10.4, 3.3 Hz), 129.3, 129.0, 113.0 (dd, J=22.1, 3.5 Hz), 111.5 (dd, J=12.5, 3.9 Hz), 105.5 (dd, J=25.9, 25.9 Hz), 102.5 (d, J=8.5 Hz), 46.5, 44.1, 41.5, 35.6, 30.1, 29.3. Rotamers are observed. HRMS m/z [M+Na]⁺ C₂₅H₂₁F₂N₅NaO₃⁺ calculated 500.15050, found 500.1480.

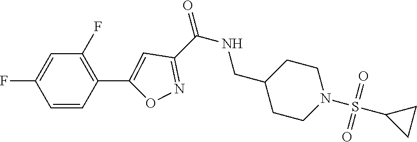 [SMILES: O=C(NCC1CCN(S(=O)(=O)C2CC2)CC1)c1cc(-c2ccc(F)cc2F)on1]

### N-((1-(cyclopropylsulfonyl)piperidin-4-yl)methyl)-5-(2,4-difluorophenyl)isoxazole-3-carboxamide (14)

This compound was synthesised according to general procedure B. 5-(2,4-difluorophenyl)-N-(piperidin-4-ylmethyl)isoxazole-3-carboxamide hydrochloride (200 mg, 0.559 mmol, 1.0 eq.), DIPEA (243 μL, 1.40 mmol, 2.5 eq) and cyclopropanesulfonyl chloride (85 μL, 0.84 mmol, 1.5 eq.) were used. The crude product was purified by NP column chromatography (using a linear gradient of 50% EtOAc/cyclohexane to 100% EtOAc) to obtain the title compound as an off-white solid (200 mg, 84%). LC/MS (acidic mode) Rₜ 4.39 min, purity 99%, [M+H]⁺ calculated 426.13, found 426.20. ¹H NMR (600 MHz, DMSO-d6) δ 8.93 (t, J=6.0 Hz, 1H), 8.05 (ddd, J=8.6, 8.6, 6.2 Hz, 1H), 7.56 (ddd, J=11.5, 9.1, 2.5 Hz, 1H), 7.32 (ddd, J=8.6, 8.5, 2.6 Hz, 1H), 7.15 (d, J=2.9 Hz, 1H), 3.64-3.55 (m, 2H), 3.20 (dd, J=6.3, 6.3 Hz, 2H), 2.83-2.74 (m, 2H), 2.58-2.52 (m, 1H), 1.81-1.68 (m, 3H), 1.26-1.15 (m, 2H), 1.00-0.84 (m, 4H). ¹³C NMR (151 MHz, DMSO-d6) δ 164.0 (d, J=3.1 Hz), 163.7 (dd, J=252.1, 12.4 Hz), 159.6, 159.0 (dd, J=255.3, 12.8 Hz), 158.3, 129.6 (dd, J=10.3, 3.3 Hz), 113.0 (dd, J=22.1, 3.4 Hz), 111.5 (dd, J=12.4, 4.1 Hz), 105.5 (dd, J=26.0, 26.0 Hz), 102.5 (d, J=8.4 Hz), 45.6, 44.0, 34.9, 29.1, 25.2, 3.9. HRMS m/z [M+Na]⁺ C₁₉H₂₁F₂N₃NaO₄S⁺ calculated 448.1113 found 448.1094.

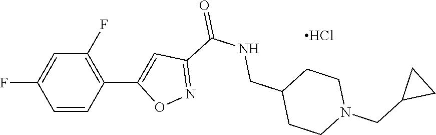 [SMILES: O=C(NCC1CCN(CC2CC2)CC1)c1cc(-c2ccc(F)cc2F)on1 ; Cl]

### N-((1-(cyclopentylmethyl)piperidin-4-yl)methyl)-5-(2,4-difluorophenyl)isoxazole-3-carboxamide (16)

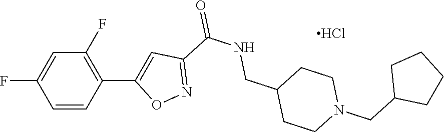 [SMILES: O=C(NCC1CCN(CC2CCCC2)CC1)c1cc(-c2ccc(F)cc2F)on1 ; Cl]

This compound was synthesised according to general procedure A. 5-(2,4-difluorophenyl)-N-(piperidin-4-ylmethyl)isoxazole-3-carboxamide (200 mg, 0.623 mmol, 1.0 eq.), AcOH (36 μL, 0.62 mmol, 1.0 eq), cyclopentanecarboxaldehyde (93 μL, 0.87 mmol, 1.4 eq.) and STAB-H (185 mg, 0.872 mmol, 1.4 eq.) were used. The formed HCl salt was triturated with MeOH to obtain the title compound as an off-white solid (222 mg, 81%). LC/MS (acidic mode) Rₜ 3.82 min, purity >98%, [M+H]⁺ calculated 404.21, found 404.20. ¹H NMR (500 MHz, DMSO-d6) δ 10.44 (d, J=71.9 Hz, 1H), 9.26-9.00 (m, 1H), 8.06 (ddd, J=8.7, 8.7, 6.4 Hz, 1H), 7.59 (ddd, J=11.5, 9.2, 2.5 Hz, 1H), 7.38-7.31 (m, 1H), 7.24 (d, J=2.9 Hz, 1H), 3.46 (s, 2H), 3.19 (dd, J=6.4, 6.4 Hz, 2H), 3.00 (d, J=7.0 Hz, 2H), 2.90 (d, J=10.6 Hz, 2H), 2.30-2.26 (m, 1H), 2.00-1.77 (m, 5H), 1.77-1.64 (m, 2H), 1.64-1.55 (m, 2H), 1.55-1.43 (m, 2H), 1.33-1.20 (m, 2H). ¹³C NMR (126 MHz, DMSO-d6) δ 164.1 (d, J=2.7 Hz), 163.7 (dd, J=252.0, 12.3 Hz), 159.6, 159.0 (dd, J=255.3, 12.8 Hz), 158.4, 129.7 (dd, J=10.4, 3.3 Hz), 113.1 (dd, J=22.1, 3.5 Hz), 111.5 (dd, J=12.3, 4.1 Hz), 105.5 (dd, J=25.9, 25.9 Hz), 102.5 (d, J=8.5 Hz), 61.1, 51.7, 43.8, 34.4, 34.0, 31.2, 26.6, 24.7. HRMS m/z [M+H]⁺ C₂₂H₂₈F₂N₃O₂⁺ calculated 404.2144, found 404.2136.

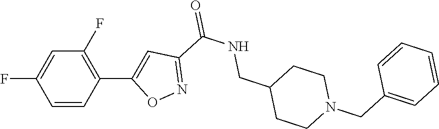 [SMILES: O=C(NCC1CCN(Cc2ccccc2)CC1)c1cc(-c2ccc(F)cc2F)on1]

### N-((1-benzylpiperidin-4-yl)methyl)-5-(2,4-difluorophenyl)isoxazole-3-carboxamide (17)

5-(2,4-difluorophenyl)-N-(piperidin-4-ylmethyl)isoxazole-3-carboxamide (200 mg, 0.623 mmol, 1.0 eq.) was suspended in DMF (0.2 M) and AcOH (36 μL, 0.62 mmol, 1.0 eq) and benzaldehyde (88 μL, 0.87 mmol, 1.4 eq.) were added. The reaction was stirred for 15 min followed by the addition of STAB-H (185 mg, 0.872 mmol, 1.4 eq.). The resulting suspension was stirred overnight at RT. The reaction mixture was concentrated in vacuo and the resulting oil was mixed with an aqueous solution of NaOH (2M). The formed solids were collected by vacuum filtration and triturated with MeOH to obtain the title compound as an off-white solid (17 mg, 6%). LC/MS (acidic mode) Rₜ 3.79 min, purity 94%, [M+H]⁺ calculated 418.23, found 418.25. ¹H NMR (500 MHz, DMSO-d6) δ 8.92-8.83 (m, 1H), 8.09-7.99 (m, 1H), 7.62-7.51 (m, 1H), 7.35-7.25 (m, 5H), 7.25-7.20 (m, 1H), 7.16-7.12 (m, 1H), 3.50-3.39 (m, 2H), 3.16 (dd, J=6.5, 6.5 Hz, 2H), 2.84-2.71 (m, 2H), 1.94-1.81 (m, 2H), 1.69-1.59 (m, 2H), 1.59-1.49 (m, 1H), 1.26-1.09 (m, 2H). ¹³C NMR (126 MHz, DMSO-d6) δ 164.0, 163.6 (dd, J=252.3, 12.7 Hz), 159.6, 159.0 (dd, J=255.8, 13.7 Hz), 158.2, 138.7, 129.6 (dd, J=10.0, 3.1 Hz), 128.7, 128.1, 126.8, 113.0 (d, J=23.5 Hz), 111.9-111.2 (m), 105.5 (dd, J=26.0, 26.0 Hz), 102.5 (d, J=8.5 Hz), 62.4, 52.9, 44.4, 35.6, 29.7. HRMS m/z [M+H]⁺ C₂₃H₂₄F₂N₃O₂⁺ calculated 412.1831, found 412.1819.

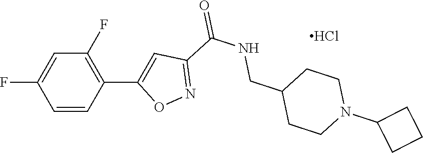 [SMILES: O=C(NCC1CCN(C2CCC2)CC1)c1cc(-c2ccc(F)cc2F)on1 ; Cl]

### N-((1-cyclobutylpiperidin-4-yl)methyl)-5-(2,4-difluorophenyl)isoxazole-3-carboxamide hydrochloride (18)

This compound was synthesised according to general procedure A. 5-(2,4-difluorophenyl)-N-(piperidin-4-ylmethyl)isoxazole-3-carboxamide (200 mg, 0.623 mmol, 1.0 eq.), AcOH (36 μL, 0.62 mmol, 1.0 eq), cyclobutanone (65 μL, 0.87 mmol, 1.4 eq.) and STAB-H (185 mg, 0.872 mmol, 1.4 eq.) were used. The formed HCl salt was triturated with MeOH to obtain the title compound as an off-white solid (169 mg, 66%). LC/MS (acidic mode) Rₜ 3.48 min, purity >98%, [M+H]⁺ calculated 376.18, found 376.20. ¹H NMR (500 MHz, DMSO-d6) δ 11.49-11.22 (m, 1H), 9.12 (t, J=6.1 Hz, 1H), 8.05 (ddd, J=8.7, 8.7, 6.3 Hz, 1H), 7.58 (ddd, J=11.6, 9.3, 2.6 Hz, 1H), 7.33 (ddd, J=8.6, 8.5, 2.2 Hz, 1H), 7.22 (d, J=2.9 Hz, 1H), 3.58-3.47 (m, 1H), 3.30-3.23 (m, 2H), 3.16 (dd, J=6.4, 6.4 Hz, 2H), 2.71-2.61 (m, 2H), 2.48-2.38 (m, 2H), 2.15-2.06 (m, 2H), 1.84-1.77 (m, 3H), 1.75-1.55 (m, 4H). ¹³C NMR (126 MHz, DMSO-d6) δ 164.1, 163.7 (dd, J=252.3, 12.4 Hz), 159.5, 159.0 (dd, J=255.4, 12.8 Hz), 158.3, 129.7 (dd, J=10.4, 3.5 Hz), 113.0 (dd, J=22.2, 3.5 Hz), 111.5 (dd, J=12.3, 3.5 Hz), 105.5 (dd, J=25.9, 25.9 Hz), 102.5 (d, J=8.2 Hz), 58.2, 48.0, 43.8, 33.6, 26.2, 24.7, 13.3. HRMS m/z [M+H]⁺ C₂₀H₂₄F₂N₃O₂⁺ calculated 376.1831, found 376.1829.

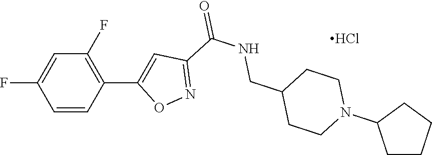 [SMILES: O=C(NCC1CCN(C2CCCC2)CC1)c1cc(-c2ccc(F)cc2F)on1 ; Cl]

### N-((1-cyclopentylpiperidin-4-yl)methyl)-5-(2,4-difluorophenyl)isoxazole-3-carboxamide hydrochloride (19)

This compound was synthesised according to general procedure A. 5-(2,4-difluorophenyl)-N-(piperidin-4-ylmethyl)isoxazole-3-carboxamide (200 mg, 0.623 mmol, 1.0 eq.), AcOH (36 μL, 0.62 mmol, 1.0 eq), cyclopentanone (77 μL, 0.87 mmol, 1.4 eq.) and STAB-H (185 mg, 0.872 mmol, 1.4 eq.) were used. Additional cyclopentanone (77 μL, 0.87 mmol, 1.4 eq.) and STAB-H (185 mg, 0.872 mmol, 1.4 eq.) were added two times over 2 days to complete the reaction. The formed HCl salt was triturated with MeOH to obtain the title compound as an off-white solid (114 mg, 43%). LC/MS (acidic mode) Rₜ 3.88 min, purity 95%, [M+H]⁺ calculated 390.20, found 390.15. ¹H NMR (500 MHz, DMSO-d6) δ 10.84 (s, 1H), 9.09 (t, J=6.0 Hz, 1H), 8.05 (ddd, J=8.7, 8.6, 6.3 Hz, 1H), 7.58 (ddd, J=11.6, 9.2, 2.6 Hz, 1H), 7.33 (ddd, J=8.5, 8.3, 2.1 Hz, 1H), 7.20 (d, J=2.9 Hz, 1H), 3.48-3.42 (m, 2H), 3.36-3.31 (m, 2H), 3.16 (dd, J=6.3, 6.3 Hz, 2H), 2.89-2.79 (m, 2H), 2.00-1.93 (m, 2H), 1.88-1.76 (m, 4H), 1.74-1.56 (m, 4H), 1.56-1.46 (m, 2H). ¹³C NMR (126 MHz, DMSO-d6) δ 164.1 (d, J=2.6 Hz), 163.7 (dd, J=252.2, 12.4 Hz), 159.6, 159.0 (dd, J=255.5, 12.9 Hz), 158.4, 129.7, 113.0 (dd, J=22.1, 3.5 Hz), 111.5 (dd, J=12.3, 3.7 Hz), 105.5 (dd, J=26.0, 26.0 Hz), 102.5 (d, J=7.7 Hz), 66.6, 50.6, 43.7, 33.6, 27.5, 26.8, 23.3. HRMS m/z [M+H]⁺ C₂₁H₂₆F₂N₃O₂⁺ calculated 390.1988, found 390.2003.

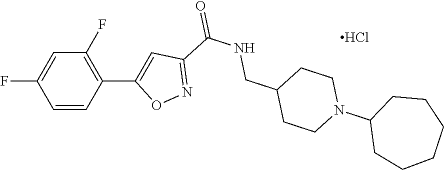 [SMILES: O=C(NCC1CCN(C2CCCCCC2)CC1)c1cc(-c2ccc(F)cc2F)on1 ; Cl]

### N-((1-cycloheptylpiperidin-4-yl)methyl)-5-(2,4-difluorophenyl)isoxazole-3-carboxamide hydrochloride (20)

This compound was synthesised according to general procedure A. For the first step, 5-(2,4-difluorophenyl)-N-(piperidin-4-ylmethyl)isoxazole-3-carboxamide (200 mg, 0.623 mmol, 1.0 eq.), AcOH (36 μL, 0.62 mmol, 1.0 eq), cycloheptanone (103 μL, 0.872 mmol, 1.4 eq.) and STAB-H (185 mg, 0.872 mmol, 1.4 eq.) were used. The formed HCl salt was triturated with MeOH to obtain the title compound as an off-white solid (26 mg, 9%). LC/MS (acidic mode) Rₜ 3.92 min, purity 99%, [M+H]⁺ calculated 418.23, found 418.25. ¹H NMR (500 MHz, DMSO-d6) δ 10.51-10.23 (m, 1H), 9.07 (t, J=6.0 Hz, 1H), 8.05 (ddd, J=8.7, 8.7, 6.4 Hz, 1H), 7.58 (ddd, J=11.5, 9.2, 2.5 Hz, 1H), 7.33 (ddd, J=8.6, 8.4, 2.2 Hz, 1H), 7.19 (d, J=2.9 Hz, 1H), 3.32-3.20 (m, 3H), 3.16 (dd, J=6.4, 6.4 Hz, 2H), 3.02-2.90 (m, 2H), 2.15-2.06 (m, 2H), 1.92-1.84 (m, 1H), 1.85-1.78 (m, 2H), 1.72-1.62 (m, 4H), 1.62-1.55 (m, 2H), 1.53-1.36 (m, 6H). ¹³C NMR (126 MHz, DMSO-d6) δ 164.0 (d, J=2.7 Hz), 163.6 (dd, J=252.2, 12.5 Hz), 159.6, 159.0 (dd, J=255.4, 12.8 Hz), 158.3, 129.6 (dd, J=10.3, 3.3 Hz), 113.0 (dd, J=22.0, 3.5 Hz), 111.5 (dd, J=12.4, 3.8 Hz), 105.5 (dd, J=26.0, 26.0 Hz), 102.5 (d, J=8.4 Hz), 65.5, 47.5, 43.8, 33.7, 27.5, 26.9, 26.7, 24.1. HRMS m/z [M+H]⁺ C₂₃H₃₀F₂N₃O₂⁺ calculated 418.2301, found 418.2302.

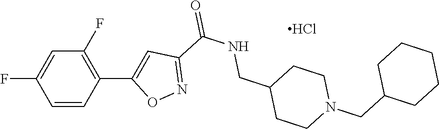 [SMILES: O=C(NCC1CCN(CC2CCCCC2)CC1)c1cc(-c2ccc(F)cc2F)on1 ; Cl]

### N-((1-(cyclohexylmethyl)piperidin-4-yl)methyl)-5-(2,4-difluorophenyl)isoxazole-3-carboxamide hydrochloride (22)

This compound was synthesised according to general procedure A. 5-(2,4-difluorophenyl)-N-(piperidin-4-ylmethyl)isoxazole-3-carboxamide (200 mg, 0.623 mmol, 1.0 eq.), AcOH (36 μL, 0.62 mmol, 1.0 eq), cyclohexanecarboxaldehyde (106 μL, 0.872 mmol, 1.4 eq.) and STAB-H (185 mg, 0.872 mmol, 1.4 eq.) was used. The HCl salt was purified by RP column chromatography (using a linear gradient of 30-50% ACN/H₂O with 0.1% FA). The product fractions were combined, basified with an aqueous solution of NaOH (2M) and extracted with DCM (3 times). The organic layer was dried over anhydrous Na₂SO₄, filtered and an excess of 4M HCl in dioxane was added. The resulting mixture was concentrated in vacuo to obtain the title compound as an off-white solid (187 mg, 72%). LC/MS (acidic mode) Rₜ 3.91 min, purity >99%, [M+H]⁺ calculated 418.23, found 418.25. ¹H NMR (500 MHz, DMSO-d6) δ 10.53-10.24 (m, 1H), 9.12 (t, J=6.0 Hz, 1H), 8.04 (ddd, J=8.7, 8.6, 6.3 Hz, 1H), 7.57 (ddd, J=11.5, 9.1, 2.6 Hz, 1H), 7.33 (ddd, J=8.5, 8.5, 2.5 Hz, 1H), 7.22 (d, J=2.9 Hz, 1H), 3.49-3.43 (m, 2H), 3.19-3.10 (m, 2H), 2.98-2.77 (m, 4H), 1.98-1.69 (m, 8H), 1.69-1.54 (m, 3H), 1.27-1.16 (m, 2H), 1.16-1.05 (m, 1H), 0.99-0.86 (m, 2H). ¹³C NMR (126 MHz, DMSO-d6) δ 164.0 (d, J=2.8 Hz), 163.7 (dd, J=252.0, 12.3 Hz), 159.6, 159.0 (dd, J=255.4, 12.9 Hz), 158.4, 129.6 (dd, J=10.4, 3.2 Hz), 113.0 (dd, J=22.1, 3.5 Hz), 111.5 (dd, J=12.5, 3.9 Hz), 105.5 (dd, J=25.9, 25.9 Hz), 102.5 (d, J=8.2 Hz), 62.2, 52.0, 43.8, 33.5, 32.1, 31.0, 26.4, 25.5, 25.1. HRMS m/z [M+H]⁺ C₂₃H₃₀F₂N₃O₂⁺ calculated 418.2301, found 418.2307.

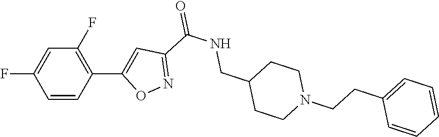 [SMILES: O=C(NCC1CCN(CCc2ccccc2)CC1)c1cc(-c2ccc(F)cc2F)on1]

### 5-(2,4-difluorophenyl)-N-((1-phenethylpiperidin-4-yl)methyl)isoxazole-3-carboxamide (23)

5-(2,4-difluorophenyl)-N-(piperidin-4-ylmethyl)isoxazole-3-carboxamide (200 mg, 0.623 mmol, 1.0 eq.) was suspended in DMF (2.8 mL) that contained AcOH (36 μL, 0.62 mmol, 1.0 eq.) and 2-phenylacetaldehyde (97 μL, 0.87 mmol, 1.4 eq.). The mixture was stirred at RT for 10 min. STAB-H (185 mg, 0.871 mmol, 1.4 eq.) were added and the reaction mixture was stirred overnight. The DMF was removed in vacuo and the resulting crude product was suspended in an aqueous solution of NaOH (2M). The formed solids were collected by vacuum filtration. The crude product was dissolved in dioxane and converted to the HCl salt by addition of 4M HCl in dioxane. The resulting HCl salt was purified by RP column chromatography (using a linear gradient of 10% ACN/H₂O with 0.1% FA to 100% ACN). The product fractions were combined, basified with an aqueous solution of NaOH (2M) and extracted three times with DCM. The combined DCM layers were dried over anhydrous Na₂SO₄, filtered and concentrated in vacuo, yielding the title compound as an off-white solid (178 mg, 67%). LC/MS (acidic mode) Rₜ 3.90 min, purity >99%, [M+H]⁺ calculated 426.20, found 426.25. ¹H NMR (500 MHz, DMSO-d6) δ 11.33-11.14 (m, 1H), 9.13 (t, J=5.9 Hz, 1H), 8.08-8.01 (m, 1H), 7.61-7.53 (m, 1H), 7.32 (dd, J=9.8, 5.0 Hz, 3H), 7.26 (d, J=7.7 Hz, 2H), 7.24-7.21 (m, 2H), 3.57-3.49 (m, 2H), 3.24-3.14 (m, 4H), 3.14-3.07 (m, 2H), 3.00-2.85 (m, 2H), 1.92-1.81 (m, 3H), 1.71-1.59 (m, 2H). ¹³C NMR 126 MHz, DMSO-d6) δ 164.0 (d, J=2.7 Hz), 163.6 (dd, J=252.2, 12.5 Hz), 159.5, 159.0 (dd, J=255.2, 12.8 Hz), 158.3, 137.4, 129.6 (dd, J=10.3, 3.3 Hz), 128.7, 128.6, 126.7, 113.0 (dd, J=22.2, 3.5 Hz), 111.5 (dd, J=12.3, 3.6 Hz), 105.5 (dd, J=25.8, 25.8 Hz), 102.5 (d, J=8.4 Hz), 56.6, 51.3, 43.7, 33.6, 29.3, 26.8. HRMS m/z [M+H]⁺ C₂₄H₂₆F₂N₃O₂⁺ calculated 426.1988, found 426.1995.

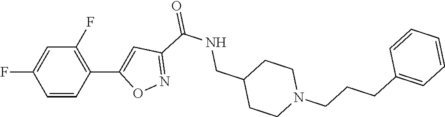 [SMILES: O=C(NCC1CCN(CCCc2ccccc2)CC1)c1cc(-c2ccc(F)cc2F)on1]

### 5-(2,4-difluorophenyl)-N-((1-(3-phenylpropyl)piperidin-4-yl)methyl)isoxazole-3-carboxamide (24)

5-(2,4-difluorophenyl)-N-(piperidin-4-ylmethyl)isoxazole-3-carboxamide hydrochloride (100 mg, 0.279 mmol, 1.0 eq.) was suspended in DMF (2.8 mL) and TEA (39 μL, 0.28 mmol, 1.0 eq.), AcOH (16 μL, 0.28 mmol, 1.0 eq.) and hydrocinnamaldehyde (44 μL, 0.34 mmol, 1.2 eq.) were added. After stirring for 10 min at RT, STAB-H (71 mg, 0.34 mmol, 1.2 eq.) was added and the reaction mixture was stirred overnight at RT. Additional hydrocinnamaldehyde (44 μL, 0.34 mmol, 1.2 eq.) and STAB-H (71 mg, 0.34 mmol, 1.2 eq.) were added to complete the reaction. MeOH was added to terminate the reaction and the volatiles were removed by in vacuo. The resulting solids were suspended in an aqueous solution of NaOH (2M) and extracted with DCM (3 times). The combined organic layers were dried over anhydrous Na₂SO₄, filtered and concentrated in vacuo. The crude product was purified by NP column chromatography (using a linear gradient of 10-60% (EtOAc:MeOH:TEA, 90:5:5, v:v:v %)/cyclohexane) to obtain the title compound as an off-white solid (66 mg, 54%). LC/MS (acidic mode) Rₜ 4.33 min, purity 99%, [M+H]⁺ calculated 440.21, found 440.25. ¹H NMR (500 MHz, DMSO-d6) δ 8.87 (t, J=6.0 Hz, 1H), 8.05 (ddd, J=8.7, 8.7, 6.3 Hz, 1H), 7.58 (ddd, J=11.5, 9.2, 2.5 Hz, 1H), 7.36-7.30 (m, 1H), 7.26 (t, J=7.5 Hz, 2H), 7.20-7.17 (m, 2H), 7.16-7.12 (m, 2H), 3.15 (dd, J=6.4, 6.4 Hz, 2H), 2.86-2.77 (m, 2H), 2.60-2.53 (m, 2H), 2.26-2.20 (m, 2H), 1.83-1.75 (m, 2H), 1.75-1.66 (m, 2H), 1.66-1.59 (m, 2H), 1.59-1.49 (m, 1H), 1.23-1.10 (m, 2H). ¹³C NMR (126 MHz, DMSO-d6) δ 164.2-163.9 (m), 163.6 (dd, J=252.0, 12.3 Hz), 159.6, 159.0 (dd, J=255.2, 12.7 Hz), 158.2, 142.2, 129.6 (dd, J=10.4, 3.3 Hz), 129.0, 128.6-127.9 (m), 125.6, 113.0 (dd, J=22.2, 3.4 Hz), 111.5 (dd, J=12.4, 3.9 Hz), 105.5 (dd, J=26.0, 26.0 Hz), 102.5 (d, J=8.3 Hz), 57.5, 53.1, 44.5, 35.7, 33.0, 29.8, 28.5. HRMS m/z [M+H]⁺ C₂₅H₂₈F₂N₃O₂⁺ calculated 440.2144, found 440.2150.

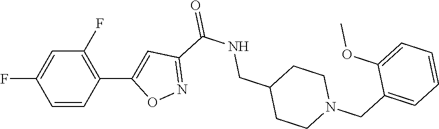 [SMILES: COc1ccccc1CN1CCC(CNC(=O)c2cc(-c3ccc(F)cc3F)on2)CC1]

### 5-(2,4-difluorophenyl)-N-((1-(2-methoxybenzyl)piperidin-4-yl)methyl)isoxazole-3-carboxamide (25)

This compound was synthesised according to the general procedure D. 5-(2,4-difluorophenyl)-N-(piperidin-4-ylmethyl)isoxazole-3-carboxamide hydrochloride (100 mg, 0.279 mmol, 1.0 eq.), TEA (39 μL, 0.28 mmol, 1.0 eq.), AcOH (16 μL, 0.28 mmol, 1.0 eq), 2-methoxybenzaldehyde (46 mg, 0.34 mmol, 1.2 eq.) and STAB-H (71 mg, 0.34 mmol, 1.2 eq.) were used. The crude product was purified by NP column chromatography (10-50% (EtOAc:MeOH:TEA, 90:5:5, v:v:v %)/cyclohexane) to obtain the title compound as an off-white solid (62 mg, 50%). LC/MS (acidic mode) Rₜ 4.18 min, purity >97%, [M+H]⁺ calculated 442.19, found 442.25. ¹H NMR (500 MHz, DMSO-d6) δ 8.88 (t, J=6.0 Hz, 1H), 8.05 (ddd, J=8.7, 8.7, 6.3 Hz, 1H), 7.57 (ddd, J=11.6, 9.2, 2.6 Hz, 1H), 7.35-7.29 (m, 1H), 7.30-7.28 (m, 1H), 7.23-7.18 (m, 1H), 7.15 (d, J=2.9 Hz, 1H), 6.95 (dd, J=8.3, 1.1 Hz, 1H), 6.90 (ddd, J=7.4, 7.4, 1.1 Hz, 1H), 3.75 (s, 3H), 3.43 (s, 2H), 3.16 (dd, J=6.4, 6.4 Hz, 2H), 2.85-2.78 (m, 2H), 1.97-1.89 (m, 2H), 1.66-1.60 (m, 2H), 1.60-1.51 (m, 1H), 1.24-1.18 (m, 2H). ¹³C NMR (126 MHz, DMSO-d6) δ 164.0 (d, J=2.2 Hz), 163.6 (dd, J=252.0, 12.7 Hz), 159.6, 159.0 (dd, J=255.2, 13.2 Hz), 158.2, 157.3, 129.6 (dd, J=10.9, 3.7 Hz), 129.6, 127.8, 126.2*, 120.1, 113.0 (dd, J=22.0, 3.5 Hz), 111.5 (dd, J=12.5, 3.9 Hz), 110.7, 105.5 (dd, J=26.0, 26.0 Hz), 102.5 (d, J=8.5 Hz), 55.7, 55.3, 53.1, 44.5, 35.5, 29.8. *Identified using HMBC HRMS m/z [M+H]⁺ C₂₄H₂₆F₂N₃O₃⁺ calculated 442.1937, found 442.1950.

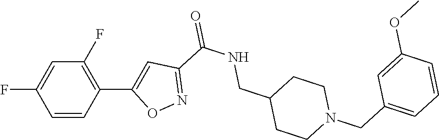 [SMILES: COc1cccc(CN2CCC(CNC(=O)c3cc(-c4ccc(F)cc4F)on3)CC2)c1]

### 5-(2,4-difluorophenyl)-N-((1-(3-methoxybenzyl)piperidin-4-yl)methyl)isoxazole-3-carboxamide (26)

This compound was synthesised according to the general procedure D. 5-(2,4-difluorophenyl)-N-(piperidin-4-ylmethyl)isoxazole-3-carboxamide hydrochloride (100 mg, 0.279 mmol, 1.0 eq.), TEA (39 μL, 0.28 mmol, 1.0 eq.), AcOH (16 μL, 0.28 mmol, 1.0 eq), 3-methoxybenzaldehyde (41 μL, 0.34 mmol, 1.2 eq.) and STAB-H (71 mg, 0.34 mmol, 1.2 eq.) were used. The crude product was purified by NP column chromatography (using a linear gradient of 10-60% (EtOAc:MeOH:TEA, 90:5:5, v:v:v %)/cyclohexane) to obtain the title compound as an off-white solid (67 mg, 54%). LC/MS (acidic mode) Rₜ 4.04 min, purity >98%, [M+H]⁺ calculated 442.19, found 442.20. ¹H NMR (500 MHz, CDCl₃) δ 7.93 (ddd, J=8.5, 8.5, 6.2 Hz, 1H), 7.22 (dd, J=8.0, 8.0 Hz, 1H), 7.09 (d, J=3.7 Hz, 1H), 7.07-7.01 (m, 1H), 7.01-6.95 (m, 1H), 6.95-6.91 (m, 1H), 6.92-6.87 (m, 1H), 6.89-6.88 (m, 1H), 6.82-6.76 (m, 1H), 3.80 (s, 3H), 3.48 (s, 2H), 3.37 (dd, J=6.5, 6.5 Hz, 2H), 2.96-2.88 (m, 2H), 2.02-1.93 (m, 2H), 1.77-1.70 (m, 2H), 1.69-1.59 (m, 1H), 1.44-1.32 (m, 2H). ¹³C NMR (126 MHz, CDCl₃) δ 164.8 (dd, J=2.7, 1.1 Hz), 164.3 (dd, J=254.8, 12.0 Hz), 159.8 (dd, J=257.4, 12.2 Hz), 159.7, 159.5, 158.9, 140.0*, 129.3, 128.9 (dd, J=10.2, 3.4 Hz), 121.6, 114.7, 112.6, 112.5 (dd, J=21.8, 3.6 Hz), 112.1 (dd, J=12.5, 3.9 Hz), 105.2 (dd, J=25.4, 25.4 Hz), 102.8 (d, J=11.4 Hz), 63.3, 55.3, 53.4, 45.1, 36.2, 30.0. *Identified using HMBC HRMS m/z [M+H]⁺ C₂₄H₂₆F₂N₃O₃⁺ calculated 442.1937, found 442.1937.

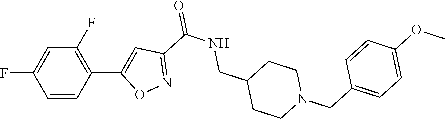 [SMILES: COc1ccc(CN2CCC(CNC(=O)c3cc(-c4ccc(F)cc4F)on3)CC2)cc1]

### 5-(2,4-difluorophenyl)-N-((1-(4-methoxybenzyl)piperidin-4-yl)methyl)isoxazole-3-carboxamide (27)

This compound was synthesised according to the general procedure D. 5-(2,4-difluorophenyl)-N-(piperidin-4-ylmethyl)isoxazole-3-carboxamide hydrochloride (100 mg, 0.279 mmol, 1.0 eq.), TEA (39 μL, 0.28 mmol, 1.0 eq.), AcOH (16 μL, 0.28 mmol, 1.0 eq), 4-methoxybenzaldehyde (41 μL, 0.34 mmol, 1.2 eq.) and STAB-H (71 mg, 0.34 mmol, 1.2 eq.) were used. Additional 4-methoxybenzaldehyde (41 μL, 0.34 mmol, 1.2 eq.) and STAB-H (71 mg, 0.34 mmol, 1.2 eq.) were added to drive the reaction to completion. The crude product was purified by NP column chromatography (using a linear gradient of 10-50% (EtOAc:MeOH:TEA, 90:5:5, v:v:v %)/cyclohexane) to obtain the title compound as an off-white solid (84 mg, 68%). LC/MS (acidic mode) Rₜ 4.08 min, purity 99%, [M+H]⁺ calculated 442.19, found 442.20. ¹H NMR (500 MHz, CDCl₃) δ 7.92 (ddd, J=8.5, 8.5, 6.2 Hz, 1H), 7.23-7.18 (m, 2H), 7.08 (d, J=3.7 Hz, 1H), 7.05-7.00 (m, 1H), 7.00-6.91 (m, 2H), 6.86-6.81 (m, 2H), 3.79 (s, 3H), 3.43 (s, 2H), 3.36 (dd, J=6.5, 6.5 Hz, 2H), 2.93-2.86 (m, 2H), 1.97-1.89 (m, 2H), 1.76-1.68 (m, 2H), 1.68-1.58 (m, 1H), 1.40-1.30 (m, 2H). ¹³C NMR (126 MHz, CDCl₃) δ 164.7 (d, J=2.7 Hz), 164.3 (dd, J=254.8, 11.9 Hz), 159.8 (dd, J=257.4, 12.2 Hz), 159.5, 158.9, 158.8, 130.5, 130.4, 128.9 (dd, J=10.1, 3.4 Hz), 113.6, 112.5 (dd, J=22.0, 3.6 Hz), 112.0 (dd, J=12.5, 3.9 Hz), 105.2 (dd, J=25.4, 25.4 Hz), 102.8 (d, J=10.6 Hz), 62.8, 55.3, 53.2, 45.1, 36.2, 30.0. HRMS m/z [M+H]⁺ C₂₄H₂₆F₂N₃O₃⁺ calculated 442.1937, found 442.1931.

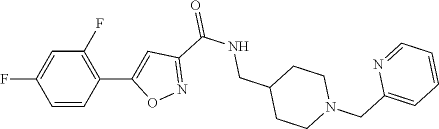 [SMILES: O=C(NCC1CCN(Cc2ccccn2)CC1)c1cc(-c2ccc(F)cc2F)on1]

### 5-(2,4-difluorophenyl)-N-((1-(pyridin-2-ylmethyl)piperidin-4-yl)methyl)isoxazole-3-carboxamide (28)

This compound was synthesised according to the general procedure D. 5-(2,4-difluorophenyl)-N-(piperidin-4-ylmethyl)isoxazole-3-carboxamide hydrochloride (100 mg, 0.279 mmol, 1.0 eq.), TEA (39 μL, 0.28 mmol, 1.0 eq.), AcOH (16 μL, 0.28 mmol, 1.0 eq), 2-pyridinecarboxaldehyde (32 μL, 0.34 mmol, 1.2 eq.) and STAB-H (71 mg, 0.34 mmol, 1.2 eq.) were used. Additional 2-pyridinecarboxaldehyde (32 μL, 0.34 mmol, 1.2 eq.) and STAB-H (71 mg, 0.34 mmol, 1.2 eq.) were added to drive the reaction to completion. The crude product was purified by NP column chromatography (using a linear gradient of 50-100% (EtOAc:MeOH:TEA, 90:5:5, v:v:v %)/cyclohexane) to obtain the title compound as an off-white solid (82 mg, 71%). LC/MS (acidic mode) Rₜ 3.82 min, purity 99%, [M+H]⁺ calculated 413.18, found 413.20. ¹H NMR (500 MHz, CDCl₃) δ 8.57-8.52 (m, 1H), 7.93 (ddd, J=8.5, 8.5, 6.2 Hz, 1H), 7.64 (ddd, J=7.7, 7.7, 1.8 Hz, 1H), 7.48-7.40 (m, 1H), 7.20-7.13 (m, 1H), 7.08 (d, J=3.7 Hz, 1H), 7.06-7.00 (m, 1H), 7.00-6.90 (m, 2H), 3.68 (s, 2H), 3.37 (dd, J=6.5, 6.5 Hz, 2H), 3.00-2.90 (m, 2H), 2.19-2.05 (m, 2H), 1.80-1.72 (m, 2H), 1.72-1.57 (m, 1H), 1.57-1.40 (m, 2H). ¹³C NMR (126 MHz, CDCl₃) δ 164.9-164.7 (m), 164.3 (dd, J=255.0, 12.0 Hz), 159.8 (dd, J=257.3, 12.0 Hz), 159.5, 158.9, 158.3*, 149.4, 136.6, 128.9 (dd, J=10.0, 3.5 Hz), 123.5, 122.2, 112.5 (dd, J=21.8, 3.6 Hz), 112.0 (dd, J=12.3, 4.1 Hz), 105.2 (dd, J=25.4, 25.4 Hz), 102.8 (d, J=10.6 Hz), 64.8, 53.6, 45.0, 36.0, 29.8. *Identified using HMBC HRMS m/z [M+H]⁺ C₂₂H₂₃F₂N₄O₂⁺ calculated 413.1784, found 413.1785.

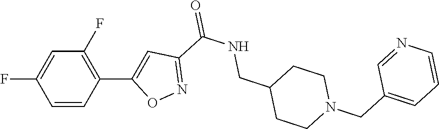 [SMILES: O=C(NCC1CCN(Cc2cccnc2)CC1)c1cc(-c2ccc(F)cc2F)on1]

### 5-(2,4-difluorophenyl)-N-((1-(pyridin-3-ylmethyl)piperidin-4-yl)methyl)isoxazole-3-carboxamide (29)

This compound was synthesised according to the general procedure D. 5-(2,4-difluorophenyl)-N-(piperidin-4-ylmethyl)isoxazole-3-carboxamide hydrochloride (100 mg, 0.279 mmol, 1.0 eq.), TEA (39 μL, 0.28 mmol, 1.0 eq.), AcOH (16 μL, 0.28 mmol, 1.0 eq), 3-pyridinecarboxaldehyde (31 μL, 0.34 mmol, 1.2 eq.) and STAB-H (71 mg, 0.34 mmol, 1.2 eq.) were used. Additional 3-pyridinecarboxaldehyde (31 μL, 0.34 mmol, 1.2 eq.) and STAB-H (71 mg, 0.34 mmol, 1.2 eq.) were added to drive the reaction to completion. The crude product was purified by NP column chromatography (using a linear gradient of 50-100% (EtOAc:MeOH:TEA, 90:5:5, v:v:v %)/cyclohexane) to obtain the title compound as an off-white solid (83 mg, 72%). LC/MS (acidic mode) Rₜ 3.55 min, purity >95%, [M+H]⁺ calculated 413.18, found 413.15. ¹H NMR (500 MHz, DMSO-d6) δ 8.88 (t, J=6.0 Hz, 1H), 8.48-8.47 (m, 1H), 8.46-8.44 (m, 1H), 8.05 (ddd, J=8.7, 8.7, 6.3 Hz, 1H), 7.72-7.66 (m, 1H), 7.58 (ddd, J=11.6, 9.3, 2.6 Hz, 1H), 7.37-7.29 (m, 2H), 7.15 (d, J=3.0 Hz, 1H), 3.48 (s, 2H), 3.16 (dd, J=6.4, 6.4 Hz, 2H), 2.82-2.72 (m, 2H), 1.98-1.88 (m, 2H), 1.74-1.60 (m, 2H), 1.67-1.47 (m, 1H), 1.24-1.12 (m, 2H). ¹³C NMR (126 MHz, DMSO-d6) δ 164.3-163.8 (m), 163.6 (dd, J=252.0, 12.3 Hz), 159.6, 159.0 (dd, J=255.2, 12.7 Hz), 158.2, 150.0, 148.2, 136.5, 133.9*, 129.6 (dd, J=10.4, 3.2 Hz), 123.4, 113.0 (dd, J=22.1, 3.4 Hz), 111.5 (dd, J=12.6, 3.8 Hz), 105.5 (dd, J=25.9, 25.9 Hz), 102.5 (d, J=8.4 Hz), 59.4, 52.8, 44.4, 35.4, 29.6. *Identified using HMBC HRMS m/z [M+H]⁺ C₂₂H₂₃F₂N₄O₂⁺ calculated 413.1784, found 413.1793.

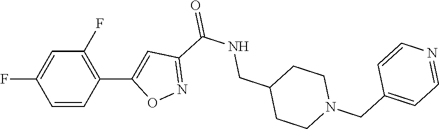 [SMILES: O=C(NCC1CCN(Cc2ccncc2)CC1)c1cc(-c2ccc(F)cc2F)on1]

### 5-(2,4-difluorophenyl)-N-((1-(pyridin-4-ylmethyl)piperidin-4-yl)methyl)isoxazole-3-carboxamide (30)

This compound was synthesised according to the general procedure D. 5-(2,4-difluorophenyl)-N-(piperidin-4-ylmethyl)isoxazole-3-carboxamide hydrochloride (100 mg, 0.279 mmol, 1.0 eq.), TEA (39 μL, 0.28 mmol, 1.0 eq.), AcOH (16 μL, 0.28 mmol, 1.0 eq), 4-pyridinecarboxaldehyde (32 μL, 0.34 mmol, 1.2 eq.) and STAB-H (71 mg, 0.34 mmol, 1.2 eq.) were used. Additional 4-pyridinecarboxaldehyde (32 μL, 0.34 mmol, 1.2 eq.) and STAB-H (71 mg, 0.34 mmol, 1.2 eq.) were added to drive the reaction to completion. The crude product was purified by NP column chromatography (using a linear gradient of 50-100% (EtOAc:MeOH:TEA, 90:5:5, v:v:v %)/cyclohexane) to obtain the title compound as an off-white solid (86 mg, 75%). LC/MS (acidic mode) Rₜ 3.45 min. purity >99%, [M+H]⁺ calculated 413.18, found 413.15. ¹H NMR (500 MHz, DMSO-d6) δ 8.89 (t, J=6.0 Hz, 1H), 8.49 (d, J=5.9 Hz, 2H), 8.05 (ddd, J=8.7, 8.6, 6.3 Hz, 1H), 7.57 (ddd, J=11.5, 9.2, 2.6 Hz, 1H), 7.35-7.28 (m, 3H), 7.15 (d, J=3.0 Hz, 1H), 3.48 (s, 2H), 3.17 (dd, J=6.4, 6.4 Hz, 2H), 2.84-2.70 (m, 2H), 2.02-1.86 (m, 2H), 1.70-1.61 (m, 2H), 1.61-1.50 (m, 1H), 1.27-1.13 (m, 2H). ¹³C NMR (126 MHz, DMSO-d6) δ 164.4-163.8 (m), 163.6 (dd, J=252.3, 12.5 Hz), 159.6, 159.0 (dd, J=255.2, 13.2 Hz), 158.2, 149.5, 148.9*, 129.6 (dd, J=10.3, 3.3 Hz), 123.7, 113.7-111.5 (m), 111.4 (d, J=3.6 Hz), 105.5 (dd, J=26.0, 26.0 Hz), 102.5 (d, J=8.5 Hz), 60.9, 52.9, 44.4, 35.4, 29.6. *Identified using HMBC HRMS m/z [M+H]⁺ C₂₂H₂₃F₂N₄O₂⁺ calculated 413.1784, found 413.1791.

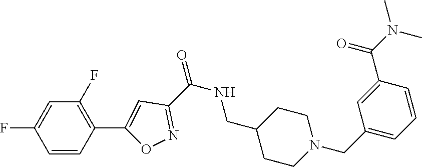 [SMILES: CN(C)C(=O)c1cccc(CN2CCC(CNC(=O)c3cc(-c4ccc(F)cc4F)on3)CC2)c1]

### 5-(2,4-difluorophenyl)-N-((1-(3-(dimethylcarbamoyl)benzyl)piperidin-4-yl)methyl)isoxazole-3-carboxamide(31)

This compound was synthesised according to the general procedure D. 5-(2,4-difluorophenyl)-N-(piperidin-4-ylmethyl)isoxazole-3-carboxamide hydrochloride (100 mg, 0.279 mmol, 1.0 eq.), TEA (39 μL, 0.28 mmol, 1.0 eq.), 3-formyl-N,N-dimethylbenzamide (59 mg, 0.34 mmol, 1.2 eq.) and STAB-H (71 mg, 0.34 mmol, 1.2 eq.) were used. Additional 3-formyl-N,N-dimethylbenzamide (59 mg, 0.34 mmol, 1.2 eq.) and STAB-H (142 mg, 0.671 mmol, 2.4 eq.) were added to drive the reaction to completion. The crude product was purified by NP column chromatography (using a linear gradient of 10-60% (EtOAc:MeOH:TEA, 90:5:5, v:v:v %)/cyclohexane) to obtain the title compound as an off-white solid (32 mg, 24%). LC/MS (acidic mode) Rₜ 3.78 min, purity 98%, [M+H]⁺ calculated 483.22, found 483.15. ¹H NMR (500 MHz, DMSO-d6) δ 8.98-8.84 (m, 1H), 8.12-8.00 (m, 1H), 7.61-7.53 (m, 1H), 7.49-7.18 (m, 4H), 7.27-7.19 (m, 1H), 7.20-7.11 (m, 1H), 3.47 (s, 2H), 3.22-3.11 (m, 2H), 2.97 (s, 3H), 2.88 (s, 3H), 2.82-2.75 (m, 2H), 1.94-1.85 (m, 2H), 1.77-1.50 (m, 3H), 1.26-1.12 (m, 2H). ¹³C NMR (126 MHz, DMSO-d6) δ 170.2, 164.0, 163.6 (dd, J=252.0, 12.3 Hz), 159.6, 159.0 (dd, J=255.2, 12.7 Hz), 158.2, 138.9, 136.3, 129.7 (d, J=3.1 Hz), 129.6, 128.1, 127.0, 125.4, 113.0 (dd, J=22.2, 3.4 Hz), 111.5 (dd, J=12.5, 3.9 Hz), 105.5 (dd, J=26.0, 26.0 Hz), 102.5 (d, J=8.2 Hz), 61.9, 52.8, 44.4, 35.5, 34.7, 29.7. Rotamers are observed. HRMS m/z [M+H]⁺ C₂₆H₂₉F₂N₄O₃⁺ calculated 483.2202, found 483.2220.

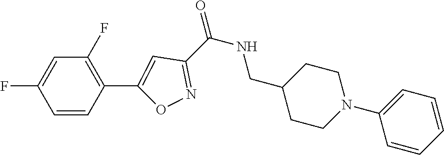 [SMILES: O=C(NCC1CCN(c2ccccc2)CC1)c1cc(-c2ccc(F)cc2F)on1]

### 5-(2,4-difluorophenyl)-N-((1-phenylpiperidin-4-yl)methyl)isoxazole-3-carboxamide (32)

A suspension of 5-(2,4-difluorophenyl)-N-(piperidin-4-ylmethyl)isoxazole-3-carboxamide hydrochloride (98 mg, 0.274 mmol, 1.0 eq.), bromobenzene (58 μL, 0.55 mmol, 2.0 eq.), NaOtBu (53 mg, 0.55 mmol, 2.0 eq.)) and S-Phos (11 mg, 0.027 mmol, 0.1 eq.) in dry PhMe (1.4 mL). Pd₂dba₃·CHCl₃ (14 mg, 0.14 mmol, 0.05 eq.) was added and the reaction mixture was heated to reflux by microwave irradiation for 19 h. Additional S-Phos (11 mg, 0.027 mmol, 0.1 eq.) and Pd₂dba₃·CHCl₃ (14 mg, 0.14 mmol, 0.05 eq.) were added and the reaction mixture was heated for an additional 14 h. The reaction mixture was cooled, filtered over celite and the residue washed with EtOAc. The filtrate was concentrated in vacuo. The resulting crude was purified by NP column chromatography (using a linear gradient of 10-50% (EtOAc:MeOH:TEA, 90:5:5, v:v:v %)/cyclohexane) to obtain a yellow solid. The solid was triturated with MeOH to obtain the title compound as a white solid (15 mg, 14%). LC/MS (acidic mode) Rₜ 4.40 min, purity 99%, [M+H]⁺ calculated 398.17, found 398.10. ¹H NMR (500 MHz, DMSO-d6) δ 8.95 (t, J=6.0 Hz, 1H), 8.06 (ddd, J=8.6, 8.6, 6.3 Hz, 1H), 7.58 (ddd, J=11.6, 9.3, 2.5 Hz, 1H), 7.33 (ddd, J=8.5, 8.5, 2.6 Hz, 1H), 7.21-7.15 (m, 3H), 6.92 (d, J=8.1 Hz, 2H), 6.73 (t, J=7.2 Hz, 1H), 3.72-3.64 (m, 2H), 3.25-3.18 (m, 2H), 2.67-2.58 (m, 2H), 1.81-1.69 (m, 3H), 1.33-1.23 (m, 2H). ¹³C NMR (126 MHz, DMSO-d6) δ 164.0 (d, J=1.7 Hz), 163.6 (dd, J=251.6, 12.2 Hz), 159.6, 159.0 (dd, J=255.3, 12.7 Hz), 158.3, 151.3, 129.6 (dd, J=10.1, 3.3 Hz), 128.9, 118.4, 115.8, 113.0 (dd, J=22.2, 3.5 Hz), 111.5 (dd, J=12.6, 4.0 Hz), 105.5 (dd, J=26.0, 26.0 Hz), 102.5 (d, J=9.1 Hz), 48.5, 44.3, 35.5, 29.3. HRMS m/z [M+H]⁺ C₂₂H₂₂F₂N₃O₂⁺ calculated 398.1675, found 398.1680.

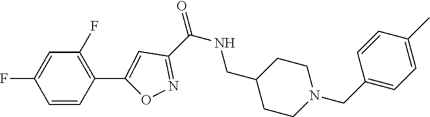 [SMILES: Cc1ccc(CN2CCC(CNC(=O)c3cc(-c4ccc(F)cc4F)on3)CC2)cc1]

### 5-(2,4-difluorophenyl)-N-((1-(4-methylbenzyl)piperidin-4-yl)methyl)isoxazole-3-carboxamide (33)

This compound was synthesised according to general procedure C. 5-(2,4-difluorophenyl)-N-(piperidin-4-ylmethyl)isoxazole-3-carboxamide hydrochloride (100 mg, 0.279 mmol, 1.0 eq.), K₂CO₃ (77 mg, 0.56 mmol, 2.0 eq.) and 4-methylbenzyl bromide (57 mg, 0.31 mmol, 1.1 eq.) were used. The crude product was purified by NP column chromatography (using a linear gradient of 10-50% (EtOAc:MeOH:TEA, 90:5:5, v:v:v %)/cyclohexane) to obtain the title compound as a white solid (28 mg, 24%). LC/MS (acidic mode) Rₜ 4.50 min, purity 99%, [M+H]⁺ calculated 426.20, found 426.20. ¹H NMR (500 MHz, DMSO-d6) δ 8.89 (t, J=6.0 Hz, 1H), 8.05 (ddd, J=8.7, 8.7, 6.3 Hz, 1H), 7.57 (ddd, J=11.5, 9.2, 2.6 Hz, 1H), 7.32 (ddd, J=8.5, 8.5, 2.6 Hz, 1H), 7.23-7.08 (m, 5H), 3.48-3.41 (m, 2H), 3.16 (dd, J=6.3, 6.3 Hz, 2H), 2.86-2.74 (m, 2H), 2.27 (s, 3H), 2.02-1.84 (m, 2H), 1.69-1.61 (m, 2H), 1.61-1.52 (m, 1H), 1.26-1.12 (m, 2H). ¹³C NMR (126 MHz, DMSO-d6) δ 164.0, 164.0, 163.7 (dd, J=251.8, 12.0 Hz), 159.6, 159.0 (dd, J=255.2, 12.7 Hz), 158.2, 136.5*130.6-129.4 (m), 128.9, 128.8, 113.0 (dd, J=22.1, 3.4 Hz), 111.5 (dd, J=12.5, 3.9 Hz), 105.5 (dd, J=26.0, 26.0 Hz), 102.5 (d, J=8.4 Hz), 61.9, 52.7, 44.4, 35.4, 29.5, 20.7. *Identified using HMBC HRMS m/z [M+H]⁺ C₂₄H₂₆F₂N₃O₂⁺ calculated 426.1988, found 426.1994.

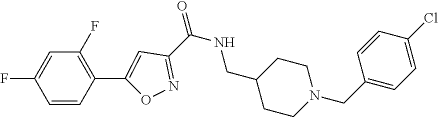 [SMILES: O=C(NCC1CCN(Cc2ccc(Cl)cc2)CC1)c1cc(-c2ccc(F)cc2F)on1]

### N-((1-(4-chlorobenzyl)piperidin-4-yl)methyl)-5-(2,4-difluorophenyl)isoxazole-3-carboxamide (34)

This compound was synthesised according to general procedure C. 5-(2,4-difluorophenyl)-N-(piperidin-4-ylmethyl)isoxazole-3-carboxamide hydrochloride (100 mg, 0.279 mmol, 1.0 eq.), K₂CO₃ (77 mg, 0.56 mmol, 2.0 eq.) and 4-chlorobenzyl bromide (63 mg, 0.31 mmol, 1.1 eq.) were used. The crude product was purified by NP column chromatography (using a linear gradient of 10-50% (EtOAc:MeOH:TEA, 90:5:5, v:v:v %)/cyclohexane) to obtain the title compound as a white solid (71 mg, 57%). LC/MS (acidic mode) Rₜ 4.52 min, purity 98%, [M+H]⁺ calculated 446.14, found 446.10. ¹H NMR (500 MHz, DMSO-d6) δ 8.88 (t, J=6.0 Hz, 1H), 8.05 (ddd, J=8.7, 8.7, 6.3 Hz, 1H), 7.57 (ddd, J=11.6, 9.2, 2.6 Hz, 1H), 7.37-7.28 (m, 5H), 7.14 (d, J=3.0 Hz, 1H), 3.42 (s, 2H), 3.15 (dd, J=6.4, 6.4 Hz, 2H), 2.81-2.71 (m, 2H), 1.93-1.85 (m, 2H), 1.67-1.60 (m, 2H), 1.60-1.51 (m, 1H), 1.23-1.13 (m, 2H). ¹³C NMR (126 MHz, DMSO-d6) δ 164.0, 163.6 (dd, J=251.5, 12.2 Hz), 159.6, 159.0 (dd, J=255.4, 12.9 Hz), 158.2, 137.8 (d, J=9.7 Hz), 131.3, 130.5, 129.6 (dd, J=10.3, 3.2 Hz), 128.1, 113.0 (dd, J=22.2, 3.5 Hz), 111.5 (dd, J=12.7, 4.1 Hz), 105.5 (dd, J=26.0, 26.0 Hz), 102.5 (d, J=8.5 Hz), 61.4, 52.8, 44.4, 35.5, 29.7. HRMS m/z [M+H]⁺ C₂₃H₂₃ClF₂N₃O₂⁺ calculated 446.1441, found 446.1461.

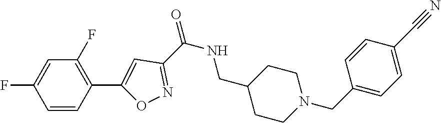 [SMILES: N#Cc1ccc(CN2CCC(CNC(=O)c3cc(-c4ccc(F)cc4F)on3)CC2)cc1]

### N-((1-(4-cyanobenzyl)piperidin-4-yl)methyl)-5-(2,4-difluorophenyl)isoxazole-3-carboxamide (35)

This compound was synthesised according to general procedure C. 5-(2,4-difluorophenyl)-N-(piperidin-4-ylmethyl)isoxazole-3-carboxamide hydrochloride (100 mg, 0.279 mmol, 1.0 eq.), K₂CO₃ (77 mg, 0.56 mmol, 2.0 eq.) and 4-cyanobenzyl bromide (60 mg, 0.31 mmol, 1.1 eq.) were used. The crude product was purified by NP column chromatography (using a linear gradient of 10-50% (EtOAc:MeOH:TEA, 90:5:5, v:v:v %)/cyclohexane) to obtain the title compound as a white solid (66 mg, 54%). LC/MS (acidic mode) Rₜ 4.05 min, purity >97%, [M+H]⁺ calculated 437.18, found 437.15. ¹H NMR (500 MHz, DMSO-d6) δ 8.88 (t, J=6.0 Hz, 1H), 8.09-8.01 (m, 1H), 7.77 (d, J=7.8 Hz, 2H), 7.61-7.53 (m, 1H), 7.49 (d, J=7.9 Hz, 2H), 7.36-7.29 (m, 1H), 7.14 (d, J=2.9 Hz, 1H), 3.52 (s, 2H), 3.16 (dd, J=6.4, 6.4 Hz, 2H), 2.80-2.71 (m, 2H), 1.97-1.88 (m, 2H), 1.68-1.61 (m, 2H), 1.61-1.53 (m, 1H), 1.24-1.15 (m, 2H). ¹³C NMR (126 MHz, DMSO-d6) δ 164.0, 163.6 (dd, J=252.0, 12.7 Hz), 159.6, 159.0 (dd, J=255.2, 12.7 Hz), 158.2, 145.0, 132.1, 129.6 (dd, J=10.4, 3.4 Hz), 129.4, 119.0, 113.0 (d, J=22.1 Hz), 111.5 (dd, J=12.5, 3.9 Hz), 109.6, 105.5 (dd, J=26.0, 26.0 Hz), 102.5 (d, J=8.6 Hz), 61.7, 52.9, 44.4, 35.4, 29.7. HRMS m/z [M+H]⁺ C₂₄H₂₃F₂N₄O₂⁺ calculated 437.1784, found 437.1792.

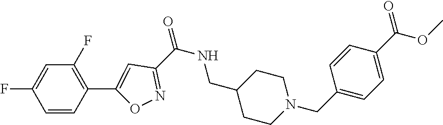 [SMILES: COC(=O)c1ccc(CN2CCC(CNC(=O)c3cc(-c4ccc(F)cc4F)on3)CC2)cc1]

### Methyl 4-((4-((5-(2,4-difluorophenyl)isoxazole-3-carboxamido)methyl)piperidin-1-yl) methyl) benzoate (36)

This compound was synthesised according to general procedure C. 5-(2,4-difluorophenyl)-N-(piperidin-4-ylmethyl)isoxazole-3-carboxamide hydrochloride (100 mg, 0.279 mmol, 1.0 eq.), K₂CO₃ (77 mg, 0.56 mmol, 2.0 eq.) and methyl 4-(bromomethyl)benzoate (70 mg, 0.31 mmol, 1.1 eq.) were used. The crude product was purified by NP column chromatography (using a linear gradient of 10-50% (EtOAc:MeOH:TEA, 90:5:5, v:v:v %)/cyclohexane) to obtain the title compound as a white solid (92 mg, 70%). LC/MS (acidic mode) Rₜ 4.15 min, purity 98%, [M+H]⁺ calculated 470.19, found 470.15. ¹H NMR (500 MHz, DMSO-d6) δ 8.88 (t, J=6.0 Hz, 1H), 8.04 (ddd, J=8.7, 8.7, 6.4 Hz, 1H), 7.93-7.88 (m, 2H), 7.57 (ddd, J=11.6, 9.2, 2.6 Hz, 1H), 7.46-7.40 (m, 2H), 7.36-7.29 (m, 1H), 7.14 (d, J=2.9 Hz, 1H), 3.83 (s, 3H), 3.51 (s, 2H), 3.16 (dd, J=6.4 Hz, 6.4 Hz, 2H), 2.81-2.72 (m, 2H), 1.96-1.87 (m, 2H), 1.67-1.61 (m, 2H), 1.61-1.52 (m, 1H), 1.25-1.14 (m, 2H). ¹³C NMR (126 MHz, DMSO-d6) δ 166.2, 164.0 (d, J=2.7 Hz), 163.6 (dd, J=251.8, 12.5 Hz), 159.6, 159.0 (dd, J=255.2, 12.7 Hz), 158.2, 144.7, 129.6 (dd, J=10.3, 3.4 Hz), 129.1, 128.8, 128.2, 113.0 (dd, J=22.1, 3.5 Hz), 111.5 (dd, J=12.3, 3.6 Hz), 105.5 (dd, J=25.9, 25.9 Hz), 102.5 (d, J=8.5 Hz), 61.9, 53.0, 52.0, 44.4, 35.5, 29.7. HRMS m/z [M+H]⁺ C₂₅H₂₆F₂N₃O₄⁺ calculated 470.1886, found 470.1900.

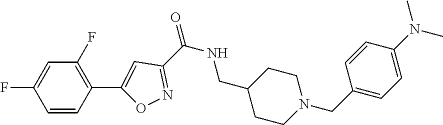 [SMILES: CN(C)c1ccc(CN2CCC(CNC(=O)c3cc(-c4ccc(F)cc4F)on3)CC2)cc1]

### 5-(2,4-difluorophenyl)-N-((1-(4-(dimethylamino)benzyl)piperidin-4-yl)methyl)isoxazole-3-carboxamide (37)

This compound was synthesised according to general procedure D. 5-(2,4-difluorophenyl)-N-(piperidin-4-ylmethyl)isoxazole-3-carboxamide hydrochloride (100 mg, 0.279 mmol, 1.0 eq.), TEA (39 μL, 0.28 mmol, 1.0 eq.), 4-(dimethylamino)benzaldehyde (50 mg, 0.34 mmol, 1.2 eq.) and STAB-H (148 mg, 0.699 mmol, 2.5 eq.) were used. Additional 4-(dimethylamino)benzaldehyde (25 mg, 0.17 mmol, 0.6 eq.) and STAB-H (36 mg, 0.17 mmol, 0.6 eq.) were added to drive the reaction to completion. The crude product was purified by NP column chromatography (using a linear gradient of 30-70% (EtOAc:MeOH:TEA, 90:5:5, v:v:v %)/cyclohexane) to obtain the title compound as a white solid (73 mg, 58%). LC/MS (acidic mode) Rₜ 4.27 min, purity 98%, [M+H]⁺ calculated 455.23, found 455.20. ¹H NMR (500 MHz, DMSO-d6) δ 8.87 (t, J=5.9 Hz, 1H), 8.05 (ddd, J=8.7, 8.7, 6.4 Hz, 1H), 7.58 (ddd, J=11.5, 9.2, 2.5 Hz, 1H), 7.32 (ddd, J=8.6, 8.3, 2.1 Hz, 1H), 7.15 (d, J=3.0 Hz, 1H), 7.07 (dd, J=9.3, 2.6 Hz, 2H), 6.67-6.64 (m, 2H), 3.32-3.29 (m, 2H), 3.15 (dd, J=6.4, 6.4 Hz, 2H), 2.85 (s, 6H), 2.80-2.73 (m, 2H), 1.86-1.77 (m, 2H), 1.65-1.58 (m, 2H), 1.58-1.49 (m, 1H), 1.20-1.09 (m, 2H). ¹³C NMR (126 MHz, DMSO-d6) δ 164.0, 163.6 (dd, J=251.6, 12.4 Hz), 159.6, 159.0 (dd, J=255.1, 12.7 Hz), 158.2, 149.5, 129.6, 129.2, 125.9, 113.0 (dd, J=22.1, 3.5 Hz), 112.2, 112.8-110.3 (m), 105.5 (dd, J=26.0, 26.0 Hz), 102.5 (d, J=8.2 Hz), 62.1, 52.7, 44.5, 40.3, 35.6, 29.7. HRMS m/z [M+H-p-(NMe₂)Bn]⁺ C₁₆H₁₈F₂N₃O₂⁺ calculated 322.1362, found 322.1382.

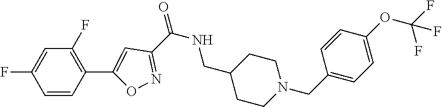 [SMILES: O=C(NCC1CCN(Cc2ccc(OC(F)(F)F)cc2)CC1)c1cc(-c2ccc(F)cc2F)on1]

### 5-(2,4-difluorophenyl-N-((1-(4-(trifluoromethoxy)benzyl)piperidin-4-yl)methyl)isoxazole-3-carboxamide(38)

This compound was synthesised according to general procedure D. 5-(2,4-difluorophenyl)-N-(piperidin-4-ylmethyl)isoxazole-3-carboxamide hydrochloride (100 mg, 0.279 mmol, 1.0 eq.), TEA (39 μL, 0.28 mmol, 1.0 eq.), 4-(trifluoromethoxy)benzaldehyde (64 mg, 0.34 mmol, 1.2 eq.) and STAB-H (148 mg, 0.699 mmol, 2.5 eq.) were used. For this compound no column chromatography was necessary. The crude product was purified by trituration with MeOH to obtain the title compound as a white solid (66 mg, 48%). LC/MS (acidic mode) Rₜ 4.60 min, purity 98%, [M+H]⁺ calculated 496.17, found 496.20. ¹H NMR (500 MHz, CDCl₃) δ 7.94 (ddd, J=8.5, 8.5, 6.1 Hz, 1H), 7.40-7.34 (m, 2H), 7.18-7.14 (m, 2H), 7.09 (d, J=3.8 Hz, 1H), 7.07-7.01 (m, 1H), 7.01-6.95 (m, 1H), 6.95-6.89 (m, 1H), 3.54 (s, 2H), 3.38 (dd, J=6.5, 6.5 Hz, 2H), 3.00-2.87 (m, 2H), 2.04 (s, 2H), 1.82-1.73 (m, 2H), 1.73-1.62 (m, 1H), 1.50-1.38 (m, 2H). ¹³C NMR (126 MHz, CDCl₃) δ 164.9, 164.3 (dd, J=255.5, 12.3 Hz), 159.5, 159.0, 130.7, 128.9 (dd, J=10.0, 3.6 Hz), 120.9, 112.6 (dd, J=22.0, 3.4 Hz), 112.1 (dd, J=12.8, 4.2 Hz), 105.2 (dd, J=25.2, 25.2 Hz), 102.7, 62.3, 53.2, 45.0, 35.9, 29.7. Four quaternary carbon signals are missing in this spectrum. HRMS m/z [M+H]⁺ C₂₄H₂₃F₅N₃O₃⁺ calculated 496.1654, found 496.1678.

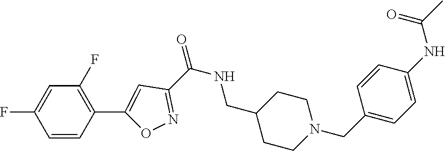 [SMILES: CC(=O)Nc1ccc(CN2CCC(CNC(=O)c3cc(-c4ccc(F)cc4F)on3)CC2)cc1]

### N-((1-(4-acetamidobenzyl)piperidin-4-yl)methyl)-5-(2,4-difluorophenyl)isoxazole-3-carboxamide (39)

This compound was synthesised according to general procedure D. 5-(2,4-difluorophenyl)-N-(piperidin-4-ylmethyl)isoxazole-3-carboxamide hydrochloride (100 mg, 0.279 mmol, 1.0 eq.), TEA (39 μL, 0.28 mmol, 1.0 eq.), 4-acetamidobenzaldehyde (55 mg, 0.34 mmol, 1.2 eq.) and STAB-H (148 mg, 0.699 mmol, 2.5 eq.) were used. Additional 4-acetamidobenzaldehyde (55 mg, 0.34 mmol, 1.2 eq.) and STAB-H (148 mg, 0.699 mmol, 2.5 eq.) were added to drive the reaction to completion. The crude product was purified by trituration in MeOH to obtain the title compound as a white solid (39 mg, 30%). LC/MS (acidic mode) Rₜ 3.53 min, purity 98%, [M+H]⁺ calculated 469.20, found 469.25. ¹H NMR (500 MHz, DMSO-d6) δ 9.88 (s, 1H), 8.87 (t, J=5.9 Hz, 1H), 8.09-8.00 (m, 1H), 7.62-7.54 (m, 1H), 7.50 (d, J=8.0 Hz, 2H), 7.37-7.28 (m, 1H), 7.21-7.12 (m, 3H), 3.35 (s, 2H), 3.15 (dd, J=6.4, 6.4 Hz, 2H), 2.81-2.72 (m, 2H), 2.02 (s, 3H), 1.90-1.80 (m, 2H), 1.67-1.59 (m, 2H), 1.59-1.50 (m, 1H), 1.23-1.11 (m, 2H). ¹³C NMR (126 MHz, DMSO-d6) δ 168.1, 164.0, 163.6 (dd, J=251.9, 12.5 Hz), 159.6, 159.0 (dd, J=255.2, 12.8 Hz), 158.2, 138.0, 133.1, 129.6 (dd, J=10.3, 3.4 Hz), 129.0, 118.7, 113.0 (dd, J=22.1, 3.3 Hz), 111.5 (dd, J=12.3, 3.8 Hz), 105.5 (dd, J=25.9, 25.9 Hz), 102.5 (d, J=8.5 Hz), 62.0, 52.8, 44.4, 35.6, 29.7, 23.9. HRMS m/z [M+H]⁺ C₂₅H₂₇F₂N₄O₃⁺ calculated 469.2046, found 469.2066.

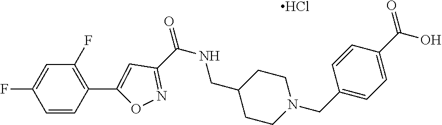 [SMILES: O=C(O)c1ccc(CN2CCC(CNC(=O)c3cc(-c4ccc(F)cc4F)on3)CC2)cc1 ; Cl]

### 4-((4-((5-(2,4-difluorophenyl)isoxazole-3-carboxamido)methyl)piperidin-1-yl)methyl)benzoic acid hydrochloride (40)

5-(2,4-difluorophenyl)-N-(piperidin-4-ylmethyl)isoxazole-3-carboxamide hydrochloride (100 mg, 0.279 mmol, 1.0 eq.) was suspended in THE (2.8 mL) that contained TEA (39 μL, 0.28 mmol, 1.0 eq.) and 4-formylbenzaldehyde (50 mg, 0.34 mmol, 1.2 eq.). The mixture was stirred at RT for 10 min. Subsequently, STAB-H (148 mg, 0.699 mmol, 2.5 eq.) was added and the reaction mixture was stirred overnight at RT. Additional 4-formylbenzoic acid (55 mg, 0.34 mmol, 1.2 eq.) and STAB-H (148 mg, 0.699 mmol, 2.5 eq.) were added to drive the reaction to completion. MeOH was added to terminate the reaction and the volatiles were removed by in vacuo. The resulting solids were mixed with an aqueous solution of NaOH (2M) and extracted with DCM (3 times). The combined organic layers were dried over anhydrous Na₂SO₄, filtered and concentrated in vacuo. The resulting residue was purified using RP column chromatography (using a linear gradient of 10-70% ACN/H₂O with 0.1% FA). The product fractions were combined and an excess of an aqueous solution of HCl (3M) was added. The resulting mixture was lyophilised to obtain the title compound as a white solid (20 mg, 16%). LC/MS (acidic mode) Rₜ 3.16 min, purity 97%, [M+H]⁺ calculated 456.17, found 456.20. ¹H NMR (500 MHz, DMSO-d6) δ 13.15 (s, 1H), 10.79-10.49 (m, 1H), 9.09-8.99 (m, 1H), 8.08-8.02 (m, 1H), 7.99 (d, J=8.0 Hz, 2H), 7.72 (d, J=8.0 Hz, 2H), 7.58 (ddd, J=11.4, 9.1, 2.5 Hz, 1H), 7.33 (ddd, J=8.5, 8.5, 2.5 Hz, 1H), 7.16 (d, J=3.0 Hz, 1H), 4.33 (d, J=4.6 Hz, 2H), 3.33-3.29 (m, 2H), 3.17 (dd, J=6.3, 6.3 Hz, 2H), 2.96-2.85 (m, 2H), 1.88-1.77 (m, 3H), 1.60-1.48 (m, 2H). ¹³C NMR (126 MHz, DMSO-d6) δ 166.9, 164.1 (d, J=2.5 Hz), 163.7 (dd, J=252.3, 12.4 Hz), 159.5, 159.0 (dd, J=255.2, 12.8 Hz), 158.4, 134.5, 131.7, 129.9-129.4 (m), 129.5, 113.0 (dd, J=22.2, 3.4 Hz), 111.5 (dd, J=12.4, 3.8 Hz), 105.5 (dd, J=25.9, 25.9 Hz), 102.5 (d, J=8.5 Hz), 58.5, 51.3, 43.6, 33.5, 26.6. One carbon signal could not be detected even after 2D NMR analysis. HRMS m/z [M+H]⁺ C₂₄H₂₄F₂N₃O₄⁺ calculated 456.1729, found 456.1735.

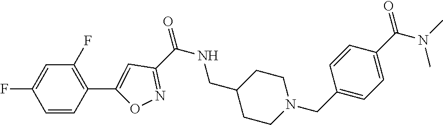 [SMILES: CN(C)C(=O)c1ccc(CN2CCC(CNC(=O)c3cc(-c4ccc(F)cc4F)on3)CC2)cc1]

### 5-(2,4-difluorophenyl)-N-((1-(4-(dimethylcarbamoyl)benzyl)piperidin-4-yl)methyl)isoxazole-3-carboxamide (41)

This compound was synthesised according to the general procedure D. 5-(2,4-difluorophenyl)-N-(piperidin-4-ylmethyl)isoxazole-3-carboxamide hydrochloride (100 mg, 0.279 mmol, 1.0 eq.), TEA (39 μL, 0.28 mmol, 1.0 eq.), 4-formyl-N,N-dimethylbenzamide (59 mg, 0.34 mmol, 1.2 eq.) and STAB-H (71 mg, 0.34 mmol, 1.2 eq.) were used. Additional 4-formyl-N,N-dimethylbenzamide (59 mg, 0.34 mmol, 1.2 eq.) and STAB-H (142 mg, 0.671 mmol, 2.4 eq.) were added to drive the reaction to completion. The crude product was purified by NP column chromatography (using a linear gradient of 10-45% (EtOAc:MeOH:TEA, 90:5:5, v:v:v %)/cyclohexane) to obtain the title compound as an off-white solid (28 mg, 21%). LC/MS (acidic mode) Rₜ 3.13 min, purity 95%, [M+H]⁺ calculated 483.22, found 483.30. ¹H NMR (500 MHz, DMSO-d6) δ 8.89 (t, J=5.9 Hz, 1H), 8.10-8.00 (m, 1H), 7.61-7.54 (m, 1H), 7.39-7.29 (m, 5H), 7.15 (d, J=2.9 Hz, 1H), 3.46 (s, 2H), 3.16 (dd, J=6.4, 6.4 Hz, 2H), 2.96 (s, 3H), 2.90 (s, 3H), 2.79 (d, J=11.1 Hz, 2H), 1.95-1.85 (m, 2H), 1.69-1.60 (m, 2H), 1.60-1.52 (m, 1H), 1.25-1.14 (m, 2H). ¹³C NMR (126 MHz, DMSO-d6) δ 170.1, 164.0, 163.6 (dd, J=251.8, 12.5 Hz), 159.6, 159.0 (dd, J=255.2, 12.4 Hz), 158.2, 140.1, 134.9, 129.6 (dd, J=10.3, 3.3 Hz), 128.4, 126.9, 113.0 (dd, J=21.9, 3.5 Hz), 112.3-110.7 (m), 105.5 (dd, J=25.9, 25.9 Hz), 102.5 (d, J=8.7 Hz), 62.0, 52.9, 44.4, 38.5, 35.5, 29.7. Rotamers are observed. HRMS m/z [M+H]⁺ C₂₆H₂₉F₂N₄O₃⁺ calculated 483.2202, found 483.2221.

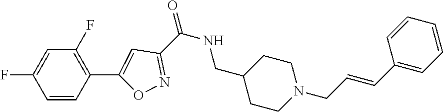 [SMILES: O=C(NCC1CCN(C/C=C/c2ccccc2)CC1)c1cc(-c2ccc(F)cc2F)on1]

### N-((1-cinnamylpiperidin-4-yl)methyl)-5-(2,4-difluorophenyl)isoxazole-3-carboxamide (42)

5-(2,4-difluorophenyl)-N-(piperidin-4-ylmethyl)isoxazole-3-carboxamide hydrochloride (100 mg, 0.279 mmol, 1.0 eq.) was suspended in THE (2.8 mL) that contained TEA (39 μL, 0.28 mmol, 1.0 eq.), and cinnamaldehyde (42 μL, 0.34 mmol, 1.2 eq.). After stirring at RT for 10 min, STAB-H (148 mg, 0.699 mmol, 2.5 eq.) was added and the reaction mixture was stirred overnight. MeOH was added to terminate the reaction and the volatiles were removed by in vacuo. The resulting solids were suspended in an aqueous solution of NaOH (2M) and extracted with DCM (3 times). The combined organic layers were dried over anhydrous Na₂SO₄, filtered and concentrated in vacuo. The crude product was purified by NP column chromatography (using a linear gradient of 10-60% (EtOAc:MeOH:TEA, 90:5:5, v:v:v %)/cyclohexane) to obtain the title compound as an off-white solid (68 mg, 56%). LC/MS (acidic mode) Rₜ 3.94 min, purity >96%, [M+H]⁺ calculated 438.20, found 438.20. ¹H NMR (500 MHz, DMSO-d6) δ 8.89 (t, J=5.9 Hz, 1H), 8.05 (ddd, J=8.7, 8.7, 6.4 Hz, 1H), 7.57 (ddd, J=11.5, 9.2, 2.5 Hz, 1H), 7.47-7.39 (m, 2H), 7.36-7.28 (m, 3H), 7.26-7.20 (m, 1H), 7.15 (d, J=3.0 Hz, 1H), 6.50 (d, J=15.9 Hz, 1H), 6.29 (dt, J=15.9, 6.5 Hz, 1H), 3.16 (dd, J=6.4, 6.4 Hz, 2H), 3.09-3.02 (m, 2H), 2.90-2.82 (m, 2H), 1.93-1.84 (m, 2H), 1.81-1.57 (m, 2H), 1.60-1.51 (m, 1H), 1.25-1.13 (m, 2H). ¹³C NMR (126 MHz, DMSO-d6) δ 164.0, 163.6 (dd, J=251.9, 12.5 Hz), 159.6, 159.0 (dd, J=255.2, 12.7 Hz), 158.2, 136.7, 131.6, 129.6 (dd, J=10.3, 3.3 Hz), 128.6, 127.7, 127.3, 126.2, 113.0 (dd, J=22.0, 3.5 Hz), 112.0-110.9 (m), 105.5 (dd, J=26.0, 26.0 Hz), 102.5 (d, J=8.3 Hz), 60.5, 53.0, 44.4, 35.6, 29.8. HRMS m/z [M+H]⁺ C₂₅H₂₆F₂N₃O₂⁺ calculated 438.1988, found 438.2007.

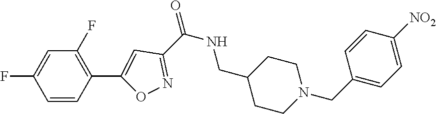 [SMILES: O=C(NCC1CCN(Cc2ccc([N+](=O)[O-])cc2)CC1)c1cc(-c2ccc(F)cc2F)on1]

### 5-(2,4-difluorophenyl)-N-((1-(4-nitrobenzyl)piperidin-4-yl)methyl)isoxazole-3-carboxamide (43)

This compound was synthesised according to general procedure D. 5-(2,4-difluorophenyl)-N-(piperidin-4-ylmethyl)isoxazole-3-carboxamide hydrochloride (100 mg, 0.279 mmol, 1.0 eq.), TEA (39 μL, 0.28 mmol, 1.0 eq.), 4-nitrobenzaldehyde (51 mg, 0.34 mmol, 1.2 eq.) and STAB-H (148 mg, 0.699 mmol, 2.5 eq.) were used. Additional 4-nitrobenzaldehyde (25 mg, 0.17 mmol, 0.6 eq.) and STAB-H (71 mg, 0.34 mmol, 1.2 eq. each) were added drive the reaction to completion. The crude product was purified by NP column chromatography (using a linear gradient of 10-50% (EtOAc:MeOH:TEA, 90:5:5, v:v:v %)/cyclohexane). The obtained solid was triturated with MeOH to afford the title compound as a white solid (32 mg, 25%). LC/MS (acidic mode) Rₜ 3.41 min, purity 98%, [M+H]⁺ calculated 457.17, found 457.20. ¹H NMR (500 MHz, DMSO-d6) δ 8.89 (t, J=5.9 Hz, 1H), 8.21-8.16 (m, 2H), 8.05 (ddd, J=8.7, 8.7, 6.4 Hz, 1H), 7.62-7.55 (m, 3H), 7.32 (ddd, J=8.4, 8.4, 2.4 Hz, 1H), 7.15 (d, J=2.9 Hz, 1H), 3.58 (s, 2H), 3.17 (dd, J=6.4, 6.4 Hz, 2H), 2.82-2.74 (m, 2H), 2.00-1.91 (m, 2H), 1.68-1.61 (m, 2H), 1.61-1.53 (m, 1H), 1.26-1.15 (m, 2H). ¹³C NMR (126 MHz, DMSO-d6) δ 164.5, 164.1 (dd, J=251.9, 12.3 Hz), 160.1, 159.5 (dd, J=255.3, 12.9 Hz), 158.7, 147.8, 147.0, 130.1 (dd, J=10.4, 3.3 Hz), 130.0, 123.8, 113.5 (dd, J=21.9, 3.5 Hz), 112.4-111.5 (m), 106.0 (dd, J=25.9, 25.9 Hz), 102.9 (d, J=8.5 Hz), 61.8, 53.4, 44.9, 35.9, 30.2. HRMS m/z [M+H]⁺ C₂₃H₂₃F₂N₄O₄⁺ calculated 457.1682, found 457.1702.

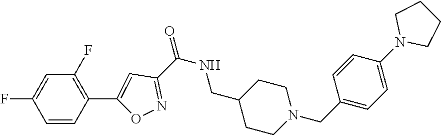 [SMILES: O=C(NCC1CCN(Cc2ccc(N3CCCC3)cc2)CC1)c1cc(-c2ccc(F)cc2F)on1]

### 5-(2,4-difluorophenyl)-N-((1-(4-(pyrrolidin-1-yl)benzyl)piperidin-4-yl)methyl)isoxazole-3-carboxamide (44)

This compound was synthesised according to general procedure D. 5-(2,4-difluorophenyl)-N-(piperidin-4-ylmethyl)isoxazole-3-carboxamide hydrochloride (100 mg, 0.279 mmol, 1.0 eq.), TEA (39 μL, 0.28 mmol, 1.0 eq.), 4-(pyrrolidine-1-yl)benzaldehyde (59 mg, 0.34 mmol, 1.2 eq.) and STAB-H (148 mg, 0.699 mmol, 2.5 eq.) were used. The crude product was purified by NP column chromatography (using a linear gradient of 30-70% (EtOAc:MeOH:TEA, 90:5:5, v:v:v %)/cyclohexane) to obtain the title compound as a white solid (68 mg, 50%). LC/MS (acidic mode) Rₜ 3.76 min, purity >98%, [M+H]⁺ calculated 481.24, found 481.30. ¹H NMR (600 MHz, DMSO-d6) δ 8.86 (t, J=6.0 Hz, 1H), 8.05 (ddd, J=8.7, 8.7, 6.3 Hz, 1H), 7.57 (ddd, J=11.5, 9.2, 2.5 Hz, 1H), 7.32 (ddd, J=8.4, 8.4, 2.5 Hz, 1H), 7.14 (d, J=2.9 Hz, 1H), 7.06-7.03 (m, 2H), 6.50-6.41 (m, 2H), 3.29 (s, 2H), 3.21-3.16 (m, 4H), 3.15 (dd, J=6.4, 6.4 Hz, 2H), 2.80-2.74 (m, 2H), 1.95-1.90 (m, 4H), 1.86-1.79 (m, 2H), 1.72-1.58 (m, 2H), 1.64-1.47 (m, 1H), 1.19-1.10 (m, 2H). ¹³C NMR (151 MHz, DMSO-d6) δ 164.0 (d, J=2.7 Hz), 163.6 (dd, J=252.0, 12.4 Hz), 159.6, 159.0 (dd, J=254.8, 13.2 Hz), 158.2, 146.8, 129.8, 129.6 (dd, J=10.5, 3.3 Hz), 124.6, 113.0 (dd, J=22.0, 3.3 Hz), 111.5 (dd, J=12.4, 3.8 Hz), 111.2, 105.5 (dd, J=26.0, 26.0 Hz), 102.5 (d, J=8.3 Hz), 62.2, 52.7, 47.3, 44.5, 35.6, 29.7, 24.9. HRMS m/z [M+H-p-(pyrrolidine-1-yl)Bn]⁺ C₁₆H₁₈F₂N₃O₂⁺ calculated 322.1362, found 322.1379.

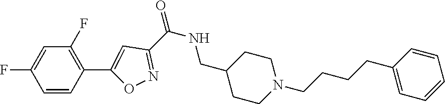 [SMILES: O=C(NCC1CCN(CCCCc2ccccc2)CC1)c1cc(-c2ccc(F)cc2F)on1]

### 5-(2,4-difluorophenyl)-N-((1-(4-phenylbutyl)piperidin-4-yl)methyl)isoxazole-3-carboxamide (45)

This compound was synthesised according to general procedure C. 5-(2,4-difluorophenyl)-N-(piperidin-4-ylmethyl)isoxazole-3-carboxamide hydrochloride (100 mg, 0.279 mmol, 1.0 eq.), 1-chloro-4-phenylbutane (51 μL, 0.31 mmol, 1.1 eq.) and K₂CO₃ (77 mg, 0.56 mmol, 2.0 eq.) were used. The reaction mixture was stirred overnight at RT. Additional 1-chloro-4-phenylbutane was added (1.5 eq.) and the mixture was stirred for another 24 h. Subsequently, the reaction mixture was heated to 50° C. and stirred at that temperature overnight. The volatiles were evaporated in vacuo and the residue was suspended in an aqueous solution of NaOH (2M). The formed suspension was extracted with DCM (three times). The combined organic layers were dried over anhydrous Na₂SO₄, filtered and concentrated in vacuo. The crude product was purified by NP column chromatography (using a linear gradient of 10-50% (EtOAc:MeOH:TEA, 90:5:5, v:v:v %)/cyclohexane) to obtain the title compound as an off-white solid (40 mg, 32%). LC/MS (acidic mode) Rₜ 3.73 min, purity 97%, [M+H]⁺ calculated 454.23, found 454.20. ¹H NMR (500 MHz, DMSO-d6) δ 8.87 (t, J=6.0 Hz, 1H), 8.05 (ddd, J=8.7, 8.7, 6.2 Hz, 1H), 7.57 (ddd, J=11.5, 9.2, 2.6 Hz, 1H), 7.32 (ddd, J=8.5, 8.5, 2.6 Hz, 1H), 7.29-7.23 (m, 2H), 7.19-7.13 (m, 4H), 3.14 (dd, J=6.4, 6.4 Hz, 2H), 2.83-2.75 (m, 2H), 2.60-2.52 (m, 2H), 2.27-2.19 (m, 2H), 1.82-1.74 (m, 2H), 1.65-1.58 (m, 2H), 1.58-1.50 (m, 3H), 1.44-1.36 (m, 2H), 1.19-1.08 (m, 2H). ¹³C NMR (126 MHz, DMSO-d6) δ 164.0, 163.6 (dd, J=252.1, 12.4 Hz), 159.6, 159.0 (dd, J=255.4, 12.9 Hz), 158.2, 142.3, 129.6 (dd, J=10.3, 3.4 Hz), 128.2, 128.2, 125.6, 113.0 (dd, J=22.1, 3.5 Hz), 111.9-110.9 (m), 105.5 (dd, J=26.0, 26.0 Hz), 102.4 (d, J=8.4 Hz), 58.0, 53.1, 44.5, 35.7, 35.0, 29.8, 28.9, 26.1. HRMS m/z [M+H]⁺ C₂₆H₃₀F₂N₃O₂⁺ calculated 454.2301, found 454.2307.

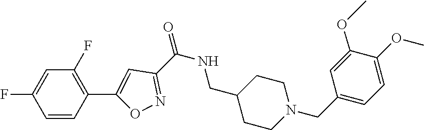 [SMILES: COc1ccc(CN2CCC(CNC(=O)c3cc(-c4ccc(F)cc4F)on3)CC2)cc1OC]

### 5-(2,4-difluorophenyl)-N-((1-(3,4-dimethoxybenzyl)piperidin-4-yl)methyl)isoxazole-3-carboxamide (46)

This compound was synthesised according to general procedure D. 5-(2,4-difluorophenyl)-N-(piperidin-4-ylmethyl)isoxazole-3-carboxamide hydrochloride (100 mg, 0.279 mmol, 1.0 eq.), TEA (39 μL, 0.28 mmol, 1.0 eq.), 3,4-dimethoxybenzaldehyde (56 mg, 0.34 mmol, 1.2 eq.) and STAB-H (148 mg, 0.699 mmol, 2.5 eq.) were used. The crude product was purified by NP column chromatography (using a linear gradient of 10-50% (EtOAc:MeOH:TEA, 90:5:5, v:v:v %)/cyclohexane) to obtain the title compound as a white solid (100 mg, 76%). LC/MS (acidic mode) Rₜ 3.41 min, purity 98%, [M+H]⁺ calculated 472.20, found 472.20. ¹H NMR (500 MHz, DMSO-d6) δ 8.88 (t, J=5.9 Hz, 1H), 8.05 (ddd, J=8.7, 8.7, 6.4 Hz, 1H), 7.58 (ddd, J=11.6, 9.2, 2.5 Hz, 1H), 7.35-7.29 (m, 1H), 7.15 (d, J=2.9 Hz, 1H), 6.88-6.84 (m, 2H), 6.79-6.75 (m, 1H), 3.73 (s, 3H), 3.72 (s, 3H), 3.35 (s, 2H), 3.16 (dd, J=6.4, 6.4 Hz, 2H), 2.81-2.76 (m, 2H), 1.89-1.81 (m, 2H), 1.66-1.60 (m, 2H), 1.60-1.51 (m, 1H), 1.23-1.12 (m, 2H). ¹³C NMR (126 MHz, DMSO-d6) δ 164.0, 163.6 (dd, J=252.1, 12.6 Hz), 159.6, 159.0 (dd, J=255.2, 12.8 Hz), 158.2, 148.5, 147.7, 131.1, 129.6 (dd, J=10.4, 3.3 Hz), 120.7, 113.0 (dd, J=22.0, 3.5 Hz), 112.3, 111.5, 111.5, 105.5 (dd, J=26.0, 26.0 Hz), 102.5 (d, J=8.3 Hz), 62.2, 55.5, 55.4, 52.8, 44.4, 35.6, 29.7. HRMS m/z [M+H]⁺ C₂₅H₂₈F₂N₃O₄⁺ calculated 472.2042, found 472.2055.

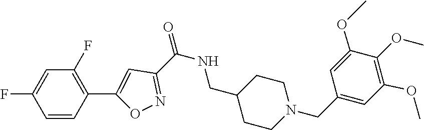 [SMILES: COc1cc(CN2CCC(CNC(=O)c3cc(-c4ccc(F)cc4F)on3)CC2)cc(OC)c1OC]

### 5-(2,4-difluorophenyl)-N-((1-(3,4,5-trimethoxybenzyl)piperidin-4-yl)methyl)isoxazole-3-carboxamide(47)

This compound was synthesised according to general procedure D. 5-(2,4-difluorophenyl)-N-(piperidin-4-ylmethyl)isoxazole-3-carboxamide hydrochloride (100 mg, 0.279 mmol, 1.0 eq.), TEA (39 μL, 0.28 mmol, 1.0 eq.), 3,4,5-trimethoxybenzaldehyde (66 mg, 0.34 mmol, 1.2 eq.) and STAB-H (148 mg, 0.699 mmol, 2.5 eq.) were used. The crude product was purified by NP column chromatography (using a linear gradient of 10-50% (EtOAc:MeOH:TEA, 90:5:5, v:v:v %)/cyclohexane) to obtain the title compound as a white solid (110 mg, 79%). LC/MS (acidic mode) Rₜ 3.51 min, purity >98%, [M+H]⁺ calculated 502.21, found 502.20. ¹H NMR (500 MHz, DMSO-d6) δ 8.89 (t, J=5.9 Hz, 1H), 8.05 (ddd, J=8.7, 8.7, 6.3 Hz, 1H), 7.58 (ddd, J=11.6, 9.2, 2.5 Hz, 1H), 7.35-7.30 (m, 1H), 7.15 (d, J=3.0 Hz, 1H), 6.58 (s, 2H), 3.75 (s, 6H), 3.63 (s, 3H), 3.36 (s, 2H), 3.17 (dd, J=6.4, 6.4 Hz, 2H), 2.83-2.78 (m, 2H), 1.91-1.83 (m, 2H), 1.68-1.60 (m, 2H), 1.60-1.51 (m, 1H), 1.25-1.13 (m, 2H). ¹³C NMR (126 MHz, DMSO-d6) δ 164.0, 163.6 (dd, J=252.0, 12.1 Hz), 159.6, 159.0 (dd, J=255.3, 12.8 Hz), 158.2, 152.7, 136.2, 134.5, 129.6 (dd, J=10.4, 3.4 Hz), 113.0 (dd, J=22.0, 3.5 Hz), 112.9-110.3 (m), 105.5, 105.5 (dd, J=26.0, 26.0 Hz), 102.5 (d, J=8.4 Hz), 62.5, 59.9, 55.8, 52.9, 44.4, 35.6, 29.7. HRMS m/z [M+H]⁺ C₂₆H₃₀F₂N₃O₅ calculated 502.2148, found 502.2166.

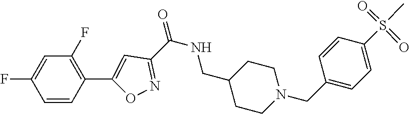 [SMILES: CS(=O)(=O)c1ccc(CN2CCC(CNC(=O)c3cc(-c4ccc(F)cc4F)on3)CC2)cc1]

### 5-(2,4-difluorophenyl)-N-((1-(4-(methylsulfonyl)benzyl)piperidin-4-yl)methyl)isoxazole-3-carboxamide (48)

5-(2,4-difluorophenyl)-N-(piperidin-4-ylmethyl)isoxazole-3-carboxamide hydrochloride (100 mg, 0.279 mmol, 1.0 eq.) was suspended in DMF (2.8 mL). K₂CO₃ (77 mg, 0.56 mmol, 2.0 eq.) and 1-(chloromethyl)-4-(methylsulfonyl)benzene (63 mg, 0.31 mmol, 1.1 eq.) were added. The reaction mixture was stirred overnight at RT and then concentrated in vacuo. The resulting crude product was purified by NP column chromatography (using a linear gradient of 10-50% (EtOAc:MeOH:TEA, 90:5:5, v:v:v %)/cyclohexane) to obtain the title compound as a white solid (67 mg, 49%). LC/MS (acidic mode) Rₜ 3.22 min, purity >99%, [M+H]⁺ calculated 490.16, found 490.15. ¹H NMR (500 MHz, DMSO-d6) δ 8.89 (t, J=6.0 Hz, 1H), 8.05 (ddd, J=8.7, 8.7, 6.4 Hz, 1H), 7.90-7.83 (m, 2H), 7.61-7.52 (m, 3H), 7.37-7.29 (m, 1H), 7.15 (d, J=2.9 Hz, 1H), 3.55 (s, 2H), 3.20 (s, 3H), 3.17 (dd, J=6.4, 6.4 Hz, 2H), 2.82-2.74 (m, 2H), 1.98-1.90 (m, 2H), 1.68-1.62 (m, 2H), 1.62-1.53 (m, 1H), 1.26-1.15 (m, 2H). ¹³C NMR (126 MHz, DMSO-d6) δ 164.0, 163.6 (dd, J=252.0, 12.5 Hz), 159.6, 159.0 (dd, J=255.3, 12.8 Hz), 158.2, 145.2, 139.3, 129.6 (dd, J=10.4, 3.3 Hz), 129.3, 126.9, 113.0 (dd, J=21.9, 3.5 Hz), 111.5 (dd, J=12.5, 3.8 Hz), 105.5 (dd, J=25.9, 25.9 Hz), 102.5 (d, J=8.5 Hz), 61.6, 52.9, 44.4, 43.6, 35.4, 29.7. HRMS m/z [M+H]⁺ C₂₄H₂₆F₂N₃O₄S⁺ calculated 490.1607, found 490.1618.

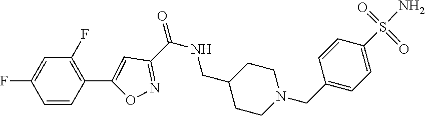 [SMILES: NS(=O)(=O)c1ccc(CN2CCC(CNC(=O)c3cc(-c4ccc(F)cc4F)on3)CC2)cc1]

### 5-(2,4-difluorophenyl)-N-((1-(4-sulfamoylbenzyl)piperidin-4-yl)methyl)isoxazole-3-carboxamide (49)

5-(2,4-difluorophenyl)-N-(piperidin-4-ylmethyl)isoxazole-3-carboxamide hydrochloride (200 mg, 0.559 mmol, 1.0 eq.) was suspended in DMF (2.8 mL) and DIPEA (195 mg, 1.12 mmol, 2.0 eq.) and 4-(bromomethyl)benzenesulfonamide (70 mg, 0.61 mmol, 1.1 eq.) were added. The reaction mixture was stirred overnight at RT. The reaction mixture was poured into an ice-cold aqueous solution of NaOH (2M) and extracted with DCM (3 times). The combined organic layers were dried over anhydrous Na₂SO₄, filtered and concentrated in vacuo. The crude product was purified by RP column chromatography (using a linear gradient of 10-70% ACN/H₂O with 0.1% FA) to obtain the title compound as a white solid (127 mg, 46%). LC/MS (acidic mode) Rₜ 3.08 min, purity >97%, [M+H]⁺ calculated 491.16, found 491.25. ¹H NMR (600 MHz, DMSO-d6) δ 8.87 (t, J=5.9 Hz, 1H), 8.05 (ddd, J=8.7, 8.6, 6.3 Hz, 1H), 7.79-7.74 (m, 2H), 7.57 (ddd, J=11.5, 9.2, 2.6 Hz, 1H), 7.47 (d, J=8.1 Hz, 2H), 7.35-7.26 (m, 3H), 7.15 (d, J=2.9 Hz, 1H), 3.51 (s, 2H), 3.17 (dd, J=6.4, 6.4 Hz, 2H), 2.83-2.72 (m, 2H), 1.97-1.89 (m, 2H), 1.69-1.61 (m, 2H), 1.61-1.53 (m, 1H), 1.24-1.15 (m, 2H). ¹³C NMR (151 MHz, DMSO-d6) δ 164.0 (d, J=2.7 Hz), 163.6 (dd, J=252.0, 12.3 Hz), 159.6, 159.0 (dd, J=255.3, 12.8 Hz), 158.2, 143.1, 142.6, 129.6 (dd, J=10.2, 3.2 Hz), 128.9, 125.6, 113.0 (dd, J=22.2, 3.5 Hz), 111.5 (dd, J=12.4, 3.8 Hz), 105.5 (dd, J=26.0, 26.0 Hz), 102.5 (d, J=8.5 Hz), 61.7, 52.9, 44.4, 35.4, 29.7. HRMS m/z [M+H]⁺ C₂₃H₂₅F₂N₄O₄S⁺ calculated 491.1559, found 491.1578.

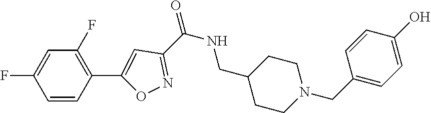 [SMILES: O=C(NCC1CCN(Cc2ccc(O)cc2)CC1)c1cc(-c2ccc(F)cc2F)on1]

### 5-(2,4-difluorophenyl)-N-((1-(4-hydroxybenzyl)piperidin-4-yl)methyl)isoxazole-3-carboxamide (50)

5-(2,4-difluorophenyl)-N-(piperidin-4-ylmethyl)isoxazole-3-carboxamide hydrochloride (100 mg, 0.279 mmol, 1.0 eq.) was suspended in THE (2.8 mL) and TEA (39 μL, 0.28 mmol, 1.0 eq.) and 4-hydroxybenzaldehyde (41 mg, 0.34 mmol, 1.2 eq.) were added. The mixture was stirred at RT for 10 min. Subsequently, STAB-H (148 mg, 0.699 mmol, 2.5 eq.) was added and the reaction mixture was stirred overnight at RT. Additional 4-hydroxybenzaldehyde (20 mg, 0.17 mmol, 1.2 eq.) and STAB-H (71 mg, 0.34 mmol, 1.2 eq.) were added to drive the reaction to completion. MeOH was added to terminate the reaction and the volatiles were removed in vacuo. The crude product was purified by NP column chromatography (using a linear gradient of 10-50% (EtOAc:MeOH:TEA, 90:5:5, v:v:v %)/cyclohexane). The combined product fractions were triturated with MeOH to obtain the title compound as a white solid (67 mg, 49%). LC/MS (acidic mode) Rₜ 3.21 min, purity 98%, [M+H]⁺ calculated 428.18, found 428.20. ¹H NMR (500 MHz, DMSO-d6) δ 9.29-9.17 (m, 1H), 8.94-8.78 (m, 1H), 8.11-7.99 (m, 1H), 7.64-7.51 (m, 1H), 7.38-7.27 (m, 1H), 7.14 (d, J=3.2 Hz, 1H), 7.09-7.01 (m, 2H), 6.74-6.62 (m, 2H), 3.29 (s, 2H), 3.21-3.10 (m, 2H), 2.81-2.72 (m, 2H), 1.82 (t, J=11.5 Hz, 2H), 1.66-1.58 (m, 2H), 1.58-1.50 (m, 1H), 1.22-1.08 (m, 2H). ¹³C NMR (126 MHz, DMSO-d6) δ 164.0, 163.6 (dd, J=251.7, 12.2 Hz), 159.6, 159.0 (dd, J=255.2, 12.7 Hz), 158.2, 156.2, 129.9, 129.6 (dd, J=10.4, 2.8 Hz), 128.6, 114.8, 113.0 (dd, J=22.3, 2.6 Hz), 111.5 (dd, J=12.4, 3.6 Hz), 105.5 (dd, J=26.0, 26.0 Hz), 102.5 (d, J=8.5 Hz), 62.0, 52.7, 44.4, 35.6, 29.7. HRMS m/z [M+H]⁺ C₂₃H₂₄F₂N₃O₃⁺ calculated 428.1780, found 428.1784.

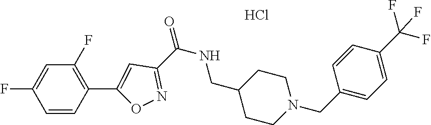 [SMILES: O=C(NCC1CCN(Cc2ccc(C(F)(F)F)cc2)CC1)c1cc(-c2ccc(F)cc2F)on1 ; Cl]

### 5-(2,4-difluorophenyl)-N-((1-(4-(trifluoromethyl)benzyl)piperidin-4-yl)methyl)isoxazole-3-carboxamide hydrochloride(51)

5-(2,4-difluorophenyl)-N-(piperidin-4-ylmethyl)isoxazole-3-carboxamide hydrochloride (200 mg, 0.559 mmol, 1.0 eq.) was suspended in THE (2.8 mL) and TEA (78 μL, 0.56 mmol, 1.0 eq.) and 4-(trifluoromethyl)benzaldehyde (117 mg, 0.671 mmol, 1.2 eq.) were added. The mixture was stirred at RT for 10 min. Subsequently, STAB-H (296 mg, 1.40 mmol, 2.5 eq.) was added and the reaction mixture was stirred overnight at RT. MeOH was added to terminate the reaction and the volatiles were removed in vacuo. The resulting solid were suspended in an ice-cold aqueous solution of NaOH (2M). The solids were collected by vacuum filtration and dissolved in MeOH. An excess of 4M HCl in dioxane was added. The volatiles were removed in vacuo to obtain the HCl salt. The resulting salt was triturated with acetone to obtain the title compound as a yellow solid (100 mg, 35%). LC/MS (acidic mode) Rₜ 3.63 min, purity >99%, [M+H]⁺ calculated 480.17, found 480.20. ¹H NMR (500 MHz, DMSO-d6) δ 11.17-10.89 (m, 1H), 9.03 (t, J=6.0 Hz, 1H), 8.05 (ddd, J=8.7, 8.6, 6.3 Hz, 1H), 7.90-7.80 (m, 4H), 7.58 (ddd, J=11.5, 9.2, 2.5 Hz, 1H), 7.38-7.29 (m, 1H), 7.20-7.14 (m, 1H), 4.42 (d, J=5.5 Hz, 2H), 3.34-3.28 (m, 2H), 3.17 (dd, J=6.2, 6.2 Hz, 2H), 2.96-2.84 (m, 2H), 1.88-1.77 (m, 3H), 1.66-1.53 (m, 2H). Cis-trans mixture observed. ¹³C NMR (126 MHz, DMSO-d6) δ 164.6, 164.1 (dd, J=252.2, 12.5 Hz), 160.0, 161.3-157.5 (m), 158.8, 135.0, 132.8, 130.3, 130.1 (dd, J=10.3, 3.5 Hz), 126.0 (d, J=4.2 Hz), 124.5 (q, J=272.3 Hz), 113.5 (dd, J=22.1, 3.4 Hz), 111.9 (dd, J=12.4, 3.8 Hz), 106.0 (dd, J=26.0, 26.0 Hz), 103.0 (d, J=8.2 Hz), 58.6, 51.8, 44.1, 34.0, 27.0. HRMS m/z [M+H]⁺ C₂₄H₂₃F₅N₃O₂⁺ calculated 480.1705, found 480.1710.

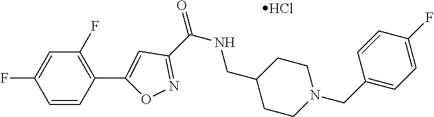 [SMILES: O=C(NCC1CCN(Cc2ccc(F)cc2)CC1)c1cc(-c2ccc(F)cc2F)on1 ; Cl]

### 5-(2,4-difluorophenyl)-N-((1-(4-fluorobenzyl)piperidin-4-yl)methyl)isoxazole-3-carboxamide hydrochloride (52)

5-(2,4-difluorophenyl)-N-(piperidin-4-ylmethyl)isoxazole-3-carboxamide hydrochloride (200 mg, 0.559 mmol, 1.0 eq.) was suspended in THE (2.8 mL) and TEA (78 μL, 0.56 mmol, 1.0 eq.) and 4-fluorobenzaldehyde (83 mg, 0.67 mmol, 1.2 eq.) were added. The mixture was stirred at RT for 10 min. Subsequently, STAB-H (296 mg, 1.40 mmol, 2.5 eq.) was added and the reaction mixture was stirred overnight at RT. MeOH was added to terminate the reaction and the volatiles were removed in vacuo. The resulting solid were suspended in an ice-cold aqueous solution of NaOH (2M). The solids were collected by vacuum filtration and dissolved in MeOH. An excess of 4M HCl in dioxane was added. The volatiles were removed in vacuo to obtain the HCl salt. The resulting salt was triturated with acetone to obtain the title compound as a yellow solid (170 mg, 65%). LC/MS (acidic mode) Rₜ 3.44 min, purity 95%, [M+H]⁺ calculated 430.17, found 430.15. ¹H NMR (500 MHz, DMSO-d6) δ 10.83-10.53 (m, 1H), 9.09-8.97 (m, 1H), 8.05 (ddd, J=8.7, 8.6, 6.3 Hz, 1H), 7.73-7.62 (m, 2H), 7.58 (ddd, J=11.5, 9.2, 2.5 Hz, 1H), 7.36-7.25 (m, 3H), 7.20-7.13 (m, 1H), 4.24 (d, J=5.5 Hz, 2H), 3.33-3.26 (m, 2H), 3.17 (dd, J=6.1, 6.1 Hz, 2H), 2.92-2.78 (m, 2H), 1.93-1.77 (m, 3H), 1.63-1.49 (m, 2H). Cis-trans mixture observed. ¹³C NMR (126 MHz, DMSO-d6) δ 164.1, 163.7 (dd, J=252.0, 12.3 Hz), 162.7 (d, J=246.3 Hz), 159.5, 159.0 (dd, J=255.4, 12.8 Hz), 158.3, 133.8 (d, J=8.6 Hz), 129.6 (dd, J=10.4, 3.3 Hz), 126.2 (d, J=3.2 Hz), 115.6 (d, J=21.7 Hz), 113.0 (dd, J=22.2, 3.6 Hz), 111.4 (dd, J=12.5, 3.7 Hz), 105.5 (dd, J=25.9, 25.9 Hz), 102.5 (d, J=8.4 Hz), 58.0, 51.0, 43.6, 33.5, 26.5. HRMS m/z [M+H]⁺ C₂₃H₂₃F₃N₃O₂⁺ calculated 430.1737, found 430.1744.

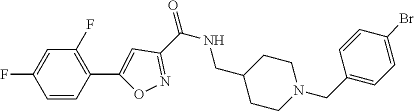 [SMILES: O=C(NCC1CCN(Cc2ccc(Br)cc2)CC1)c1cc(-c2ccc(F)cc2F)on1]

### N-((1-(4-bromobenzyl)piperidin-4-yl)methyl)-5-(2,4-difluorophenyl)isoxazole-3-carboxamide (53)

This compound was synthesised according to general procedure C. 5-(2,4-difluorophenyl)-N-(piperidin-4-ylmethyl)isoxazole-3-carboxamide hydrochloride (200 mg, 0.559 mmol, 1.0 eq.), K₂CO₃ (155 mg, 1.12 mmol, 2.0 eq.) and 4-bromobenzyl bromide (154 mg, 0.615 mmol, 1.1 eq.) were used. The crude product was purified by NP column chromatography (using a linear gradient of 10-50% (EtOAc:MeOH:TEA, 90:5:5, v:v:v %)/cyclohexane) to obtain the title compound as a white solid (100 mg, 56%). LC/MS (acidic mode) Rₜ 3.57 min, purity 99%, [M+H]⁺ calculated 490.09; 492.09, found 490.10; 492.15. ¹H NMR (500 MHz, DMSO-d6) δ 8.88 (t, J=6.0 Hz, 1H), 8.05 (dd, J=8.7, 8.7, 6.4 Hz, 1H), 7.57 (ddd, J=11.5, 9.3, 2.5 Hz, 1H), 7.52-7.47 (m, 2H), 7.33 (ddd, J=8.6, 8.5, 2.5 Hz, 1H), 7.27-7.22 (m, 2H), 7.15 (d, J=2.9 Hz, 1H), 3.40 (s, 2H), 3.16 (dd, J=6.4, 6.4 Hz, 2H), 2.80-2.72 (m, 2H), 1.93-1.84 (m, 2H), 1.66-1.60 (m, 2H), 1.60-1.52 (m, 1H), 1.24-1.12 (m, 2H). ¹³C NMR (126 MHz, DMSO-d6) δ 164.0, 163.6 (dd, J=252.1, 12.2 Hz), 159.6, 159.0 (dd, J=255.3, 13.0 Hz), 158.2, 138.3, 131.0, 130.8, 129.6 (dd, J=10.3, 3.3 Hz), 119.7, 113.0 (dd, J=22.2, 3.5 Hz), 111.8-111.4 (m), 105.5 (dd, J=25.8, 25.8 Hz), 102.5 (d, J=8.4 Hz), 61.5, 52.8, 44.4, 35.5, 29.7. HRMS m/z [M+H]⁺ C₂₃H₂₃BrF2N₃O₂⁺ calculated 490.0936; 492.0916, found 490.0957; 492.0948.

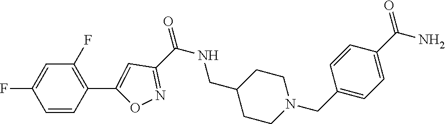 [SMILES: NC(=O)c1ccc(CN2CCC(CNC(=O)c3cc(-c4ccc(F)cc4F)on3)CC2)cc1]

### N-((1-(4-carbamoylbenzyl)piperidin-4-yl)methyl)-5-(2,4-difluorophenyl)isoxazole-3-carboxamide (54)

5-(2,4-difluorophenyl)-N-(piperidin-4-ylmethyl)isoxazole-3-carboxamide hydrochloride (200 mg, 0.559 mmol, 1.0 eq.) was suspended in DMF (5.6 mL) and DIPEA (195 μL, 1.12 mmol, 2.0 eq.) and 4-(chloromethyl)benzamide (104 mg, 0.615 mmol, 1.1 eq.) were added. The reaction mixture was stirred overnight at RT. Subsequently, the reaction mixture was poured into an ice-cold aqueous solution of NaOH (2M). The resulting aqueous phase was extracted thrice with DCM. The DCM was dried over anhydrous Na₂SO₄, filtered and concentrated in vacuo to obtain the crude product. The crude product was purified by RP column chromatography (using a linear gradient of 10-70% ACN/H₂O with 0.1% FA). The product fractions were combined, basified with an aqueous solution of NaOH (2M) and extracted three times with DCM. The combined DCM layers were dried over anhydrous Na₂SO₄, filtered and concentrated in vacuo to obtain the title compound as a white solid (130 mg, 51%). LC/MS (acidic mode) Rₜ 2.96 min, purity >96%, [M+H]⁺ calculated 455.19, found 455.25. ¹H NMR (600 MHz, DMSO-d6) δ 10.68 (s, 1H), 9.01 (t, J=6.0 Hz, 1H), 8.08-8.01 (m, 2H), 7.93 (d, J=7.8 Hz, 2H), 7.68 (d, J=8.3 Hz, 2H), 7.57 (ddd, J=11.5, 9.2, 2.5 Hz, 1H), 7.46 (s, 1H), 7.33 (ddd, J=8.5, 8.5, 2.6 Hz, 1H), 7.16 (d, J=2.9 Hz, 1H), 4.29 (d, J=5.2 Hz, 2H), 3.34-3.27 (m, 2H), 3.17 (dd, J=6.2, 6.2 Hz, 2H), 2.95-2.83 (m, 2H), 1.94-1.68 (m, 3H), 1.65-1.50 (m, 2H). Cis-trans mixture observed ¹³C NMR (151 MHz, DMSO-d6) δ 167.3, 164.1 (d, J=2.8 Hz), 163.7 (dd, J=252.3, 12.4 Hz), 159.5, 159.0 (dd, J=255.3, 12.9 Hz), 158.4, 135.0, 132.9, 131.3, 129.6 (dd, J=10.4, 3.3 Hz), 127.8, 113.0 (dd, J=22.2, 3.4 Hz), 111.4 (dd, J=12.7, 3.9 Hz), 105.5 (dd, J=26.0, 26.0 Hz), 102.5 (d, J=8.2 Hz), 58.5, 51.3, 43.6, 33.5, 26.6. HRMS m/z [M+H]⁺ C₂₄H₂₅F₂N₄O₃⁺ calculated 455.1889, found 455.1910.

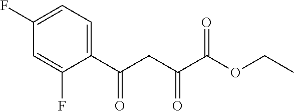 [SMILES: CCOC(=O)C(=O)CC(=O)c1ccc(F)cc1F]

### Ethyl 4-(2,4-difluorophenyl)-2,4-dioxobutanoate (55)

To a solution of 1-(2,4-difluorophenyl)ethan-1-one (100 g, 0.640 mol, 1.0 eq.) in THE (430 mL) was added diethyl oxalate (104 mL, 0.769 mol, 1.2 eq.). To the resulting mixture, NaOtBu (67.7 g, 769 mol, 1.0 eq.) was added in small portions. The mixture was left at RT for 16 h, during which the mixture solidified owing to the sodium salt of the Claisen adduct. The resulting suspension was filtered, and the solid product was washed with THF. The solid material was transferred to an Erlenmeyer flask and treated with aq. HCl (1 M) forming a yellow-white precipitate. The precipitate was filtered and dried in a vacuum oven yielding the title compound as an off-white solid (150 g, 0.585 mol, 91%). LCMS (acidic mode) Rₜ 4.81 min, purity 94% [M+H]1 calculated 257.06, not detected. ¹H NMR (500 MHz, DMSO-d6) δ 8.07-7.88 (m, 1H), 7.56-7.39 (m, 1H), 7.37-7.22 (m, 1H), 6.88 (s, 1H), 4.30 (q, J=7.0 Hz, 2H), 1.29 (t, J=7.1 Hz, 3H).

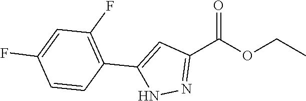 [SMILES: CCOC(=O)c1cc(-c2ccc(F)cc2F)[nH]n1]

### Ethyl 5-(2,4-difluorophenyl)-1H-pyrazole-3-carboxylate (56)

To a stirred solution of di-oxo compound 55 (1.20 g, 4.68 mmol), H₂NNH₂·H₂O (0.258 g, 5.15 mmol) and EtOH (20 mL) were added. The mixture was heated at reflux for 1 hr. The mixture was cooled and poured into water. The precipitated solid was collected by filtration, washed with water, and dried to give the title compound as an off-white solid (1.05 g, 4.16 mmol, 89%). LCMS (acidic mode) Rₜ 4.30 min, purity >95% [M+H]⁺ calculated 253.08, found 252.95. ¹H NMR (500 MHz, DMSO-d6) δ 8.11-7.83 (m, 1H), 7.54-7.28 (m, 1H), 7.28-7.11 (m, 1H), 7.11-7.04 (m, 1H), 4.32 (q, J=7.9, 7.5 Hz, 2H), 1.32 (t, J=7.1 Hz, 3H).

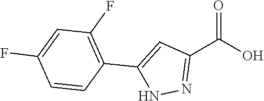 [SMILES: O=C(O)c1cc(-c2ccc(F)cc2F)[nH]n1]

### 5-(2,4-difluorophenyl)-1H-pyrazole-3-carboxylic acid (57)

To a solution of ester 56 (350 mg, 1.388 mmol, 1.0 eq.) in MeOH (2.0 mL) was added LiOH·H₂O (2.0 M, 1.0 mL, 1.5 eq.). The mixture was stirred at RT for 2 h. The volatiles were removed by rotary evaporation. The resultant mixture was adjusted to pH=1 with aq. 1.0 M HCl. The precipitate was collected by filtration and dried to give the title compound a white solid (300 mg, 1.34 mmol, 96%). LCMS (acidic mode) Rₜ 3.43 min, purity 95% [M+H]⁺ calculated 225.05, found 224.95. ¹H NMR (500 MHz, DMSO-d₆) δ 13.85 (s, 1H), 8.03-7.91 (m, 1H), 7.42-7.31 (m, 1H), 7.25-7.14 (m, 1H), 7.00-6.93 (m, 1H).

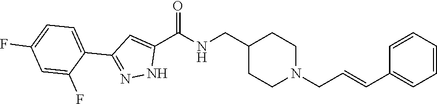 [SMILES: O=C(NCC1CCN(C/C=C/c2ccccc2)CC1)c1cc(-c2ccc(F)cc2F)n[nH]1]

### 3-(2,4-difluorophenyl)-N-({1-[(2E)-3-phenylprop-2-en-1-yl]piperidin-4-yl}methyl)-1H-pyrazole-5-carboxamide hydrochloric acid salt (58)

The title compound was synthesized according to general procedure E. For the first step, acid 57 (0.11 g, 0.50 mmol, 1.0 eq.), HATU (0.29 g 0.75 mmol, 1.5 eq.), DMF (2.5 mL), DIPEA (78 mg, 0.60 mmol, 1.2 eq.) and tert-butyl 4-(aminomethyl)piperidine-1-carboxylate (0.13 g, 0.60 mmol, 1.2 eq.) were used. For the second step, dioxane (2.0 mL) and MeOH (3.0 mL) and 4M HCl in dioxane (1.3 mL, 5.0 mmol, 10 eq.) were used. For the third step, the previous intermediate (0.13 g, 0.42 mmol, 1.0 eq.), THE (5.0 mL), TEA (58 μL, 0.42 mmol, 1.0 eq.), cinnamaldehyde (63 μL, 0.50 mmol, 1.2 eq.), STAB-H (0.22 g, 1.1 mmol, 2.5 eq.) were used. In addition to the reverse phase column purification described in general procedure E, a preparative column purification (using a linear gradient of 100% water to 100% ACN, dissolved in DMSO) was also needed, which afforded a light brown solid (6 mg, 0.01 mmol, 3%). LCMS (acidic mode) Rₜ 3.19 min, purity >99%, [M+H]⁺ calculated 437.21, found 437.25. ¹H NMR (600 MHz, Methanol-d4) δ 7.82 (s, 1H), 7.52-7.48 (m, 2H), 7.38-7.35 (m, 2H), 7.34-7.30 (m, 1H), 7.17-7.04 (m, 3H), 6.91 (d, J=15.7 Hz, 1H), 6.33 (dt, J=15.3, 7.4, 7.4 Hz, 1H), 3.90 (d, J=7.4 Hz, 2H), 3.67-3.60 (m, 2H), 3.35 (d, J=6.7 Hz, 2H), 3.05-2.96 (m, 2H), 2.11-2.03 (m, 2H), 2.03-1.95 (m, 1H), 1.64-1.50 (m, 2H). ¹³C NMR (151 MHz, Methanol-d4) δ 162.9 (dd, J=250.0, 12.4 Hz), 162.9, 159.5 (dd, J=251.8, 11.8 Hz), 140.5, 135.3, 129.2 (dd, J=9.5, 4.4 Hz), 128.7, 128.5, 126.7, 116.1, 111.7 (d, J=21.6 Hz), 104.7 (d, J=5.6 Hz), 104.2 (dd, J=26.1, 26.1 Hz), 58.6, 51.9, 43.2, 34.2, 27.3. HRMS m/z [M+H]⁺ C₂₅H₂₇F₂N₄O⁺ calculated 437.2148, found 437.2158.

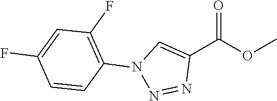 [SMILES: COC(=O)c1cn(-c2ccc(F)cc2F)nn1]

### methyl 1-(2,4-difluorophenyl)-1H-1,2,3-triazole-4-carboxylate (59)

To an ice-cold solution of 2,4-difluoroaniline (1.00 g, 7.75 mmol, 1.0 eq.) IACN (40 mL) was added t-BuONO (1.40 mL, 11.6 mmol, 1.5 eq.) followed by dropwise addition of TMSN3 (1.20 mL, 9.29 mmol, 1.2 eq.). The resulting mixture was stirred for 2 h in the ice bath and then warmed to rt. Methyl propiolate (785 μL, 7.75 mmol, 1.0 eq.), CuSO4.5H₂O (97.0 mg, 0.389 mmol, 0.05 eq.) and sodium ascorbate (230 mg, 1.16 mmol mmol, 0.15 eq.) were added. The mixture was stirred at RT for 16 h. The volatiles were removed in vacuo. Precipitation by MeOH gave the title compound as a brown solid (1.20 g, 5.02 mmol, 65%). LCMS (acidic mode) Rₜ 3.73 min, purity 63% [M+H]⁺ calculated 240.05, found 240.00. ¹H NMR (500 MHz, DMSO-d₆) δ 9.28 (d, J=1.5 Hz, 1H), 7.95 (ddd, J=8.9, 8.9, 5.8 Hz, 1H), 7.73 (ddd, J=10.8, 9.0, 2.7 Hz, 1H), 7.44-7.34 (m, 1H), 3.89 (s, 3H).

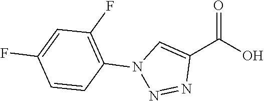 [SMILES: O=C(O)c1cn(-c2ccc(F)cc2F)nn1]

### 1-(2,4-difluorophenyl)-1H-1,2,3-triazole-4-carboxylic acid (60)

NaOH (2.0 M, 2.0 mL) was added to a solution of ester 59 (0.14 mg, 0.60 mmol, 1.0 eq.) in MeOH (2.0 mL) at RT. The mixture was stirred for 2 h and the MeOH was removed by rotary evaporation. The resultant mixture was adjusted to pH=1 with 1.0 M HCl solution. The precipitate was collected by filtration and dried to give the product as a brown powder (82 mg, 0.36 mmol, 61%). LCMS (acidic mode) Rₜ 3.00 min, purity >92% [M+H]⁺ calculated 226.09, not detected. ¹H NMR (500 MHz, Methanol-d₄) δ 8.89 (d, J=2.1 Hz, 1H), 7.92 (ddd, J=8.8, 5.6 Hz, 1H), 7.38 (ddd, J=11.0, 8.5, 2.7 Hz, 1H), 7.25 (dddd, J=9.1, 7.8, 2.7, 1.6 Hz, 2H).

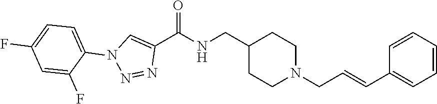 [SMILES: O=C(NCC1CCN(C/C=C/c2ccccc2)CC1)c1cn(-c2ccc(F)cc2F)nn1]

### 1-(2,4-difluorophenyl)-N-({1-[(2E)-3-phenylprop-2-en-1-yl]piperidin-4-yl}methyl)-1H-1,2,3-triazole-4-carboxamide hydrochloric acid salt (61)

The title compound was synthesized according to general procedure E. For the first step, acid 60 (74 mg, 0.33 mmol, 1.0 eq.), HATU (0.16 g 0.43 mmol, 1.3 eq.), DMF (2.5 mL), DIPEA (51 mg, 0.39 mmol, 1.2 eq.) and tert-butyl 4-(aminomethyl)piperidine-1-carboxylate (85 mg, 0.39 mmol, 1.2 eq.) were used. For the second step, dioxane (2.0 mL), MeOH (3.0 mL) and 4M HCl in dioxane (0.80 mL, 3.3 mmol, 10 eq.) were used. For the third step, the previous intermediate (0.11 g, 0.33 mmol, 1.0 eq.), THE (4.0 mL), TEA (46 μL, 0.33 mmol, 1.0 eq.), cinnamaldehyde (50 μL, 0.40 mmol, 1.2 eq.), STAB-H (0.17 g, 0.82 mmol, 2.5 eq.) were used. The product was obtained as a pale-yellow solid (73 mg, 0.15 mmol, 47%). LCMS (acidic mode) Rₜ 3.19 min, purity >99%, [M+H]⁺ calculated 438.21, found 438.25. ¹H NMR (600 MHz, Methanol-d4) δ 8.76 (d, J=1.8 Hz, 1H), 7.98-7.80 (m, 1H), 7.54-7.48 (m, 2H), 7.42-7.28 (m, 5H), 7.27-7.22 (m, 1H), 6.91 (d, J=15.6 Hz, 1H), 6.34 (dt, J=14.6, 6.8 Hz, 1H), 3.90 (d, J=7.2 Hz, 2H), 3.70-3.58 (m, 2H), 3.44-3.37 (m, 2H), 3.08-2.95 (m, 2H), 2.14-2.05 (m, 2H), 2.04-1.99 (m, 1H), 1.64-1.54 (m, 2H). ¹³C NMR (151 MHz, Methanol-d4) δ 164.6 (dd, J=251.9, 11.3 Hz), 162.4, 156.3 (dd, J=254.0, 12.9 Hz), 144.3, 142.0, 141.8, 136.6, 130.1, 129.8, 128.7 (d, J=4.9 Hz), 128.5 (d, J=10.3 Hz), 128.1, 122.8 (dd, J=11.0, 3.6 Hz), 117.5, 113.7 (dd, J=23.2, 3.8 Hz), 106.5 (dd, J=27.5, 23.8 Hz), 60.1, 53.3, 44.7, 35.6, 28.7. HRMS m/z [M+H]⁺ C₂₄H₂₆F₂N₅O⁺ calculated 438.1200, found 438.2117.

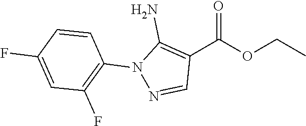 [SMILES: CCOC(=O)c1cnn(-c2ccc(F)cc2F)c1N]

### Ethyl 5-amino-1-(2,4-difluorophenyl)-1H-pyrazole-4-carboxylate (62)

Ethyl-2-cyano-3-ethoxyacrylate (468 mg, 2.77 mmol, 1.0 equiv.), DIPEA (0.5 mL, 3 mmol, 1.1 equiv.) and 2,4-difluorohydrazine hydrochloride (550 mg, 3.05 mmol, 1.1 equiv.) were suspended in EtOH (4 mL). The mixture was stirred at reflux for 16 h. The mixture was concentrated to obtain the title compound as a yellow solid (487 mg, 1.83 mmol, 66%). LCMS (acidic mode) Rₜ 3.85 min, purity 92% [M+H]⁺ calculated 268.09, found 268.04. ¹H NMR (500 MHz, DMSO-d₆) δ 7.69 (s, 1H), 7.62-7.48 (m, 2H), 7.30-7.22 (m, 1H), 6.39 (s, 2H), 4.21 (q, J=7.0 Hz, 2H), 1.26 (t, J=7.1 Hz, 3H).

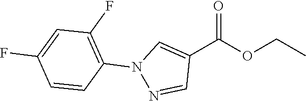 [SMILES: CCOC(=O)c1cnn(-c2ccc(F)cc2F)c1]

### Ethyl 1-(2,4-difluorophenyl)-1H-pyrazole-4-carboxylate (63)

A solution of tBuONO₂ (0.85 mL, 6.42 mmol, 6 eq.) in THE (2 mL) was stirred at reflux for 20 min. Amine 62 (285 mg, 1.07 mmol, 1.0 eq.) was added and the mixture was stirred at reflux for 2 h. The volatiles were evaporated in vacuo to obtain the title compound as an orange solid (266 mg, 1.06 mmol, 99%). LCMS (acidic mode) Rₜ 4.54 min, purity >97% [M+H]⁺ calculated 253.08, found 252.95. ¹H NMR (500 MHz, Chloroform-d) δ 8.40 (d, J=2.6 Hz, 1H), 8.11 (s, 1H), 7.91-7.81 (m, 1H), 7.08-6.98 (m, 2H), 4.34 (q, J=7.2 Hz, 2H), 1.37 (t, J=7.1 Hz, 3H).

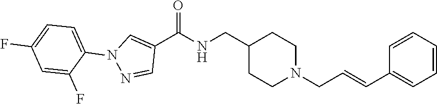 [SMILES: O=C(NCC1CCN(C/C=C/c2ccccc2)CC1)c1cnn(-c2ccc(F)cc2F)c1]

### N-((1-cinnamylpiperidin-4-yl)methyl)-1-(2,4-difluorophenyl)-1H-pyrazole-4-carboxamide (64)

The title compound was synthesised according to general procedure F, but required ester hydrolysis first. Ester 63 (266 mg, 0.896 mmol, 1.0 eq.) was dissolved in THE (5 mL). Aq. 2M NaOH (1.3 mL, 2.7 mmol, 3 eq.) was added. The mixture was stirred at reflux for 16 h. The mixture was acidified to pH=5 with 2 M aq. HCl solution and extracted with DCM (3×). The combined organic phases were dried over anhydrous Na₂SO₄, filtered, and concentrated to obtain 1-(2,4-difluorophenyl)-1H-pyrazole-4-carboxylic acid as a white solid. This acid (198 mg, 0.892 mmol, 1.0 eq.) was reacted according to general procedure F using (1-cinnamylpiperidin-4-yl)methanamine hydrochloride (271 mg, 0.892 mmol, 1.0 eq.), (COCl)₂ (0.09 mL, 1 mmol, 1.2 eq.), and TEA (0.25 mL, 1.8 mmol, 2.0 eq.). The mixture was stirred for 3 h at RT. The crude product was purified by RP column chromatography (using a linear gradient of 0-75% ACN in water with both 0.1% FA) to obtain the title compound as a white solid (60 mg, 15%). LCMS (acidic mode) Rₜ 3.29 min, purity >96% [M+H]⁺ calculated 437.21, found 437.25. ¹H NMR (500 MHz, DMSO-d₆) δ 8.62 (d, J=2.2 Hz, 1H), 8.27 (t, J=5.8 Hz, 1H), 8.16 (s, 1H), 7.85 (ddd, J=9.0, 9.0, 5.9 Hz, 1H), 7.60 (ddd, J=11.6, 8.9, 2.8 Hz, 1H), 7.45-7.40 (m, 2H), 7.34-7.25 (m, 3H), 7.25-7.19 (m, 1H), 6.50 (d, J=15.9 Hz, 1H), 6.29 (dt, J=16.0, 6.5 Hz, 1H), 3.12 (dd, J=6.3, 6.3 Hz, 2H), 3.09-3.03 (m, 2H), 2.92-2.82 (m, 2H), 1.94-1.84 (m, 2H), 1.70-1.61 (m, 2H), 1.56-1.44 (m, 1H), 1.27-1.13 (m, 2H). ¹³C NMR (126 MHz, DMSO-d₆) b 161.1, 160.7 (dd, J=235.0, 11.2 Hz), 153.8 (dd, J=251.7, 13.1 Hz), 140.5, 136.7, 132.3 (d, J=6.6 Hz), 131.6, 128.6, 127.6, 127.3, 126.5 (d, J=10.1 Hz), 126.2, 124.4 (d, J=4.0 Hz), 120.5, 112.5 (dd, J=22.7, 3.6 Hz), 105.6 (dd, J=27.2, 24.4 Hz), 60.6, 53.1, 44.2, 35.9, 29.9. HRMS m/z [M+H]⁺ C₂₅H₂₇F₂N₄O⁺ calculated 437.2147, found 437.2148.

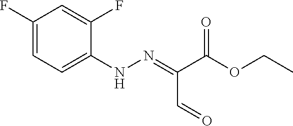 [SMILES: CCOC(=O)/C(C=O)=N/Nc1ccc(F)cc1F]

### Ethyl 2-(2-(2,4-difluorophenyl)hydrazineylidene)-3-oxopropanoate (65)

To an ice-cold solution of 2,4-difluoroaniline (1.0 g, 7.9 mmol, 1.0 eq.) in water (10 mL) and concentrated aq. HCl (2.0 mL, 23 mmol, 3.0 eq.) was dropwise added aq. NaNO₂ (1.6 M, 4.9 mL, 7.9 mmol, 1.0 eq.). After stirring for 10 min, a solution of ethyl-(2E)-3-(dimethylamino)acrylate (1.24 g, 8.66 mmol, 1.0 eq.) and KOAc (1.16 g, 11.8 mmol) in EtOH (15 mL) was added dropwise. The resulting mixture was warmed to RT and stirred for 10 min. The resulting mixture was diluted with EtOAc and washed with water (2×) and brine. The organic layer was dried over anhydrous Na₂SO₄, filtered, and concentrated to obtain the title compound as a red solid (2.20 g, 6.72 mmol, 85%). LCMS (acidic mode) Rₜ 4.63 min, purity 78%, [M+H]⁺ calculated 257.07, found 257.00. ¹H NMR (500 MHz, DMSO-d₆) δ 14.47 (s, 1H), 9.84 (s, 1H), 7.80-7.70 (m, 1H), 7.55-7.46 (m, 1H), 7.27-7.17 (m, 1H), 4.42-4.23 (m, 2H), 1.31 (t, J=7.1 Hz, 3H). Z/E-isomers were observed, and the major product peaks are reported.

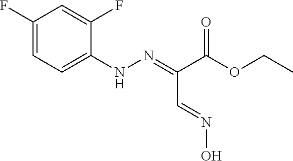 [SMILES: CCOC(=O)C(/C=N/O)=N/Nc1ccc(F)cc1F]

### Ethyl 2-(2-(2,4-difluorophenyl)hydrazineylidene)-59ydroxyaminoino)propanoate (66)

Aldehyde 65 (2.01 g, 6.13 mmol, 1.0 eq.) was dissolved in EtOH (20 mL). NH₂OH·HCl (0.51 g, 7.36 mmol, 1.2 eq.) and KOAc (1.50 g, 15.3 mmol, 2.5 eq.) were added. The mixture was stirred for 16 h at RT. The mixture was diluted with EtOAc and washed twice with water and once with brine. The organic layer was dried over anhydrous Na₂SO₄, filtered and concentrated in vacuo. The crude product was purified by NP column chromatography (using a linear gradient of 5-40% EtOAc in cHex) to obtain the title compound as a dark orange/red solid (0.775 g, 2.88 mmol, 47%). LCMS (acidic mode) Rₜ 4.00 min, purity 74%, [M+H]⁺ calculated 272.08, found 272.05. ¹H NMR (500 MHz, DMSO-d₆) δ 12.66 (d, J=2.0 Hz, 1H), 12.27 (s, 1H), 8.30 (s, 1H), 7.65 (ddd, J=9.2, 9.2, 5.8 Hz, 1H), 7.42 (ddd, J=11.6, 8.7, 2.7 Hz, 1H), 7.24-7.15 (m, 1H), 4.25 (q, J=7.1 Hz, 2H), 1.29 (t, J=7.1 Hz, 3H).

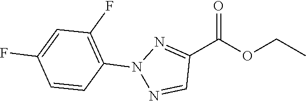 [SMILES: CCOC(=O)c1cnn(-c2ccc(F)cc2F)n1]

### Ethyl 2-(2,4-difluorophenyl)-2H-1,2,3-triazole-4-carboxylate (67)

Oxime 66 (765 mg, 2.82 mmol) was stirred for 1 h in Ac₂O (10 mL) at 60° C. Water was added to the mixture and the aqueous layer was extracted with EtOAc. The organic layer was dried over anhydrous Na₂SO₄, filtered and concentrated in vacuo to obtain the title compound as a yellow solid (0.691, 97%). LCMS (acidic mode) Rₜ 4.43 min, [M+H]⁺ calculated 254.07 found 254.00. ¹H NMR (500 MHz, DMSO-d₆) δ 8.64 (s, 1H), 7.96 (ddd, J=8.8, 8.8, 5.9 Hz, 1H), 7.68 (ddd, J=11.4, 9.0, 2.7 Hz, 1H), 7.35 (dddd, J=9.2, 7.9, 2.8, 1.4 Hz, 1H), 4.38 (q, J=7.1 Hz, 2H), 1.33 (t, J=7.1 Hz, 3H).

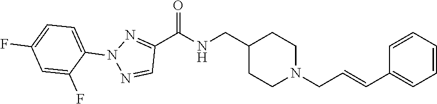 [SMILES: O=C(NCC1CCN(C/C=C/c2ccccc2)CC1)c1cnn(-c2ccc(F)cc2F)n1]

### N-((1-cinnamylpiperidin-4-yl)methyl)-2-(2,4-difluorophenyl)-2H-1,2,3-triazole-4-carboxamide (68)

The title compound was synthesised according to general procedure G. (1-cinnamylpiperidin-4-yl)methanamine hydrochloride (640 mg, 2.11 mmol, 1.0 eq.), ester 67 (691 mg, 2.32 mmol, 1.0 eq.), 3-chlorophenol (298 mg, 2.32 mmol, 1.1 eq.) and K₂CO₃ (698 mg, 5.05 mmol, 2.5 eq.) were used. The mixture was heated at reflux for 36 h, after which additional 3-chlorophenol (298 mg, 2.32 mmol, 1.1 eq.) was added and the mixture was stirred for another 24 h at reflux. The crude product was first purified by NP column chromatography (using a linear gradient of 20-40% EtOAc:MeOH:TEA 90:5:5 in cHex) and afterwards RP column chromatography (using a linear gradient of 0-100% ACN in water with both 0.1% FA) to obtain the title compound as a light yellow solid (199 mg, 22%). LCMS (acidic mode) Rₜ 3.43 min, purity >99%, [M+H]⁺ calculated: 438.21 found: 438.35. ¹H NMR (500 MHz, DMSO-d₆) δ 8.70 (t, J=6.0 Hz, 1H), 8.48 (s, 1H), 7.96 (ddd, J=8.8, 8.8, 5.8 Hz, 1H), 7.68 (ddd, J=11.4, 9.0, 2.8 Hz, 1H), 7.42 (d, J=7.3 Hz, 2H), 7.39-7.34 (m, 1H), 7.31 (t, J=7.6 Hz, 2H), 7.22 (t, J=7.4 Hz, 1H), 6.50 (d, J=15.9 Hz, 1H), 6.29 (dt, J=16.0, 6.5 Hz, 1H), 3.17 (dd, J=6.5, 6.5 Hz, 2H), 3.06 (d, J=6.6 Hz, 2H), 2.91-2.81 (m, 2H), 1.92-1.83 (m, 2H), 1.69-1.60 (m, 2H), 1.60-1.50 (m, 1H), 1.26-1.12 (m, 2H). ¹³C NMR (126 MHz, DMSO-d₆) δ 162.1 (dd, J=250.0, 11.4 Hz), 158.9, 154.8 (dd, J=255.3, 14.1 Hz), 144.8, 136.7, 136.6, 131.6, 128.6, 127.8 (d, J=10.5 Hz), 127.7, 127.3, 126.2, 124.3 (dd, J=10.1, 4.0 Hz), 112.7 (dd, J=23.1, 4.0 Hz), 106.0 (dd, J=27.4, 23.7 Hz), 60.6, 53.0, 44.3, 35.7, 29.8. HRMS m/z [M+H]⁺ C₂₄H₂₆F₂N₅O⁺ calculated 438.2100, found 438.2100.

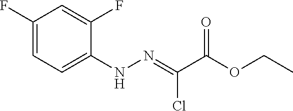 [SMILES: CCOC(=O)/C(Cl)=N/Nc1ccc(F)cc1F]

### Ethyl 2-chloro-2-(2-(2,4-difluorophenyl)hydrazineylidene)acetate (69)

2,4-Difluorophenylaniline (1.50 g, 11.6 mmol, 1.0 eq.) was added to a solution of conc. aq. HCl (3.9 mL, 46 mmol, 4.0 eq.), EtOH (5.8 mL), and water (2.3 mL). A solution of NaNO₂ (1.60 g, 23.2 mmol, 2.0 eq.) in water (12 mL) was added dropwise to the mixture at −5° C. The mixture was stirred for 30 min at −5° C. A cold solution of ethyl 2-chloro-3-oxobutanoate (1.91 g, 11.6 mmol, 1.0 eq.) and NaOAc (2.86 g, 34.9 mmol, 3.0 eq.) in EtOH (6 mL) and water (5 mL) was added. The mixture was stirred at −5° C. for 4 h. The mixture was quenched with water (100 mL) and subsequently stirred for 16 h. The solid was collected by filtration and dried in a vacuum oven to obtain the title compound as a yellow solid (2.76 g, 10.4 mmol 90% yield), which was used in the next step without further purification. LCMS (acidic mode) Rₜ 4.84 min, purity 85% [M+H]⁺ calculated 263.04, found 263.00. ¹H NMR (500 MHz, DMSO-d₆) δ 9.86 (s, 1H), 7.44 (ddd, J=9.2, 9.2, 5.9 Hz, 1H), 7.36 (ddd, J=11.6, 8.9, 2.8 Hz, 1H), 7.16-7.07 (m, 1H), 4.29 (q, J=7.1 Hz, 2H), 1.28 (t, J=7.1 Hz, 3H).

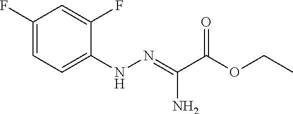 [SMILES: CCOC(=O)/C(N)=N/Nc1ccc(F)cc1F]

### Ethyl 2-amino-2-(2-(2,4-difluorophenyl)hydrazineylidene)acetate (70)

Chloride 69 (1.4 g, 5.3 mmol, 1.0 eq.) was dissolved in dioxane (20 mL). Subsequently, NH₃ in MeOH (7M, 3.0 mL, 21 mmol, 4.0 eq.) was added to the reaction. The mixture was stirred at RT for 16 h. The solution was concentrated. The residue was dissolved in EtOAc and washed with brine. The organic layer was dried with anhydrous Na₂SO₄, filtered and concentrated to obtain the title compound as a dark yellow solid (1.25 g, 5.09 mmol, 96%). LCMS (acidic mode) Rₜ 3.42 min, purity 89% [M+H]⁺ calculated 244.09, found 244.00. ¹H NMR (500 MHz, DMSO-d₆) δ 8.27 (s, 1H), 7.39 (ddd, J=9.3, 9.3, 6.0 Hz, 1H), 7.18 (ddd, J=11.9, 8.9, 2.9 Hz, 1H), 7.00 (dddd, J=8.6, 8.6, 2.9, 1.4 Hz, 1H), 6.29 (s, 2H), 4.24 (q, J=7.1 Hz, 2H), 1.29 (t, J=7.1 Hz, 3H).

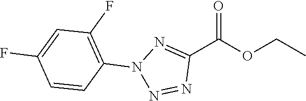 [SMILES: CCOC(=O)c1nnn(-c2ccc(F)cc2F)n1]

### Ethyl 2-(2,4-difluorophenyl)-2H-tetrazole-5-carboxylate (71)

To a solution of compound 70 (1.01 g, 4.17 mmol, 1.0 eq.) in THE (20 mL) was added AcOH (0.72 mL, 12 mmol, 3.0 eq.). The mixture was heated to 85° C. Aq. NaNO₂ (2.6N, 1.9 mL, 5.0 mmol, 1.2 eq.) was added and the mixture was stirred at 85° C. for 4 h. The mixture was concentrated in vacuo. The residue was dissolved in EtOAc and washed with satd. aq. NaHCO₃. The aqueous layer was extracted with EtOAc. The combined organic layers were washed with brine, dried over anhydrous Na₂SO₄, filtered, and concentrated in vacuo to obtain the title compound as a yellow solid (730 mg, 2.88 mmol, 69%). LCMS (acidic mode) Rₜ 4.22 min, purity >99% [M+H]⁺ calculated 255.07, found 254.85. ¹H NMR (500 MHz, DMSO-d₆) δ 8.12 (ddd, J=8.8, 8.7, 5.8 Hz, 1H), 7.83 (ddd, J=11.0, 9.0, 2.7 Hz, 1H), 7.47 (dddd, J=9.2, 8.0, 2.8, 1.5 Hz, 1H), 4.50 (q, J=7.1 Hz, 2H), 1.39 (t, J=7.1 Hz, 3H).

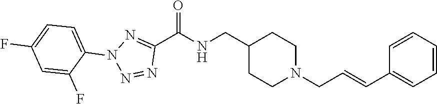 [SMILES: O=C(NCC1CCN(C/C=C/c2ccccc2)CC1)c1nnn(-c2ccc(F)cc2F)n1]

### N-((1-cinnamylpiperidin-4-yl)methyl)-2-(2,4-difluorophenyl)-2H-tetrazole-5-carboxamide (72)

The title compound w61ydroxyaminsed according to general procedure G. (1-cinnamylpiperidin-4-yl)methanamine hydrochloride (735 mg, 2.42 mmol, 1.2 eq.), ester 71 (604 mg, 2.02 mmol, 1.0 eq.), 3-chlorophenol (260 mg, 2.02 mmol, 1.0 eq.) and K₂CO₃ (698 mg, 5.05 mmol, 2.5 eq.) were used. The mixture was heated at reflux for 16 h. The residue was first purified by NP column chromatography (using a linear gradient of 20-40% EtOAc:MeOH:TEA 90:5:5 in cHex) and afterwards by RP column chromatography (using a linear gradient of 0-70% ACN in water with both 0.1% FA) to obtain the title compound as a yellow solid (205 mg, 23%). LCMS (acidic mode) Rₜ 3.20 min, purity >97%, [M+H]⁺ calculated: 439.21 found: 439.25. ¹H NMR (500 MHz, DMSO-d₆) δ 9.24 (t, J=6.0 Hz, 1H), 8.09 (ddd, J=8.7, 8.7, 5.8 Hz, 1H), 7.80 (ddd, J=11.4, 8.9, 2.7 Hz, 1H), 7.48-7.40 (m, 3H), 7.31 (t, J=7.6 Hz, 2H), 7.25-7.19 (m, 1H), 6.51 (d, J=15.9 Hz, 1H), 6.29 (dt, J=15.9, 6.5 Hz, 1H), 3.22 (dd, J=6.4, 6.4 Hz, 2H), 3.08 (d, J=6.6 Hz, 2H), 2.92-2.84 (m, 2H), 1.96-1.84 (m, 2H), 1.71-1.55 (m, 3H), 1.26-1.15 (m, 2H). ¹³C NMR (126 MHz, DMSO-d₆) δ 163.3 (dd, J=252.3, 11.4 Hz), 160.1, 156.0, 154.9 (dd, J=257.5, 13.6 Hz), 136.7, 131.8, 128.6, 128.3 (d, J=10.9 Hz), 127.4, 127.3, 126.2, 121.0 (dd, J=11.0, 4.0 Hz), 113.1 (dd, J=23.2, 3.7 Hz), 106.3 (dd, J=27.7, 23.4 Hz), 60.5, 52.9, 44.5, 35.5, 29.7. HRMS m/z [M+H]⁺ C₂₃H₂₅F₂N₆O⁺ calculated 439.2052, found 439.2039.

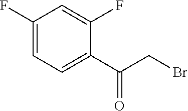 [SMILES: O=C(CBr)c1ccc(F)cc1F]

### 2-bromo-1-(2,4-difluorophenyl)ethan-1-one (73)

To a solution of 1-(2,4-difluorophenyl)ethan-1-one (5.00 g, 32.0 mmol, 1.0 eq.) in DCM (15 mL) was added NBS (6.27 g, 35.2 mmol, 1.1 eq.) and pTsOH (8.27 g, 48.0 mmol, 1.5 eq.). The mixture was reacted in the microwave at 40° C. for 15 min. The mixture was extracted with water. The organic layer was dried over anhydrous Na₂SO₄, filtered, and the solvent was removed in vacuo. The resulting crude material was purified using NP column chromatography (using a linear gradient of 0-100% EtOAc in cHex). The title compound was obtained as a crystalline solid (7.1 g, 30.2 mmol, 94%). LCMS (acidic mode) Rₜ 4.37 min, purity 86% [M+H]⁺ calculated 234.95, not detected. ¹H NMR (500 MHz, DMSO-d₆) δ 8.01 (ddd, J=8.7, 6.6 Hz, 1H), 7.47 (ddd, J=11.7, 9.3, 2.6 Hz, 1H), 7.30-7.25 (m, 1H), 4.82 (d, J=2.3 Hz, 2H).

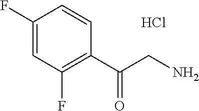 [SMILES: NCC(=O)c1ccc(F)cc1F ; Cl]

### 2-amino-1-(2,4-difluorophenyl)ethan-1-one hydrochloride (74.HCl)

To a solution of bromide 73 (1.50 g, 6.38 mmol, 1.0 eq.) in CHCl₃ (20 mL) was added hexamethylenetetramine (1.34 g, 9.57 mmol, 1.5 eq.). The mixture was stirred for 12 h at RT. The solids were filtered, washed with CHCl₃ and dissolved in EtOH (10 mL). Concentrated aq. HCl (0.5 mL) was added and the mixture was heated at reflux for 3 h. After cooling to RT, the precipitate was filtered, washed with EtOH and dried in vacuo to obtain the title compound as a white solid (1.20 g, 5.78 mmol, 91%). LCMS (acidic mode) Rₜ 1.62 min, purity 92% [M+H]1 calculated 172.06, found 172.00. ¹H NMR (500 MHz, DMSO-d₆) δ 8.52 (s, 2H), 8.07 (ddd, J=8.7, 6.6 Hz, 1H), 7.54 (ddd, J=11.7, 9.3, 2.5 Hz, 1H), 7.35-7.29 (m, 2H), 4.40 (s, 2H).

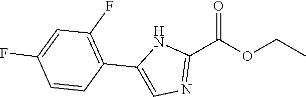 [SMILES: CCOC(=O)c1ncc(-c2ccc(F)cc2F)[nH]1]

### Ethyl 4-(2,4-difluorophenyl)-1H-imidazole-2-carboxylate (75)

To an ice bath cooled solution of ethyl thiooxamate (2.71 g, 20.4 mmol, 1.0 eq.) in DCM was slowly added Me₃O⁺BF₄⁻ (3.31 g, 22.4 mmol, 1.1 eq.). The mixture was stirred for 30 min. The ice bath was removed and the mixture was stirred for an additional h. The volatiles were removed in vacuo. The obtained ethyl 2-imino-2-(methylthio)acetate was added in dioxane (15 mL) to a solution of NaOAc (5.94 g, 72.3 mmol, 5.0 eq.), 74.HCl (3.00 g, 14.5 mmol, 1.3 eq.) in CH₃COOH (5 mL). The mixture was heated at 65° C. for 3 h. The mixture was cooled to RT. Satd. aq. NaHCO₃ was slowly added to quench the reaction. The precipitate was mixed with H₂O and EtOAc. The organic layer was washed with H₂O and brine, dried over anhydrous Na₂SO₄, filtered, and concentrated in vacuo. The crude was purified by NP column chromatography (using a linear gradient of 5-25% EtOAc in cHex) to obtain the title compound of colorless crystals (3.00 g, 11.9 mmol, 82%). LCMS (acidic mode) Rₜ 3.90 min, purity >99.9% [M+H]⁺ calculated 253.08, found 253.00. ¹H NMR (500 MHz, DMSO-d₆) δ 13.63 (s, 1H), 8.07 (ddd, J=8.9, 6.7 Hz, 1H), 7.61 (d, J=4.0 Hz, 1H), 7.32 (ddd, J=11.7, 9.2, 2.6 Hz, 1H), 7.19-7.12 (m, 1H), 4.35 (q, J=7.2 Hz, 2H), 1.34 (t, J=7.1 Hz, 3H).

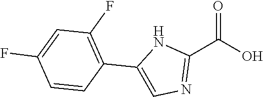 [SMILES: O=C(O)c1ncc(-c2ccc(F)cc2F)[nH]1]

### 4-(2,4-difluorophenyl)-1H-imidazole-2-carboxylic acid (76)

To a solution of ester 75 (350 mg, 1.39 mmol, 1.0 eq.) in MeOH (2.0 mL) was added LiOH·H₂O (2.0 M, 1.0 mL, 1.5 eq.). The mixture was stirred at RT for 2 h. The volatiles were removed by rotary evaporation. The resultant mixture was adjusted to pH=5 with 1.0 M HCl solution. The precipitate was collected by filtration and dried to give the title compound a white solid (300 mg, 1.34 mmol, 96%). LCMS (acidic mode) Rₜ 2.58 min, purity >99% [M+H]⁺ calculated 225.05, found 224.95. ¹H NMR (500 MHz, DMSO-d₆) δ 8.06 (ddd, J=8.9, 6.7 Hz, 1H), 7.61 (d, J=3.8 Hz, 1H), 7.34 (ddd, J=11.7, 9.3, 2.6 Hz, 1H), 7.18 (ddd, J=8.6, 2.9 Hz, 1H).

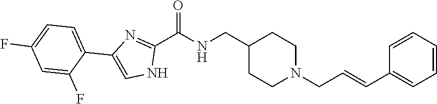 [SMILES: O=C(NCC1CCN(C/C=C/c2ccccc2)CC1)c1nc(-c2ccc(F)cc2F)c[nH]1]

### 4-(2,4-difluorophenyl)-N-({1-[(2E)-3-phenylprop-2-en-1-yl]piperidin-4-yl}methyl)-1H-imidazole-2-carboxamide hydrochloric acid salt (77)

The title compound was synthesized according to general procedure E. For the first step, acid 76 (0.11 g, 0.50 mmol, 1.0 eq.), HATU (0.29 g, 0.75 mmol, 1.5 eq.), DMF (2.5 mL), DIPEA (78 mg, 0.60 mmol, 1.2 eq.) and tert-butyl 4-(aminomethyl)piperidine-1-carboxylate (0.13 g, 0.60 mmol, 1.2 eq.) were used. For the second step, dioxane (2.0 mL), MeOH (3.0 mL) and 4M HCl in dioxane (1.3 mL, 5.0 mmol, 10 eq.) were used. For the third step, the previous intermediate (0.14 g, 0.42 mmol, 1.0 eq.), THE (5.0 mL), TEA (60 μL, 0.42 mmol, 1.0 eq.), cinnamaldehyde (65 μL, 0.51 mmol, 1.2 eq.) and STAB-H (0.22 g, 1.1 mmol, 2.5 eq.) were used to obtain a transparent amorphous solid (0.11 g, 0.23 mmol, 54%). LCMS (acidic mode) Rₜ 3.35 min, purity >96%, [M+H]⁺ calculated 437.21, found 437.25. ¹H NMR (600 MHz, Methanol-d₄) δ 8.12-8.04 (m, 1H), 7.88-7.79 (m, 1H), 7.53-7.49 (m, 2H), 7.42-7.27 (m, 4H), 7.21-7.12 (m, 2H), 6.92 (d, J=15.7 Hz, 1H), 6.36 (dt, J=15.3, 7.4 Hz, 1H), 3.91 (d, J=7.4 Hz, 2H), 3.69-3.62 (m, 2H), 3.46-3.40 (m, 2H), 3.07-2.98 (m, 2H), 2.19-2.08 (m, 2H), 2.08-2.00 (m, 1H), 1.67-1.56 (m, 2H). ¹³C NMR (151 MHz, Methanol-d₄) δ 163.3 (dd, J=251.0, 12.3 Hz), 159.9 (dd, J=252.1, 12.1 Hz), 155.2, 140.6, 138.9, 135.3, 130.6, 129.4 (dd, J=9.9, 4.1 Hz), 128.7, 128.5, 126.7, 119.1 (d, J=10.7 Hz), 116.1, 113.3 (dd, J=12.3, 2.5 Hz), 111.9 (dd, J=22.1, 3.4 Hz), 104.3 (dd, J=26.1, 26.1 Hz), 58.6, 51.8, 43.8, 33.9, 27.2. HRMS m/z [M+H]⁺ C₂₅H₂₆F₂N₃OS⁺ calculated 437.2148, found 437.2174.

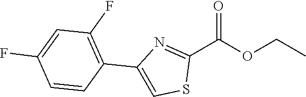 [SMILES: CCOC(=O)c1nc(-c2ccc(F)cc2F)cs1]

### Ethyl 4-(2,4-difluorophenyl)thiazole-2-carboxylate (78)

To a stirred solution of bromide 73 (536 mg, 2.28 mmol, 1.0 eq.) was added ethyl thioglycinate (395 mg, 2.96 mmol, 1.3 eq.) The mixture was heated at reflux for 12 h. The volatiles were removed in vacuo and purification by NP column chromatography (using a linear gradient of 5-20% EtOAc in cHex) gave the title compound as a yellow solid (512 mg, 1.90 mmol, 83%). LCMS (acidic mode) Rₜ 5.00 min, purity 96% [M+H]⁺ calculated 270.04, found 269.95. ¹H NMR (500 MHz, DMSO-d₆) b 8.33 (d, J=2.4 Hz, 1H), 8.13 (ddd, J=8.9, 6.7 Hz, 1H), 7.43 (ddd, J=11.7, 9.2, 2.5 Hz, 1H), 7.29-7.17 (m, 1H), 4.41 (q, J=7.1 Hz, 2H), 1.36 (t, J=7.1 Hz, 3H).

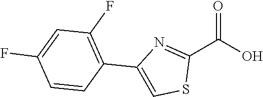 [SMILES: O=C(O)c1nc(-c2ccc(F)cc2F)cs1]

### 4-(2,4-difluorophenyl)thiazole-2-carboxylic acid (79)

To a solution of ester 78 (374 mg, 1.39 mmol, 1.0 eq.) in MeOH (2.0 mL) was added 2M aq. LiOH (1.0 mL, 1.5 eq.) at RT. The mixture was stirred for 2 h. The volatiles were removed by rotary evaporation. The resultant mixture was adjusted to pH=5 with 1.0 M HCl solution. The precipitate was collected by filtration and dried to give the title compound a white solid (310 mg, 1.29 mmol, 93%). LCMS (acidic mode) Rₜ 3.88 min, purity 97% [M+H]1 calculated 242.01, found no mass observed. ¹H NMR (500 MHz, DMSO-d₆) δ 8.29 (d, J=2.4 Hz, 1H), 8.14 (ddd, J=8.9, 8.9, 6.7 Hz, 1H), 7.43 (ddd, J=11.9, 9.3, 2.6 Hz, 1H), 7.25 (ddd, J=8.3, 8.3, 2.3 Hz, 1H).

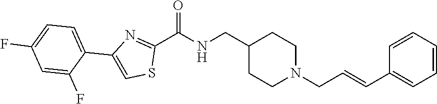 [SMILES: O=C(NCC1CCN(C/C=C/c2ccccc2)CC1)c1nc(-c2ccc(F)cc2F)cs1]

### 4-(2,4-difluorophenyl)-N-({1-[(2E)-3-phenylprop-2-en-1-yl]piperidin-4-yl}methyl)-1,3-thiazole-2-carboxamide (80)

The title compound was synthesized according to general procedure E. For the first step, acid 79 (0.12 g, 0.50 mmol, 1.0 eq.), HATU (0.29 g 0.75 mmol, 1.5 eq.), DMF (2.5 mL), DIPEA (78 mg, 0.60 mmol, 1.2 eq.) and tert-butyl 4-(aminomethyl)piperidine-1-carboxylate (0.13 g, 0.60 mmol, 1.2 eq.) were used. For the second step, dioxane (2.0 mL), MeOH (3.0 mL) and 4.0 M HCl in dioxane (1.3 mL, 5.0 mmol, 10 eq.) were used. For the third step, the previous intermediate (0.16 g, 0.46 mmol, 1.0 eq.), THE (5.0 mL), TEA (64 μL, 0.46 mmol, 1.0 eq.), cinnamaldehyde (70 μL, 0.55 mmol, 1.2 eq.) and STAB-H (0.24 g, 1.2 mmol, 2.5 eq.) were used. The title compound was obtained as a light pink solid (33 mg, 0.067 mmol, 15%). LCMS (acidic mode) Rₜ 3.60 min, purity >97%, [M+H]⁺ calculated 454.18, found 454.25. ¹H NMR (500 MHz, Chloroform-d) δ 8.29-8.10 (m, 1H), 7.85 (d, J=2.0 Hz, 1H), 7.80 (t, J=6.4, 6.4 Hz, 1H), 7.43-7.34 (m, 2H), 7.33-7.22 (m, 3H), 7.01-6.92 (m, 1H), 6.92-6.83 (m, 1H), 6.71 (d, J=15.5 Hz, 1H), 6.57-6.36 (m, 1H), 3.80-3.70 (m, 2H), 3.66-3.49 (m, 2H), 3.48-3.36 (m, 2H), 3.01-2.50 (m, 2H), 2.21-1.78 (m, 5H). ¹³C NMR (126 MHz, Chloroform-d) δ 162.7 (dd, J=251.3, 12.3 Hz), 162.1, 160.3 (dd, J=252.8, 11.8 Hz), 159.9, 149.3 (d, J=2.7 Hz), 140.1, 134.8, 131.1 (dd, J=9.6, 4.4 Hz), 129.2, 128.8, 127.1, 122.3 (d, J=14.1 Hz), 117.9 (dd, J=11.6, 3.8 Hz), 116.7, 112.0 (dd, J=21.2, 3.4 Hz), 104.5 (dd, J=25.9, 25.9 Hz), 59.4, 51.9, 44.5, 33.9, 26.9. HRMS m/z [M+H]⁺ C₂₅H₂₆F₂N₃OS⁺ calculated 454.1759, found 454.1781.

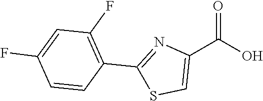 [SMILES: O=C(O)c1csc(-c2ccc(F)cc2F)n1]

### 2-(2,4-difluorophenyl)thiazole-4-carboxylic acid (81)

Ethyl 2-(2,4-difluorophenyl)thiazole-4-carboxylate (300 mg, 0.947 mmol, 1.0 eq.) was dissolved in THE (5 mL). Aq. NaOH (2M, 1.4 mL, 1.8 mmol, 3.0 eq.) was added. The mixture was stirred at RT for 3 h. The mixture was acidified to pH 5 with 2 M aq. HCl solution and extracted with DCM (3×). The combined organic phases were dried over anhydrous Na₂SO₄, filtered, and concentrated. The title compound was obtained as a white powder (210 mg, 0.871 mmol, 92%). LCMS (acidic mode) Rₜ 3.69 min, purity >99% [M+H]⁺ calculated 242.01, found 241.95. ¹H NMR (500 MHz, DMSO-d₆) δ 13.22 (s, 1H), 8.60 (s, 1H), 8.28 (ddd, J=8.8, 8.8, 6.5 Hz, 1H), 7.56 (ddd, J=11.6, 9.1, 2.5 Hz, 1H), 7.32 (ddd, J=8.5, 8.5, 2.6 Hz, 1H).

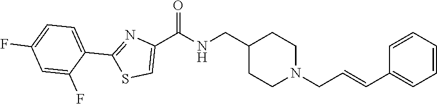 [SMILES: O=C(NCC1CCN(C/C=C/c2ccccc2)CC1)c1csc(-c2ccc(F)cc2F)n1]

### N-((1-cinnamylpiperidin-4-yl)methyl)-2-(2,4-difluorophenyl)thiazole-4-carboxamide (82)

The title compound was synthesised according to general procedure E. Acid 81, (1-cinnamylpiperidin-4-yl)methanamine hydrochloride (264 mg, 0.871 mmol, 1.0 eq.), DIPEA (0.4 mL, 2 mmol, 2.5 eq.) and HATU (497 mg, 1.31 mmol, 1.5 eq.) were used. The mixture was stirred 16 h at RT. The crude product was purified by NP column chromatography (using a linear gradient of 10-30% EtOAc:MeOH:TEA 90:5:5 in cHex to obtain the title compound as a white solid (207 mg, 52%). LCMS (acidic mode) Rₜ 3.50 min, purity >99% [M+H]⁺ calculated 454.18, found 454.20. ¹H NMR (500 MHz, Chloroform-d) δ 8.28 (ddd, J=8.7, 8.6, 6.4 Hz, 1H), 8.19 (s, 1H), 7.53 (t, J=6.5 Hz, 1H), 7.40-7.34 (m, 2H), 7.34-7.27 (m, 2H), 7.25-7.19 (m, 1H), 7.03 (dddd, J=8.7, 7.6, 2.5, 0.9 Hz, 1H), 6.98 (ddd, J=11.0, 8.5, 2.5 Hz, 1H), 6.51 (d, J=15.8 Hz, 1H), 6.30 (dt, J=15.8, 6.8 Hz, 1H), 3.41 (dd, J=6.5, 6.5 Hz, 2H), 3.17 (d, J=6.7 Hz, 2H), 3.10-2.98 (m, 2H), 2.07-1.95 (m, 2H), 1.86-1.76 (m, 2H), 1.76-1.65 (m, 1H), 1.52-1.38 (m, 2H). ¹³C NMR (126 MHz, Chloroform-d) δ 164.0 (dd, J=254.3, 12.2 Hz), 161.3, 160.5 (dd, J=255.1, 12.1 Hz), 159.9 (dd J=5.7, 1.4 Hz), 149.9, 137.0, 133.2, 130.2 (dd, J=9.9, 4.0 Hz), 128.7, 127.7, 126.5, 124.1 (d, J=8.8 Hz), 117.5 (dd, J=11.7, 3.8 Hz), 112.5 (dd, J=21.7, 3.4 Hz), 104.8 (dd, J=25.7 Hz), 61.5, 53.6, 44.9, 36.3, 30.0. HRMS m/z [M+H]⁺ C₂₅H₂₆F₂N₃OS⁺ calculated 454.1759, found 454.1762.

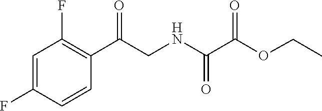 [SMILES: CCOC(=O)C(=O)NCC(=O)c1ccc(F)cc1F]

### Ethyl 2-((2-(2,4-difluorophenyl)-2-oxoethyl)amino)-2-oxoacetate (83)

Ethyl chloro oxoacetate (0.35 mL, 3.1 mmol, 1.0 eq.) was added to a suspension of 74.HCl (636 mg, 3.06 mmol, 1.0 eq.) and TEA (1.3 mL, 9.1 mmol, 3 eq.) in DCM (12 mL) at 0° C. The reaction mixture was stirred for 1 h at 0° C., then quenched with water and extracted with DCM (3×). The combined organic layers were dried over anhydrous Na₂SO₄, filtered, and concentrated in vacuo. The crude product was purified by NP column chromatography (using a linear gradient of 10-40% EtOAc in cHex) to obtain the title compound as an off-white solid. (375 mg, 45% yield). LCMS (acidic mode) Rₜ 3.68 min, purity >99% [M+H]⁺ calculated 272.07, found 272.05. ¹H NMR (500 MHz, DMSO) b 9.14 (t, J=5.8 Hz, 1H), 7.98 (ddd, J=8.7, 8.7, 6.7 Hz, 1H), 7.48 (ddd, J=11.6, 9.3, 2.5 Hz, 1H), 7.28 (ddd^{Δ}, J=8.5, 8.5, 2.4 Hz, 1H), 4.53 (dd, J=5.8, 2.8 Hz, 2H), 4.28 (q, J=7.1 Hz, 2H), 1.29 (t, J=7.1 Hz, 3H). ¹³C NMR (126 MHz, DMSO-d₆) δ 191.2 (d, J=5.1 Hz), 165.9 (dd, J=254.9, 13.0 Hz), 162.7 (dd, J=256.9, 13.4 Hz), 160.8, 157.8, 133.0 (dd, J=11.0, 4.4 Hz), 120.7 (dd, J=13.8, 3.5 Hz), 113.1 (dd, J=21.7, 3.2 Hz), 105.8 (dd, J=27.4, 26.2 Hz), 62.7, 49.6 (d, J=9.9 Hz), 14.3.

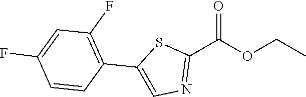 [SMILES: CCOC(=O)c1ncc(-c2ccc(F)cc2F)s1]

### Ethyl 5-(2,4-difluorophenyl)thiazole-2-carboxylate (84)

Lawesson's Reagent (400 mg, 0.990 mmol, 1.1 eq.) was added to a solution of 83 (244 mg, 0.900 mmol, 1 eq.) in 1,4-dioxane (5 mL) and the mixture was stirred at reflux for 2 h. The reaction was cooled to RT, added to water, and neutralized with a sat. aq. Na₂CO₃ solution. The aqueous layer was extracted with EtOAc (3×). The combined organic layers were dried over anhydrous Na₂SO₄, filtered, and concentrated in vacuo. The residue was purified by NP column chromatography (using a linear gradient of 0-20% EtOAc in Chex) to obtain the title compound as a light-yellow solid (200 mg, 83% yield). LCMS (acidic mode) Rₜ 4.78 min, purity 94% [M+H]⁺ calculated 270.04, found 270.00. ¹H NMR (500 MHz, DMSO) δ 8.55 (s, 1H), 8.06 (ddd, J=8.8, 8.8, 6.3 Hz, 1H), 7.53 (ddd, J=11.6, 9.1, 2.6 Hz, 1H), 7.29 (dddd, J=8.9, 8.9, 2.7, 0.9 Hz, 1H), 4.41 (q, J=7.1 Hz, 2H), 1.35 (t, J=7.1 Hz, 3H).

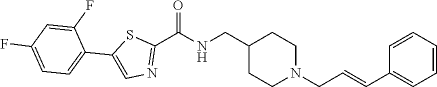 [SMILES: O=C(NCC1CCN(C/C=C/c2ccccc2)CC1)c1ncc(-c2ccc(F)cc2F)s1]

### N-((1-cinnamylpiperidin-4-yl)methyl)-5-(2,4-difluorophenyl)thiazole-2-carboxamide (85)

The title compound w67ydroxyaminsed according to general procedure G. (1-cinnamylpiperidin-4-yl)methanamine hydrochloride (270 mg, 0.891 mmol, 1.2 eq.), ester 84 (200 mg, 0.743 mmol, 1.0 eq.), 3-chlorophenol (114 mg, 0.891 mmol 1.2 eq.) and K₂CO₃ (133 mg, 0.966 mmol, 1.3 eq.) were used. The reaction mixture was heated at reflux for 16 h. The crude product was purified by NP column chromatography (using a linear gradient of 20-50% EtOAc:MeOH:TEA 90:5:5 v:v:v % in cHex) to obtain the title compound as an off-white solid (190 mg, 56% yield) LCMS (acidic mode) Rₜ 3.66 min, purity >99% [M+H]⁺ calculated 454.18, found 454.25. ¹H NMR (600 MHz, DMSO) b 8.94 (t, J=6.2 Hz, 1H), 8.41 (s, 1H), 8.00 (ddd, J=8.8, 8.8, 6.3 Hz, 1H), 7.50 (ddd, J=11.5, 9.1, 2.6 Hz, 1H), 7.43 (d^{Δ}, J=7.3 Hz, 2H), 7.31 (ta, J=7.6 Hz, 2H), 7.27 (ddd, J=8.4, 2.6 Hz, 1H), 7.25-7.19 (m, 1H), 6.52 (d, J=15.9 Hz, 1H), 6.29 (dt, J=15.9, 6.6 Hz, 1H), 3.18 (dd, J=6.5 Hz, 2H), 3.15-3.03 (m, 2H), 2.95-2.82 (m, 2H), 2.01-1.84 (m, 2H), 1.69-1.54 (m, 3H), 1.20 (dddd, J=12.1, 12.0, 12.0, 3.8 Hz, 2H). ¹³C NMR (151 MHz, DMSO) δ 163.7 (d, J=3.5 Hz), 162.4 (dd, J=250.3, 12.7 Hz), 159.3, 158.5 (dd, J=252.7, 13.8 Hz), 142.0 (d, J=3.9 Hz), 136.7, 134.9 (d, J=5.1 Hz), 130.8 (dd, J=10.0, 4.2 Hz), 128.6, 127.4, 126.2, 115.0 (dd, J=13.6, 4.0 Hz), 112.8 (dd, J=21.6, 3.4 Hz), 105.1 (dd, J=26.3, 26.3 Hz) 60.4, 52.9, 44.5, 35.6, 29.6, 26.3. HRMS m/z [M+H]⁺ C₂₅H₂₆F₂N₃OS⁺ calculated 454.1759, found 454.1759.

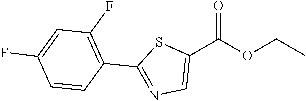 [SMILES: CCOC(=O)c1cnc(-c2ccc(F)cc2F)s1]

### Ethyl 2-(2,4-difluorophenyl)thiazole-5-carboxylate (86)

Ethyl 2-bromothiazole-5-carboxylate (700 mg, 2.97 mmol, 1.0 eq.), 2,4-difluorophenylboronic acid pinacol ester (783 mg, 3.26 mmol, 1.1 eq.), Pd(PPh3)₄ (170 mg, 0.148 mmol, 0.05 eq.) and Na₂CO₃ (628 mg, 5.93 mmol, 2.0 eq.) were mixed in 1,2-dimethoxyethane (7 mL) and water (3 mL). The mixture was irradiated in a microwave oven at 100° C. for 15 min. The solvents were evaporated and the residue was suspended in DCM and water. The aqueous layer was extracted with DCM (3×). The combined organic layers were dried, filtered and concentrated. The residue was purified by NP flash chromatography (using a linear gradient of 0-90% EtOAc in cHex) to afford the title compound as a yellow oil (315 mg, 0.98 mmol, 33%). LCMS (acidic mode) Rₜ 5.25 min, purity 96%, [M+H]⁺ calculated: 270.04, found: 270.00. ¹H NMR (500 MHz, Chloroform-d) δ 8.46 (d, J=2.3 Hz, 1H), 8.35 (ddd, J=8.7, 8.7, 6.4 Hz, 1H), 7.04 (dddd, J=8.7, 7.7, 2.5, 1.0 Hz, 1H), 6.98 (ddd, J=11.0, 8.5, 2.5 Hz, 1H), 4.40 (q, J=7.1 Hz, 2H), 1.41 (t, J=7.1 Hz, 3H).

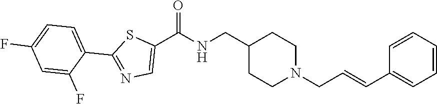 [SMILES: O=C(NCC1CCN(C/C=C/c2ccccc2)CC1)c1cnc(-c2ccc(F)cc2F)s1]

### N-((1-cinnamylpiperidin-4-yl)methyl)-2-(2,4-difluorophenyl)thiazole-5-carboxamide (87)

The title compound was synthesised according to general but required ester hydrolysis first. Ester 86 (56 mg, 0.18 mmol, 1.0 eq.) was dissolved in THE (1 mL). Aq. 2 M NaOH (0.3 mL, 0.5 mmol, 3.0 eq.) was added. The mixture was stirred at RT for 3 h. The mixture was acidified to pH 5 with 2 M aq. HCl solution and extracted with DCM (3×). The combined organic phases were dried over anhydrous Na₂SO₄, filtered, and concentrated to obtain 2-(2,4-difluorophenyl)thiazole-5-carboxylic acid as a white solid. This acid (34c, 41 mg, 0.17 mmol, 1.0 eq.) was reacted according to general procedure F using (COCl)₂ (0.02 mL, 0.2 mmol, 1.2 eq.), (1-cinnamylpiperidin-4-yl)methanamine hydrochloride (51 mg, 0.17 mmol, 1.0 eq.) and TEA (0.05 mL, 0.3 mmol, 2.0 eq.). The mixture was stirred for 3 h at RT. The crude product was purified by RP column chromatography (using a linear gradient of 0-75% ACN in water with both 0.1% FA) to obtain the title compound as a white solid (42 mg, 50%). LCMS (acidic mode) Rₜ 3.42 min, purity 97% [M+H]⁺ calculated 454.18, found 454.25. ¹H NMR (500 MHz, DMSO-d₆) δ 8.82 (t, J=5.8 Hz, 1H), 8.54 (d, J=2.3 Hz, 1H), 8.31 (dd, J=8.8, 6.5 Hz, 1H), 7.57 (ddd, J=11.6, 9.1, 2.6 Hz, 1H), 7.44 (d, J=7.5 Hz, 2H), 7.37-7.29 (m, 3H), 7.24 (t, J=7.3 Hz, 1H), 6.54 (d, J=15.9 Hz, 1H), 6.31 (dt, J=15.9, 6.6 Hz, 1H), 3.18 (dd, J=6.3, 6.3 Hz, 2H), 3.13 (d, J=6.6 Hz, 2H), 2.97-2.88 (m, 2H), 2.06-1.91 (m, 2H), 1.74-1.66 (m, 2H), 1.63-1.51 (m, 1H), 1.31-1.17 (m, 3H). ¹³C NMR (126 MHz, DMSO-d₆) δ 163.9 (dd, J=251.9, 12.6 Hz), 161.6 (d, J=5.8 Hz), 160.2, 160.1 (dd, J=253.4, 12.7 Hz), 143.3, 137.10, 136.7 (d, J=7.6 Hz), 32.5 #, 130.6 (dd, J=10.3, 3.9 Hz), 129.1, 127.9, 126.7, 117.8 (dd, J=11.8, 3.7 Hz), 113.5 (dd, J=21.9, 3.3 Hz), 105.6 (dd, J=26.3, 26.3 Hz), 60.8, 53.3, 45.2, 36.1, 30.1. HRMS m/z [M+H]⁺ C₂₅H₂₆F₂N₃OS⁺ calculated 454.1759, found 454.1757.

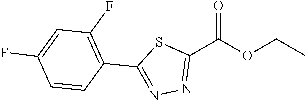 [SMILES: CCOC(=O)c1nnc(-c2ccc(F)cc2F)s1]

### Ethyl 5-(2,4-Difluorophenyl)-1,3,4-thiadiazole-2-carboxylate (88)

Ethyl 5-bromo-1,3,4-thiadiazole-2-carboxylate (1.03 g, 4.35 mmol, 1.0 eq.) and 2,4-difluorophenylboronic acid (824 mg, 5.22 mmol, 1.2 eq.) were dissolved in a mixture of NMM (956 μL, 8.70 mmol, 2.0 eq.), PhMe (8.2 mL) and H₂O (4.0 mL). Pd(OAc)₂ (9.8 mg, 0.044 mmol, 1.0 mol %) and Xantphos (25.2 mg, 0.047 mmol, 1.0 mol %) were added. The mixture was reacted for 16 h at RT. An extraction with EtOAc was performed. The organic layer was dried over Na₂SO₄, filtered and concentrated in vacuo. The residue was purified by flash chromatography (using a linear gradient of 0-90% EtOAc in cHex) to give the title compound as off-white crystalline needles (1.23 g, 4.20 mmol, 97%). LCMS (acidic mode) Rₜ 4.48 min, purity >93%, [M+H]⁺ calculated 271.03, found 271.00. ¹H NMR (500 MHz, Methanol-d4) δ 8.47 (ddd, J=8.6, 8.6, 6.3 Hz, 1H), 7.31 (ddd, J=11.3, 8.8, 2.5 Hz, 1H), 7.27-7.20 (m, 1H), 4.52 (q, J=7.1 Hz, 2H), 1.46 (t, J=7.1 Hz, 3H).

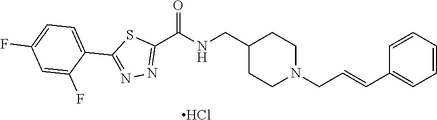 [SMILES: O=C(NCC1CCN(C/C=C/c2ccccc2)CC1)c1nnc(-c2ccc(F)cc2F)s1 ; Cl]

### 5-(2,4-difluorophenyl)-N-({1-1(2E)-3-phenylprop-2-en-1-yl]piperidin-4-yl}methyl)-1,3,4-thiadiazole-2-carboxamide hydrochloride (89.HCl)

The title compound was synthesised according to general procedure G. (1-cinnamylpiperidin-4-yl)methanamine hydrochloride (0.31 g, 1.1 mmol, 1.0 eq.), ester 88 (0.32 g, 1.4 mmol, 1.2 eq.), 3-chlorophenol (74 mg, 0.57 mmol, 0.5 eq.) and K₂CO₃ (79 mg, 0.57 mmol, 0.50 eq.) were used. The mixture was heated at reflux for 1 d. To purify, a reverse phase column purification (using a linear gradient of 100% water to 100% ACN) was performed. The fractions were freeze dried and a second reverse phase column purification was performed (using a linear gradient of 90% watIACN to 40% watIACN). The pure fractions were freeze-dried overnight and HCl in dioxane (4.0 M) was added to prepare the HCl salt. The dioxane was evaporated in vacuo and the solid was dried in the oven to obtain the title compound as yellow powder (0.11 g, 0.22 mmol, 20%). LCMS (acidic mode) Rₜ 3.39 min, purity >99% [M+H]⁺ calculated 455.17, found 455.25. ¹H NMR (500 MHz, Methanol-d₄) δ 8.42 (ddd, J=8.6, 8.5, 6.3 Hz, 1H), 7.53-7.48 (m, 2H), 7.39-7.31 (m, 3H), 7.32-7.27 (m, 1H), 7.26-7.20 (m, 1H), 6.92 (d, J=15.7 Hz, 1H), 6.34 (dt, J=15.4, 7.5, 7.5 Hz, 1H), 3.91 (d, J=7.4 Hz, 2H), 3.72-3.53 (m, 2H), 3.42 (d, J=6.5 Hz, 2H), 3.16-2.87 (m, 2H), 2.16-2.07 (m, 2H), 2.06-1.97 (m, 1H), 1.76-1.48 (m, 2H). ¹³C NMR (126 MHz, Methanol-d₄) δ 165.3 (d, J=4.9 Hz), 165.2 (dd, J=254.8, 12.6 Hz), 163.6 (d, J=7.4 Hz), 160.3 (dd, J=254.3, 12.7 Hz), 158.8, 140.5, 135.3, 130.4 (dd, J=10.3, 3.4 Hz), 128.7, 128.5, 126.7, 116.1, 114.2 (dd, J=12.2, 4.0 Hz), 112.7 (dd, J=22.4, 3.4 Hz), 104.5 (dd, J=26.3, 26.3 Hz), 58.6, 51.9, 43.8, 34.1, 27.3. HRMS m/z [M+H]⁺ C₂₄H₂₅F₂N₄OS⁺ calculated 455.1712, found 455.1730.

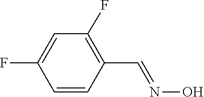 [SMILES: O/N=C/c1ccc(F)cc1F]

### 2,4-difluorobenzaldehyde oxime (89)

A mixture of TEA (0.82 mL, 5.9 mmol, 1.5 eq.) and hydroxylamine hydrochloride (411 mg, 5.91 mmol, 1.5 eq.) in water (6 mL) was added to 2,4-difluorobenzaldehyde (560 mg, 3.94 mmol, 1.0 eq.) in ethanol (6 mL) and the mixture was stirred at RT for 3 hours. The volatiles were evaporated in vacuo and the residue was dissolved in EtOAc and washed with water (3×). The organic layer was dried over anhydrous Na₂SO₄, filtered, and concentrated in vacuo to obtain the title compound as a white solid (230 mg, 37%). LCMS (acidic mode) Rₜ 3.68 min, purity >99%, [M+H]⁺ calculated 158.04, found 158.00. ¹H NMR (500 MHz, DMSO) δ 11.60 (s, 1H), 8.20 (s, 1H), 7.80 (ddd, J=8.6, 8.6, 6.8 Hz, 1H), 7.35 (ddd, J=11.6, 9.4, 2.5 Hz, 1H), 7.16 (ddd, J=8.6, 8.6, 2.4 Hz, 1H).

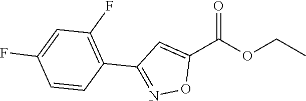 [SMILES: CCOC(=O)c1cc(-c2ccc(F)cc2F)no1]

### Ethyl 3-(2,4-difluorophenyl)isoxazole-5-carboxylate (90)

Pyridine (0.21 mL, 2.6 mmol) was added to oxime 89 (320 mg, 2.04 mmol, 1.0 eq.) in THE (4 mL). The mixture was heated to 60° C. and N-chlorosuccimide (300 mg, 2.24 mmol, 1.1 eq.) was added. The reaction mixture was stirred at 60° C. for 45 min. TEA (0.34 mL, 2.44 mmol, 1.2 eq.) and ethyl propiolate (0.23 mL, 2.2 mmol, 1.1 eq.) were added and the reaction mixture was stirred for 16 h at 60° C. The mixture was cooled to RT and concentrated in vacuo. The residue was dissolved in DCM (20 mL) and washed with aq. 1 M HCl (10 mL) and water (10 mL). The organic layer was dried over anhydrous Na₂SO₄, filtered, and concentrated in vacuo. The crude product was purified by NP chromatography (using a linear gradient of 0-10% EtOAc in cHex) to obtain the title compound as a white solid (220 mg, 43%). LCMS (acidic mode) Rₜ 4.89 min, purity 89%, [M+H]⁺ calculated 254.06, found 254.00. ¹H NMR (500 MHz, CDC₃) δ 8.04 (ddd, J=8.5, 8.4, 6.6 Hz, 1H), 7.34 (d, J=3.6 Hz, 1H), 7.04-6.93 (m, 3H), 4.47 (q, J=7.1 Hz, 2H), 1.44 (t, J=7.1 Hz, 3H).

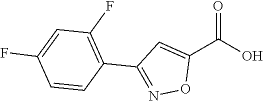 [SMILES: O=C(O)c1cc(-c2ccc(F)cc2F)no1]

### 3-(2,4-difluorophenyl)isoxazole-5-carboxylic acid (91)

Ester 90 (200 mg, 0.790 mmol, 1.0 eq.) was dissolved in ethanol and 2M aq. NaOH solution (1.0 mL, 2.0 mmol, 2.5 eq.) was added. The mixture was stirred at RT for 3 hours. EtOH was removed in vacuo and the residue was acidified to pH 5 using 1M aq. HCl solution. The precipitated solid was filtered to obtain the title compound as a white solid (90 mg, 51%). LCMS (acidic mode) Rₜ 3.50 min, purity >98%, [M+H]⁺ calculated 226.03, not detected. ¹H NMR (300 MHz, MeOD) δ 7.18 (dd^{Δ}, J=8.1, 7.7 Hz, 1H), 6.39-6.22 (m, 3H).

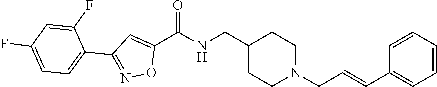 [SMILES: O=C(NCC1CCN(C/C=C/c2ccccc2)CC1)c1cc(-c2ccc(F)cc2F)no1]

### N-((1-cinnamylpiperidin-4-yl)methyl)-3-(2,4-difluorophenyl)isoxazole-5-carboxamide (92)

The title compound was synthesis according to general procedure F. Acid 91 (80 mg, 0.36 mmol, 1.0 eq.), (COCl)₂ (36 μL, 0.415 mmol, 1.2 eq.), (1-cinnamylpiperidin-4-yl)methanamine hydrochloride (119 mg, 0.391 mmol, 1.1 eq.) and TEA (0.11 mL, 0.78 mmol, 2.2 eq.) were used. The reaction mixture was stirred for 3 hours at RT. The crude product was purified by NP column chromatography (using a linear gradient of 20-60% EtOAc:MeOH:TEA 90:5:5 v:v:v % in Chex) to obtain the title compound as a white solid (36 mg, 23%). LCMS (acidic mode) Rₜ 3.56 min, purity >95%, [M+H]⁺ calculated 438.20, found 438.25. ¹H NMR (600 MHz, DMSO) δ 9.05 (t, J=5.8 Hz, 1H), 8.01 (ddd, J=8.6, 8.6, 6.5 Hz, 1H), 7.52 (ddd, J=11.5, 9.3, 2.5 Hz, 1H), 7.47 (d, J=2.6 Hz, 1H), 7.43 (d^{Δ}, J=7.3 Hz, 2H), 7.33-7.27 (m, 3H), 7.22 (ta, J=7.3 Hz, 1H), 6.51 (d, J=15.9 Hz, 1H), 6.29 (dt, J=15.9, 6.6 Hz, 1H), 3.17 (dd, J=6.3, 6.3 Hz, 2H), 3.11-3.05 (m, 2H), 2.88 (d^{Δ}, J=10.5 Hz, 2H), 1.95-1.86 (m, 2H), 1.66 (d^{Δ}, J=12.0 Hz, 2H), 1.60-1.52 (m, 1H), 1.20 (dddd, J=12.7, 12.5, 12.5, 3.5 Hz, 2H). ¹³C NMR (151 MHz, DMSO) δ 164.2, 161.9 (dd, J=251.1, 12.2 Hz), 159.9 (dd, J=253.6, 13.7 Hz), 157.4, 155.4, 136.7, 131.4 #, 130.9 (dd, J=9.9, 4.0 Hz), 128.6, 128.0^{#}, 127.3, 126.2, 112.8 (dd, J=21.9, 3.6 Hz), 112.6 (d, J=8.9 Hz), 105.8 (d, J=6.7 Hz), 105.3 (dd, J=25.9, 25.9 Hz), 60.5, 52.9, 44.4, 35.5, 29.7. HRMS m/z [M+H]⁺ C₂₅H₂₆F₂N₃O₂⁺ calculated 438.1988, found 438.1979.

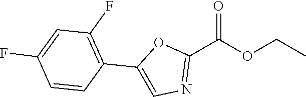 [SMILES: CCOC(=O)c1ncc(-c2ccc(F)cc2F)o1]

### Ethyl 5-(2,4-difluorophenyl)oxazole-2-carboxylate (93)

POCl₃ (0.33 mL, 3.6 mmol) was added dropwise to a solution of 83 (310 mg, 1.14 mmol), in toluene (5 mL). The mixture was stirred at 110° C. for 16 h. After cooling the solution to 0° C., it was quenched by dropwise addition of water (2 mL) and neutralized with sat. aq. NaHCO_{3.} The aqueous layer was extracted with DCM (3×) and the combined organic layers were dried over anhydrous Na₂SO₄, filtered, and concentrated in vacuo. The residue was purified by NP column chromatography (using a linear gradient of 0-20% EtOAc in cHex) to obtain the title compound as a white solid (170 mg, 59%). LCMS (acidic mode) Rₜ 4.53 min, purity >99% [M+H]⁺ calculated 254.06, found 254.05. ¹H NMR (500 MHz, DMSO-d₆) δ 7.90 (ddd, J=8.7, 8.7, 6.3 Hz, 1H), 7.80 (d, J=3.4 Hz, 1H), 7.55 (ddd, J=11.5, 9.2, 2.5 Hz, 1H), 7.37-7.26 (m, 1H), 4.41 (q, J=7.1 Hz, 2H), 1.35 (t, J=7.1 Hz, 3H).

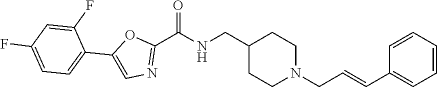 [SMILES: O=C(NCC1CCN(C/C=C/c2ccccc2)CC1)c1ncc(-c2ccc(F)cc2F)o1]

### N-((1-cinnamylpiperidin-4-yl)methyl)-5-(2,4-difluorophenyl)oxazole-2-carboxamide (94)

The title compound was synthesized according to general procedure G. (1-cinnamylpiperidin-4-yl)methanamine hydrochloride (244 mg, 0.806 mmol, 1.2 eq.), ester 93 (170 mg, 0.671 mmol, 1 eq.), 3-chlorophenol (103 mg, 0.806 mmol 1.2 eq.) and K₂CO₃ (195 mg, 1.410 mmol) were used. The reaction mixture was heated at reflux for 16 h. The crude product was purified by NP column chromatography (using a linear gradient of 20-50% EtOAc:MeOH:TEA 90:5:5 v:v:v % in cHex) to obtain the title compound as a light pink solid (140 mg, 44% yield) LCMS (acidic mode) Rₜ 3.51 min, purity >95% [M+H]⁺ calculated 438.20, found 438.25. ¹H NMR (500 MHz, DMSO) δ 9.05 (t, J=6.1 Hz, 1H), 7.97 (ddd, J=8.7, 8.7, 6.4 Hz, 1H), 7.69 (d, J=3.5 Hz, 1H), 7.52 (ddd, J=11.5, 9.2, 2.5 Hz, 1H), 7.45-7.40 (m, 2H), 7.34-7.28 (m, 3H), 7.24-7.19 (m, 1H), 6.50 (d, J=15.9 Hz, 1H), 6.29 (dt, J=16.0, 6.5 Hz, 1H), 3.17 (dd, J=6.5, 6.5 Hz, 2H), 3.06 (dd, J=6.6, 1.5 Hz, 2H), 2.87 (ddd, J=11.7, 3.5, 3.5 Hz, 2H), 1.89 (ddd, J=11.7, 11.6, 2.4 Hz, 2H), 1.67-1.61 (m, 2H), 1.61-1.52 (m, 1H), 1.25-1.14 (m, 3H). ¹³C NMR (126 MHz, DMSO) δ 162.6 (dd, J=250.1, 13.2 Hz), 158.7 (dd, J=253.4, 12.2 Hz), 154.5, 154.3, 146.1, 136.7, 131.6, 128.6, 128.3 (dd, J=10.2, 3.8 Hz), 127.6, 127.3, 126.1, 112.8 (dd, J=22.0, 3.0 Hz), 111.9 (dd, J=13.2, 3.5 Hz), 105.1 (dd, J=25.9, 25.9 Hz), 60.5, 53.0, 44.5, 35.6, 29.7. HRMS m/z [M+H]⁺ C₂₅H₂₆F₂N₃O₂⁺ calculated 438.1988, found 438.1987.

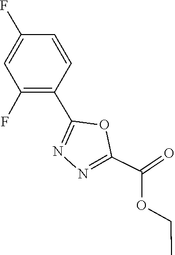 [SMILES: CCOC(=O)c1nnc(-c2ccc(F)cc2F)o1]

### 5-(2,4-difluoro-phenyl)-[1,3,4]oxadiazole-2-carboxylic acid ethyl ester (95)

To an ice-cold solution of 2,4-difluorobenzoic acid, hydrazide (0.25 g, 1.5 mmol 1.0 eq.) and TEA (0.91 mL, 6.5 mmol, 4.5 eq.) in DCM (5 mL) was dropwise added ethyl chlorooxoacetate (0.23 mL, 2.0 mmol 1.4 eq.). The resulting mixture was stirred for 2 h at 0° C. Upon completion of the starting material, toluene-4-sulfonyl chloride (0.42 g, 2.2 mmol 1.5 eq.) was added. The mixture was reacted for 16 h at RT. The mixture was extracted with satd. aq. NaHCO₃ and the aqueous layer was extracted DCM (2×). The combined organic layers were dried over anhydrous Na₂SO₄, filtered, and concentrated in vacuo. The residue was purified by flash chromatography (using a linear gradient of 0-40% EtOAc in cHex) to give the title compound as a white solid (40 mg, 0.16 mmol, 11%). LCMS (acidic mode) Rₜ 4.01 min, purity >90%, [M+H]⁺ calculated 255.06, found 255.00. ¹H NMR (500 MHz, Methanol-d₄) δ 8.18 (ddd, J=8.8, 8.0, 6.2 Hz, 1H), 7.29 (ddd, J=11.1, 9.0, 2.5 Hz, 1H), 7.26-7.20 (m, 1H), 4.52 (q, J=7.1 Hz, 2H), 1.45 (t, J=7.1 Hz, 3H).

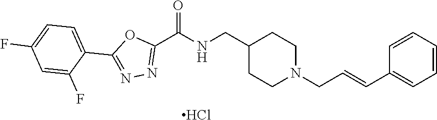 [SMILES: O=C(NCC1CCN(C/C=C/c2ccccc2)CC1)c1nnc(-c2ccc(F)cc2F)o1 ; Cl]

### 5-(2,4-difluorophenyl)-N-({1-1(2E)-3-phenylprop-2-en-1-yl]piperidin-4-yl}methyl)-1,3,4-oxadiazole-2-carboxamide hydrochloride (96.HCl)

The title compound was synthesised according to general procedure G. (1-cinnamylpiperidin-4-yl)methanamine hydrochloride (0.32 g, 1.4 mmol, 1.4 eq.), 95 (0.32 g, 1.2 mmol, 1.0 eq.), 3-chlorophenol (64 mg, 0.50 mmol, 0.50 eq.) and K₂CO₃ (69 mg, 0.50 mmol, 0.50 eq.) were used. The mixture was heated at reflux for 1 d. The mixture was poured into ice-cold satd. aq. Na₂CO₃ and an extraction with DCM and water was performed. The organic layer was dried with Na₂SO₄, filtered and concentrated in vacuo. A reverse phase column purification (using a linear gradient of 100% water to 100% ACN) was performed. A second reverse phase column purification (using a linear gradient of 90% watIACN to 40% watIACN) was performed. The pure fractions were freeze-dried overnight and HCl in dioxane (4.0 M) was added to afford the HCl salt. The dioxane was evaporated in vacuo and the solid was dried in the oven to obtain the title compound as an off-white powder (50 mg, 0.11 mmol, 11%). LCMS (acidic mode) Rₜ 3.14 min, purity >88% [M+H]⁺ calculated 439.19, found 439.20. ¹H NMR (500 MHz, Methanol-d₄) δ 8.24-8.15 (m, 1H), 7.56-7.45 (m, 2H), 7.40-7.28 (m, 4H), 7.28-7.22 (m, 1H), 6.92 (d, J=15.8 Hz, 1H), 6.43-6.27 (m, 1H), 3.91 (d, J=7.5 Hz, 1H), 3.69-3.62 (m, 2H), 3.40 (d, J=6.6 Hz, 2H), 3.07-2.93 (m, 2H), 2.15-2.04 (m, 2H), 2.05-1.98 (m, 1H), 1.68-1.40 (m, 2H). ¹³C NMR (126 MHz, Methanol-d₄) δ 167.1 (dd, J=255.6, 11.9 Hz), 163.4 (d, J=5.9 Hz), 162.5 (dd, J=261.2, 12.7 Hz), 159.9, 155.6, 142.0, 136.7, 133.0 (dd, J=10.7, 2.3 Hz), 130.1, 129.9, 128.1, 117.5, 113.9 (dd, J=22.6, 3.6 Hz), 109.5 (dd, J=12.2, 3.9 Hz), 106.6 (dd, J=26.3, 24.9 Hz), 60.0, 53.2, 45.2, 35.4, 28.6. HRMS m/z [M+H]⁺ C₂₄H₂₅F₂N₄O₂⁺ calculated 439.1940, found 439.1962.

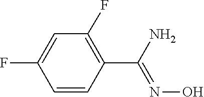 [SMILES: N/C(=N\O)c1ccc(F)cc1F]

### 2,4-difluor′-N′-hydroxybenzimidamide (97)

2,4-difluorobenzonitrile (1.2 g, 8.6 mmol, 1.0 eq.) was dissolved in ethanol (14 mL) and hydroxylamine (50% in water, 0.86 mL, 13 mmol, 1.5 eq.) was added. The mixture was stirred at 80° C. for 16 h. The reaction mixture was cooled to RT and the volatiles were evaporated in vacuo to obtain the title compound as a white solid (1.45 g, 98% yield). LCMS (acidic mode) Rₜ 1.06 min, purity 94%, [M+H]⁺ calculated 173.05, found 173.00. ¹H NMR (300 MHz, DMSO) δ 9.64 (s, 1H), 7.54 (ddd, J=8.5, 6.8 Hz, 1H), 7.28 (ddd, J=11.9, 9.5, 2.5 Hz, 1H), 7.10 (ddd, J=8.6, 2.5, 0.9 Hz, 1H), 5.83 (s, 2H).

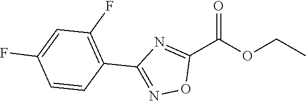 [SMILES: CCOC(=O)c1nc(-c2ccc(F)cc2F)no1]

### Ethyl 3-(2,4-difluorophenyl)-1,2,4-oxadiazole-5-carboxylate (98)

Ethyl chlorooxoacetate (2.3 mL, 22 mmol) was added dropwise to a mixture of amine 97 (1.55 g, 9.0 mmol) and pyridine (2.9 mL, 36 mmol) in DCM. The mixture was stirred at reflux for 2 hours. The reaction mixture was concentrated in vacuo and purified by NP column chromatography (using DCM) to obtain the title compound as a colorless oil (1.8 g, 79%). LCMS (acidic mode) Rₜ 4.60 min, purity 93%, [M+H]⁺ calculated 255.06, found 255.05. ¹H NMR (300 MHz, DMSO) δ 8.18-8.04 (m, 1H), 7.55 (ddd, J=10.1, 2.4 Hz, 1H), 7.40-7.26 (m, 1H), 4.47 (q, J=7.1 Hz, 2H), 1.37 (t, J=7.1 Hz, 3H).

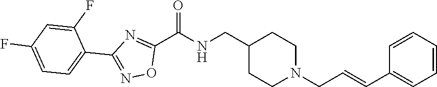 [SMILES: O=C(NCC1CCN(C/C=C/c2ccccc2)CC1)c1nc(-c2ccc(F)cc2F)no1]

### N-((1-cinnamylpiperidin-4-yl)methyl)-3-(2,4-difluorophenyl)-1,2,4-oxadiazole-5-carboxamide (99)

The title compound was synthesized according to general procedure G. (1-cinnamylpiperidin-4-yl)methanamine hydrochloride (1.07 g, 2.95 mmol, 1.2 eq.), ester 98 (750 mg, 2.95 mmol, 1.0 eq.), 3-chlorophenol (455 mg, 3.54 mmol 1.2 eq.) and K₂CO₃ (530 mg, 3.84 mmol, 1.3 eq.) were used. The reaction mixture was heated at reflux for 16 h. The crude product was purified by NP column chromatography (using a linear gradient of 20-50% EtOAc:MeOH:TEA 90:5:5 v:v:v % in cHex) to obtain the title compound as an off-white solid (720 mg, 56% yield). LCMS (acidic mode) Rₜ 3.53 min, purity >99%, [M+H]⁺ calculated 439.19, found 439.25. ¹H NMR (500 MHz, DMSO) b 9.48 (t, J=5.9 Hz, 1H), 8.12 (ddd, J=8.5, 8.5, 6.6 Hz, 1H), 7.57 (ddd, J=11.6, 11.6, 9.4, 2.5 Hz, 1H), 7.42 (d^{Δ}, J=7.3 Hz, 2H), 7.36 (ddd, J=8.4, 2.5 Hz, 1H), 7.30 (ta, J=7.6 Hz, 2H), 7.21 (tt, J=7.3, 1.0 Hz, 1H), 6.50 (d, J=15.9 Hz, 1H), 6.28 (dt, J=15.9, 6.5 Hz, 1H), 3.20 (dd, J=6.4 Hz, 2H), 3.06 (d, J=6.3 Hz, 2H), 2.87 (d^{Δ}, J=11.4 Hz, 2H), 1.93-1.85 (m, 2H), 1.66 (d^{Δ}, J=12.8 Hz, 2H), 1.63-1.54 (m, 1H), 1.20 (dddd, J=12.2, 12.1, 12.1, 3.4 Hz, 3H). ¹³C NMR (126 MHz, DMSO) b 169.0, 164.3 (dd, J=253.3, 12.7 Hz), 164.2 (d, J=5.6 Hz), 160.5 (dd, J=259.2, 13.4 Hz), 152.9, 136.7, 132.3 (dd, J=10.5, 3.5 Hz), 131.6, 128.5, 127.6, 127.3, 126.1, 112.8 (dd, J=21.9, 3.7 Hz), 110.7 (dd, J=12.4, 4.0 Hz), 105.6 (dd, J=25.7, 25.7 Hz), 60.5, 52.9, 44.8, 35.4, 29.7. HRMS m/z [M+H]⁺ C₂₄H₂₅F₂N₄O₂⁺ calculated 439.1940, found 439.1931.

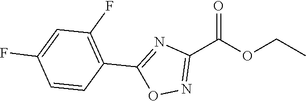 [SMILES: CCOC(=O)c1noc(-c2ccc(F)cc2F)n1]

### Ethyl 5-(2,4-difluorophenyl)-1,2,4-oxadiazole-3-carboxylate (100)

Step i) 2,4-difluorbenzoic acid (1.00 g, 6.36 mmol, 1.2 eq.) was dissolved in DCM (11 mL) and oxalyl chloride (0.64 mL, 7.4 mmol, 1.4 eq.) was added and the reaction was activated by the addition of a few drops of DMF. The mixture was stirred for 10 min at RT and the volatiles were evaporated in concentrated to obtain 2,4-difluorobenzo75ydroxyaide. Step ii) Ethyl 2-ami75ydroxyaminoino)acetate (700 mg, 5.30 mmol, 1.0 eq.) was dissolved in 2,6-dimethylpyridine (1.8 mL, 16 mmol, 3.0 eq.) and treated dropwise with a solution of 2,4-difluorobenzoyl chloride in DCM (11 mL). The reaction mixture was stirred overnight at RT. The yellow suspension was dissolved with DCM (50 mL) and washed with water (50 mL), 1 M aq. HCl solution (50 mL) and brine (50 mL). The organic layer was dried over anhydrous Na₂SO₄, filtered, and concentrated in vacuo to obtain ethyl 2-(2,4-difluorobenzamido)-75ydroxyaminoino)acetate. Step iii) ethyl 2-(2,4-difluorobenzamido)-75ydroxyaminoino)acetate was heated for 1 h at 185° C. After cooling down, the title compound was obtained as a brown solid and was continued without further purification. (778 mg, 58%). LCMS (acidic mode) Rₜ 4.46 min, purity >98%, [M+H]⁺ calculated 255.06, found 255.05. ¹H NMR (500 MHz, DMSO) δ 8.25 (dd^{Δ}, J=8.4, 8.4 Hz, 1H), 7.64 (ddd, J=9.0, 9.0, 2.1 Hz, 1H), 7.38 (ddd^{Δ}, J=8.1, 8.1, 1.7 Hz, 1H), 4.45 (q, J=7.1 Hz, 2H), 1.36 (t, J=7.1 Hz, 3H).

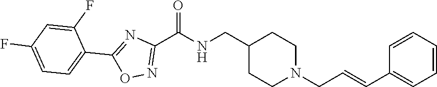 [SMILES: O=C(NCC1CCN(C/C=C/c2ccccc2)CC1)c1noc(-c2ccc(F)cc2F)n1]

### N-((1-cinnamylpiperidin-4-yl)methyl)-5-(2,4-difluorophenyl)-1,2,4-oxadiazole-3-carboxamide (101)

The title compound was synthesized according to general procedure G. (1-cinnamylpiperidin-4-yl)methanamine hydrochloride (501 mg, 1.65 mmol, 1.2 eq.), ester 100 (350 mg, 1.38 mmol, 1.0 eq.), 3-chlorophenol (212 mg, 1.65 mmol 1.2 eq.) and K₂CO₃ (247 mg, 1.79 mmol, 1.3 eq.) were used. The reaction mixture was heated at reflux for 16 h. The crude product was purified by NP column chromatography (using a linear gradient of 40% EtOAc:MeOH:TEA 90:5:5 v:v:v % in cHex) to obtain the title compound as an off-white solid (67 mg, 11% yield). LCMS (acidic mode) Rₜ 3.42 min, purity >96%, [M+H]⁺ calculated 439.19, found 439.25. ¹H NMR (500 MHz, DMSO) b 9.07 (t, J=5.9 Hz, 1H), 8.26 (ddd, J=8.5, 6.5 Hz, 1H), 7.65 (ddd, J=11.5, 9.4, 2.4 Hz, 1H), 7.46-7.38 (m, 3H), 7.31 (ta, J=7.6 Hz, 2H), 7.22 (ta, J=7.3 Hz, 1H), 6.50 (d, J=16.0 Hz, 1H), 6.29 (dt, J=15.9, 6.5 Hz, 1H), 3.19 (dd, J=6.4 Hz, 2H), 3.06 (d, J=6.3 Hz, 2H), 2.87 (d^{Δ}, J=11.4 Hz, 2H), 1.89 (dd^{Δ}, J=10.7 Hz, 2H), 1.65 (d^{Δ}, J=12.8 Hz, 2H), 1.62-1.53 (m, 1H), 1.21 (dddd^{Δ}, J=12.1, 5.3 Hz, 2H). ¹¹C NMR (126 MHz, DMSO) δ 172.2 (d, J=4.4 Hz), 165.5 (dd, J=255.7, 12.4 Hz), 164.0, 160.8 (dd, J=260.9, 13.3 Hz), 156.1, 136.7, 132.9 (d, J=10.8 Hz), 131.6, 128.5, 127.6, 127.3, 126.1, 113.3 (dd, J=22.4, 3.5 Hz), 108.6 (dd, J=11.8, 3.7 Hz), 106.0 (dd, J=25.8, 25.8 Hz), 60.5, 52.9, 44.5, 35.5, 29.7. HRMS m/z [M+H]⁺ C₂₄H₂₅F₂N₄O₂⁺ calculated 439.1940, found 439.1923.

### Appendix 1 Pharmacological Data

TABLE 1
Binding study

| Molecule # | Affinity |
|---|---|
| 1 | ++++ |
| 2 | + |
| 7 | + |
| 8 | + |
| 9 | + |
| 12 | ++ |
| 14 | + |
| 15 | +++ |
| 16 | +++ |
| 17 | +++ |
| 18 | +++ |
| 19 | +++ |
| 20 | +++ |
| 22 | ++ |
| 23 | +++ |
| 24 | ++++ |
| 25 | ++++ |
| 26 | ++++ |
| 27 | ++++ |
| 28 | +++ |
| 29 | +++ |
| 30 | ++ |
| 31 | ++ |
| 32 | + |
| 33 | ++++ |
| 34 | ++++ |
| 35 | + |
| 36 | ++++ |
| 37 | ++++ |
| 38 | + |
| 39 | ++++ |
| 40 | ++ |
| 41 | ++ |
| 42 | ++++ |
| 43 | + |
| 44 | ++++ |
| 45 | ++++ |
| 46 | ++++ |
| 47 | +++ |
| 48 | ++ |
| 49 | +++ |
| 50 | ++++ |
| 51 | + |
| 52 | +++ |
| 53 | +++ |
| 58 | ++ |
| 61 | ++++ |
| 64 | +++ |
| 68 | ++ |
| 72 | +++ |
| 77 | ++ |
| 80 | ++ |
| 82 | ++ |
| 85 | ++++ |
| 87 | ++++ |
| 89 | ++++ |
| 92 | ++++ |
| 94 | +++ |
| 96 | ++++ |
| 99 | +++ |

Rating
pKᵢ < 5 = +
pKᵢ 5-6.0 = ++,
pKᵢ 6.0-7.0 = +++,
pKᵢ ≥ 7.0 = ++++

### Pharmacokinetic Profiles—Metabolic Stability—Target Engagement Studies

Additional studies have been performed using compounds 1 and 44, and compound ACT-1004-1239 (CAS n° 2178049-58-4, described in WO2018/019929), which is used as a reference compound for comparison.

### Single-Dose Pharmacokinetics Study in Mice.

Male CD1 mice were administered IP with compound dissolved in vehicle (10% HP-β-CD in 100 mM Citrate buffer, pH 7) at a dose of 30 mg/kg and mice were sacrificed at different time points (n=3 per time point) following compound administration. Blood samples were harvested at different time points and compound concentration was determined by LC-MS/MS. The results are shown in Table 2.

### In Vitro Microsomal Stability Assay to Measure Metabolic Stability of Compounds.

Mouse liver microsomes (from S9 liver fraction from male CD-1 mice) are co-incubated with an NADPH regenerative system (1.3 mM NADP+, 3.3. mM glucose-6-phoase, 3.3 mM MgCl₂ and 0.4 units/ml glucose-6-phosphate-dehydrogenase) and 10 μM compound for 15, 30 or 60 minutes. Reaction was terminated by centrifugation at max speed and samples were analyzed by LC-MS. The results are shown in Table 2.

### Target Engagement Study in Mice.

Male CD1 mice were administered IP with compound dissolved in vehicle (10% HP-β-CD in 100 mM Citrate buffer, pH 7) at a dose of 30 mg/kg and mice were sacrificed 16 h (n=3 per time point) following compound administration. Blood samples were harvested and CXCL12 concentration was determined by CXCL12 ELISA kit.

The results are shown in Table 2.

TABLE 2

| | | Metabolic | Target |
|---|---|---|---|
| | | stability | engagement |
| | | half-life | (CXCL12 |
| | PK half-life | <2 h = +, | fold-increase) |
| | <2 h = +, | 2-4 h = ++, | <1 = −, |
| | 2-4 h = ++, | 4-10 h = +++, | 1-3 = +, |
| Molecule # | >4 h = +++ | >10 h = ++++ | >3 = ++ |
| Reference (ACT- | + | ++ | ++ |
| 1004-1239) | | | |
| 1 | ++ | ++++ | |
| 44 | +++ | ++ | ++ |

These results show that the pharmacokinetic profiles (half-lives) of molecules 1 and 44 according to the present disclosure are better than the reference compound (ACT-1004-1239). Moreover, the metabolic stability and target engagement of molecules 1 and 44 according to the present disclosure are comparable or better than the reference compound.

Therefore, these results show that the compounds according to the present disclosure have enhanced pharmacokinetic properties, and good metabolic stability and affinity.

1. A compound of formula (II): 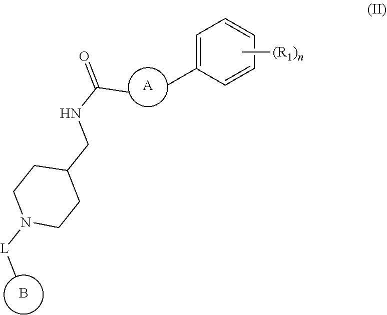
wherein
Each R₁ is independently selected from halogen;
n is an integer between 2 and 5;
Ring A is an heteroaryl having 5 ring atoms;
L is a bond or a divalent linker selected from the group consisting of —(CR₃R₄)ₚ— p being an integer between 1 and 12, C₁-C₁₂ alkylene, C₁-C₁₂ heteroalkylene, C₃-C₆ cycloalkylene, C₂-C₁₂ alkenylene, C₂-C₁₂ alkynylene, —CO—, —O—, —NR′—, —S—, —SO—, and —SO₂—, said heteroalkylene, alkenylene, and alkynylene being optionally interspersed with one or more groups selected from —(C₃-C₆ cycloalkyl)-, and said alkylene, heteroalkylene, cycloalkylene, alkenylene, and alkynylene being optionally substituted with one or more substituents;
Ring B is a ring selected from the group consisting of C₃-C₁₂ cycloalkyl, heterocyclyl having 5 to 10 ring atoms, aryl having 6 to 10 ring atoms, and heteroaryl having 5 to 10 ring atoms, said cycloalkyl, heterocyclyl, aryl and heteroaryl being optionally substituted with one or more substituents;
R₃ and R₄ are independently selected from H, C₁-C₆ alkyl, C₃-C₆ cycloalkyl, heterocyclyl having 5 to 10 ring atoms, aryl having 6 to 10 ring atoms, heteroaryl having 5 to 10 ring atoms; said alkyl, cycloalkyl, heterocyclyl, aryl and heteroaryl being optionally substituted with one or more substituents;
Or R₃ and R₄, together with the 0 atom to which they are bonded, form a C₃-C₁₂ cycloalkyl or a heterocyclyl having 5 to 10 ring atoms;
R′ is selected from H, C₁-C₆ alkyl, C₃-C₆ cycloalkyl, heterocyclyl having 5 to 10 ring atoms, aryl having 6 to 10 ring atoms, and heteroaryl having 5 to 10 ring atoms;
R″ and R″′ are independently selected from H, C₁-C₆ alkyl, C₃-C₆ cycloalkyl, heterocyclyl having 5 to 10 ring atoms, aryl having 6 to 10 ring atoms, heteroaryl having 5 to 10 ring atoms; said alkyl, cycloalkyl, heterocyclyl, aryl and heteroaryl being optionally substituted with one or more substituents;
Or R″ and R″′, together with the N atom to which they are linked, form a heterocycle having 5 to 10 ring atoms, or a heteroaryl having 5 to 10 ring atoms, said heterocyclyl, and heteroaryl being optionally substituted with one or more substituents;
or a pharmaceutically acceptable salt thereof.

2. A compound according to claim 1 or a pharmaceutically acceptable salt thereof, wherein said compound or pharmaceutically acceptable salt thereof is a modulator of ACKR3.

3. A compound according to claim 1 or a pharmaceutically acceptable salt thereof, wherein said compound or pharmaceutically acceptable salt thereof is binding to ACKR3 as measured with a displacement assay using a fluorescently labelled chemokine CXCL12.

4. A compound according to claim 1 or a pharmaceutically acceptable salt thereof, wherein ring A is a heteroaryl having 5 ring atoms, including at least 2 heteroatoms independently selected from N, O and S.

5. A compound according to claim 1 or a pharmaceutically acceptable salt thereof, wherein L is a bond or a divalent linker selected from C₁-C₁₂ alkylene, C₂-C₁₂ alkenylene, —CO—, and —SO₂—.

6. A compound according to claim 1 or a pharmaceutically acceptable salt thereof, wherein ring B is an aryl having 6 to 10 ring atoms, said aryl being optionally substituted with one or more substituents.

7. A compound according to claim 1 or a pharmaceutically acceptable salt thereof, wherein the compound is a compound of formula (VI) 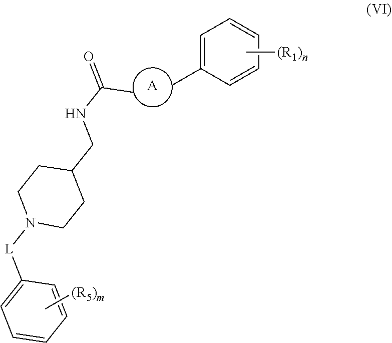
or a pharmaceutically acceptable salt thereof
wherein
m is an integer from 0 to 5; and
each R₅ is selected from halogen, —OH, —NO₂, —CN, optionally substituted C₁-C₆ alkyl, optionally substituted C₃-C₆ cycloalkyl, optionally substituted heterocyclyl having 5 to 10 ring atoms, optionally substituted aryl having 6 to 10 ring atoms, optionally substituted heteroaryl having 5 to 10 ring atoms, optionally substituted C₁-C₆ alkoxy, optionally substituted C₁-C₆ haloalkyl, C₁-C₆ haloalkoxy, —(CO)—R′, —O—(CO)—R′, —(CO)—O—R′, —(CO)—NR″R″′, —NR″—(CO)—R′, —NR″R″′, —S(O)₂—R′, and S(O)₂—NR″R″′;
R′, R″, and R″′ are as defined in claim 1.

8. A compound according to claim 1 or a pharmaceutically acceptable salt thereof, wherein the compound is a compound of formula (VII) 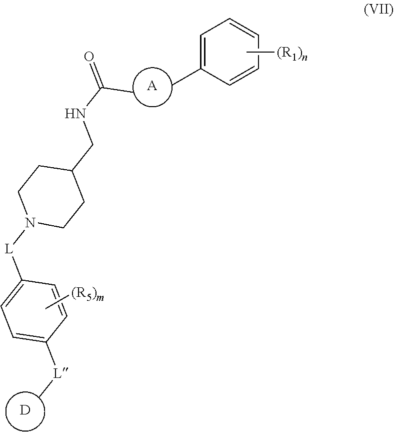
or a pharmaceutically acceptable salt thereof
Wherein
L″ is a bond or a divalent linker selected from the group consisting of —(CR₃R₄)ₚ— p being an integer between 1 and 12, C₁-C₁₂ alkylene, C₁-C₁₂ heteroalkylene, C₃-C₆ cycloalkylene, C₂-C₁₂ alkenylene, C₂-C₁₂ alkynylene, —CO—, —O—, —NR′—, —S—, —SO—, and —SO₂—, said heteroalkylene, alkenylene, and alkynylene being optionally interspersed with one or more groups selected from —(C₃-C₆ cycloalkyl)-, and said alkylene, heteroalkylene, cycloalkylene, alkenylene, and alkynylene being optionally substituted with one or more substituents;
m is an integer from 0 to 4;
each R₅ is selected from halogen, —OH, —NO₂, —CN, optionally substituted C₁-C₆ alkyl, optionally substituted C₁-C₆ alkoxy, optionally substituted C₁-C₆ haloalkyl, C₁-C₆ haloalkoxy, —(CO)—R′, —O—(CO)—R′, —(CO)—O—R′, —(CO)—NR″R″′, —NR″—(CO)—R′, —NR″R″′, —S(O)₂—R′, and S(O)₂—NR″R″′; and
Ring D is a ring selected from the group consisting of C₃-C₁₂ cycloalkyl, heterocyclyl having 5 to 10 ring atoms, aryl having 6 to 10 ring atoms, and heteroaryl having 5 to 10 ring atoms, said cycloalkyl, heterocyclyl, aryl and heteroaryl being optionally substituted with one or more substituents.

9. A compound according to claim 1, wherein the compound is selected from

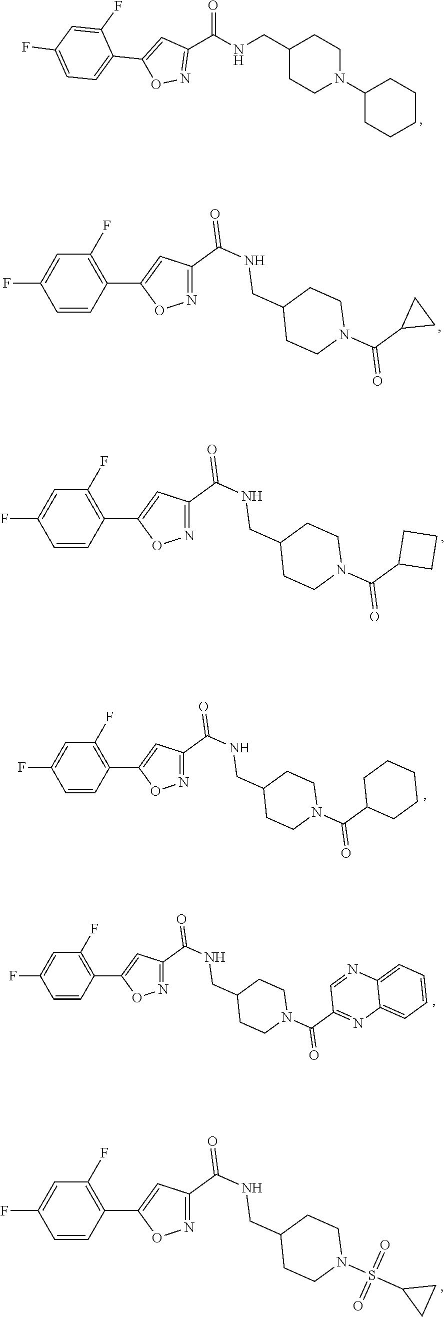 [SMILES: O=C(NCC1CCN(C2CCCCC2)CC1)c1cc(-c2ccc(F)cc2F)on1 ; O=C(NCC1CCN(C(=O)C2CC2)CC1)c1cc(-c2ccc(F)cc2F)on1 ; O=C(NCC1CCN(C(=O)C2CCC2)CC1)c1cc(-c2ccc(F)cc2F)on1 ; O=C(NCC1CCN(C(=O)C2CCCCC2)CC1)c1cc(-c2ccc(F)cc2F)on1 ; O=C(NCC1CCN(C(=O)c2cnc3ccccc3n2)CC1)c1cc(-c2ccc(F)cc2F)on1 ; O=C(NCC1CCN(S(=O)(=O)C2CC2)CC1)c1cc(-c2ccc(F)cc2F)on1]

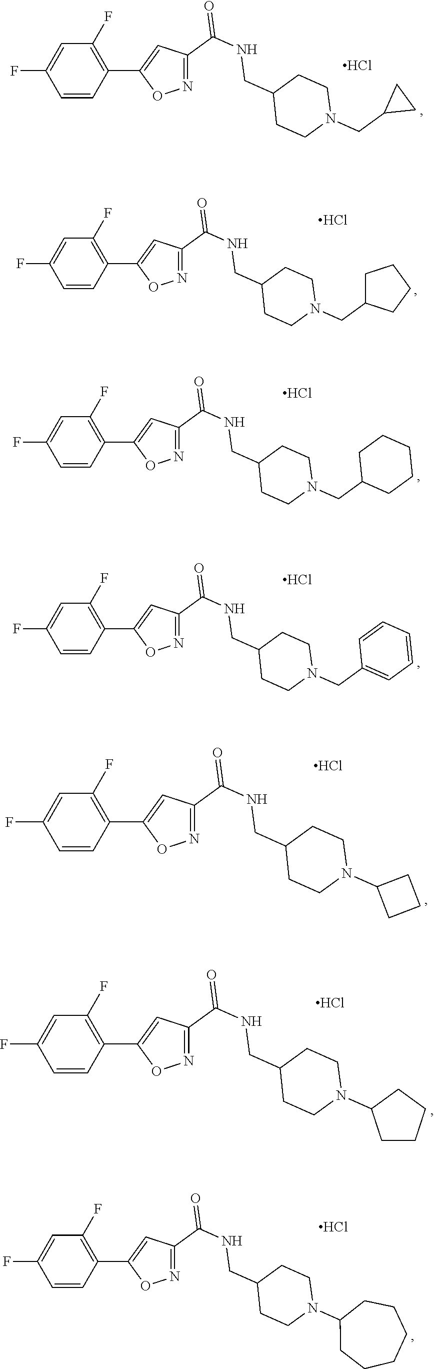

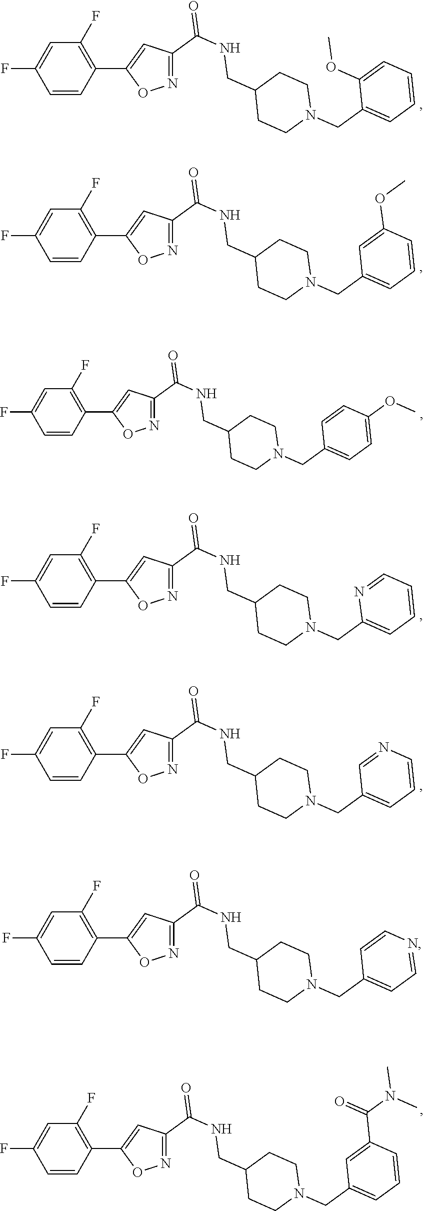

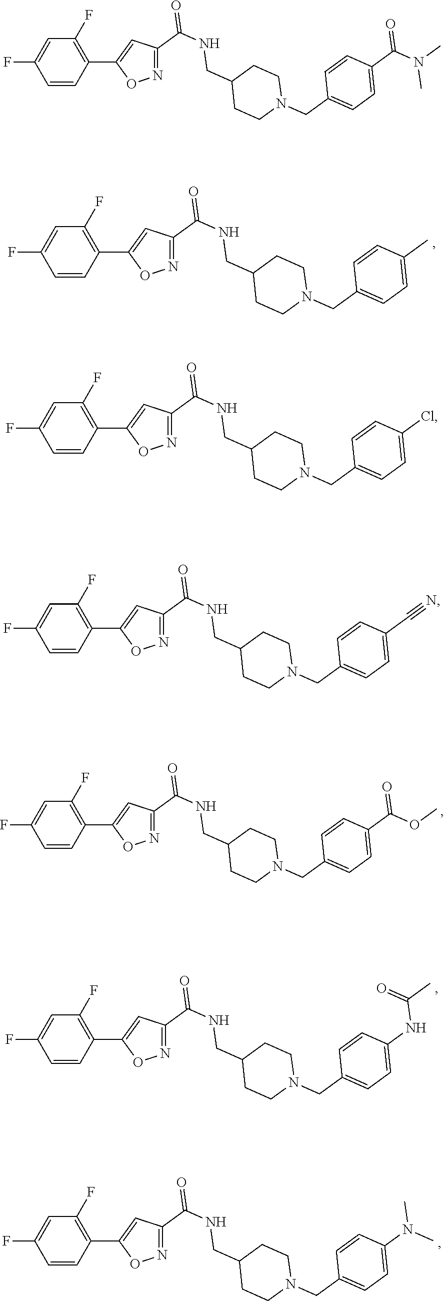

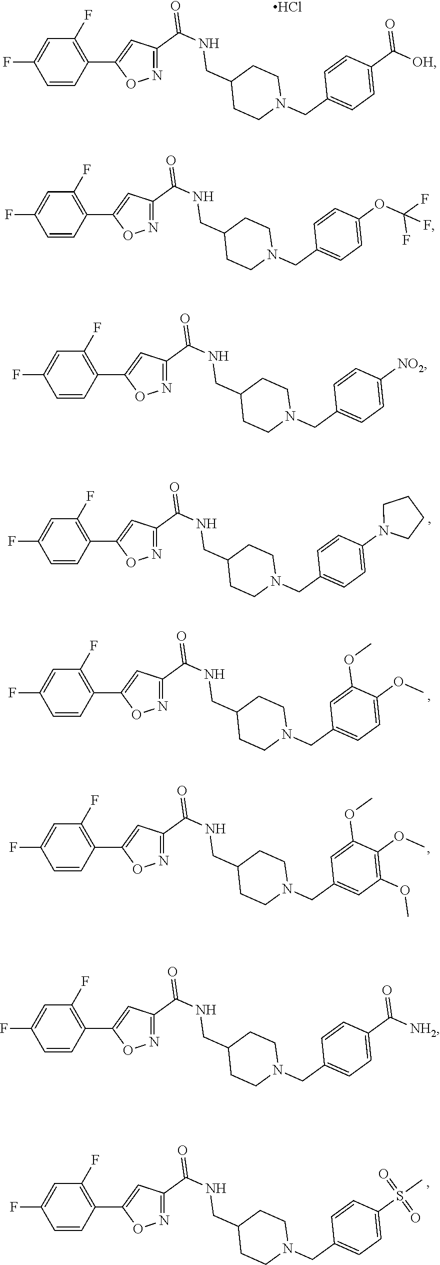

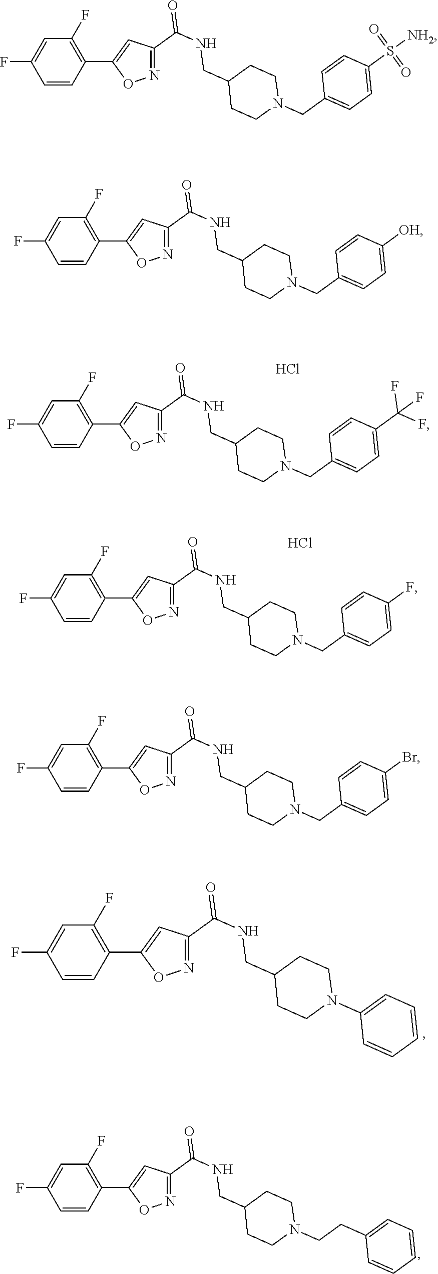

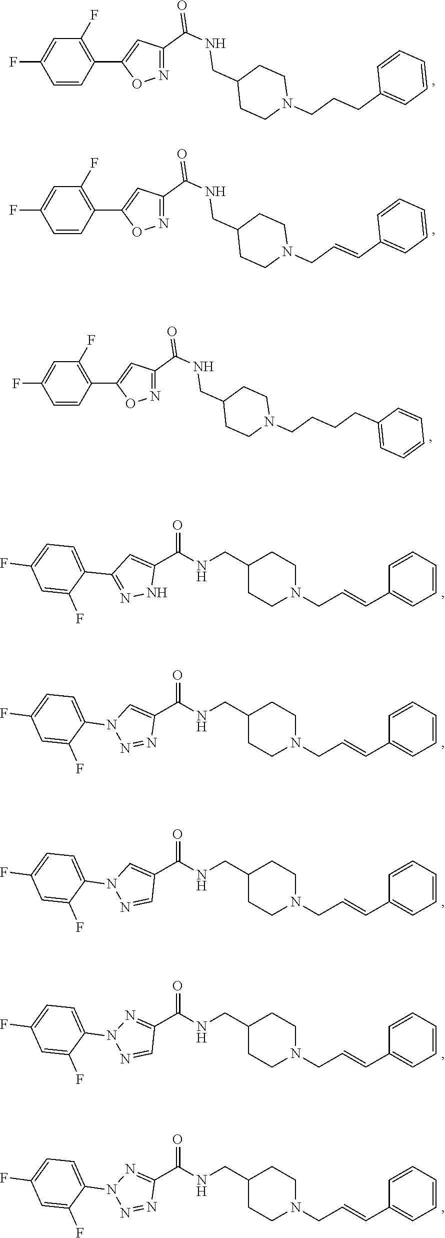

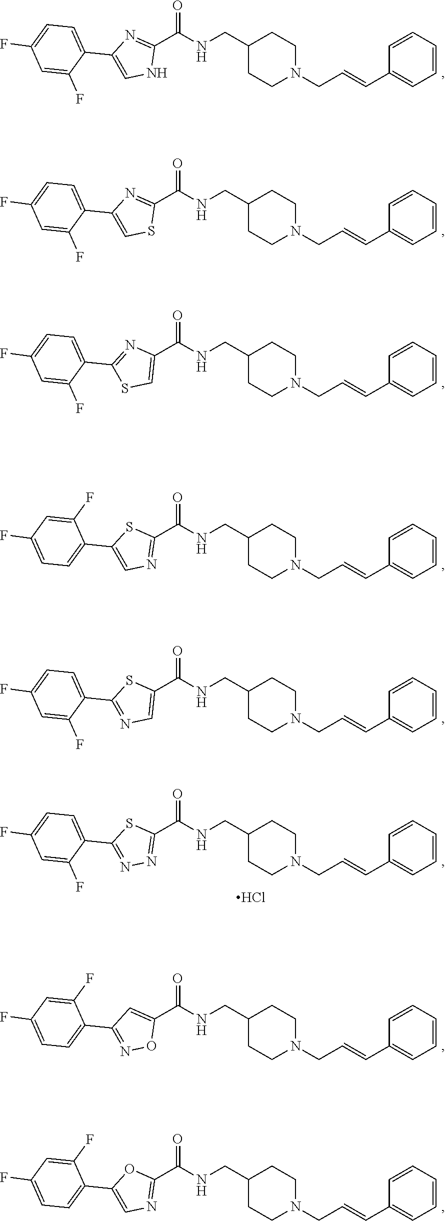

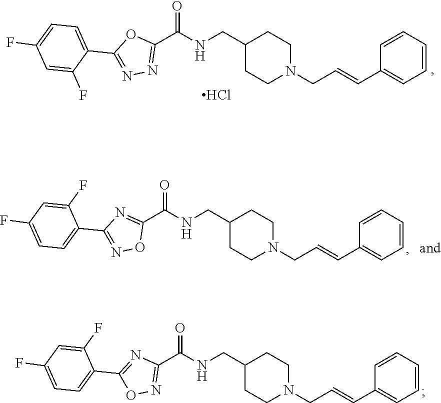 [SMILES: O=C(NCC1CCN(C/C=C/c2ccccc2)CC1)c1nnc(-c2ccc(F)cc2F)o1 ; Cl ; O=C(NCC1CCN(C/C=C/c2ccccc2)CC1)c1nc(-c2ccc(F)cc2F)no1 ; O=C(NCC1CCN(C/C=C/c2ccccc2)CC1)c1noc(-c2ccc(F)cc2F)n1]

10. A pharmaceutical composition comprising a compound according to claim 1 or a pharmaceutically acceptable salt thereof and a pharmaceutically acceptable carrier.

11-14. (canceled)

15. A compound according to claim 1, wherein the compound is selected from:

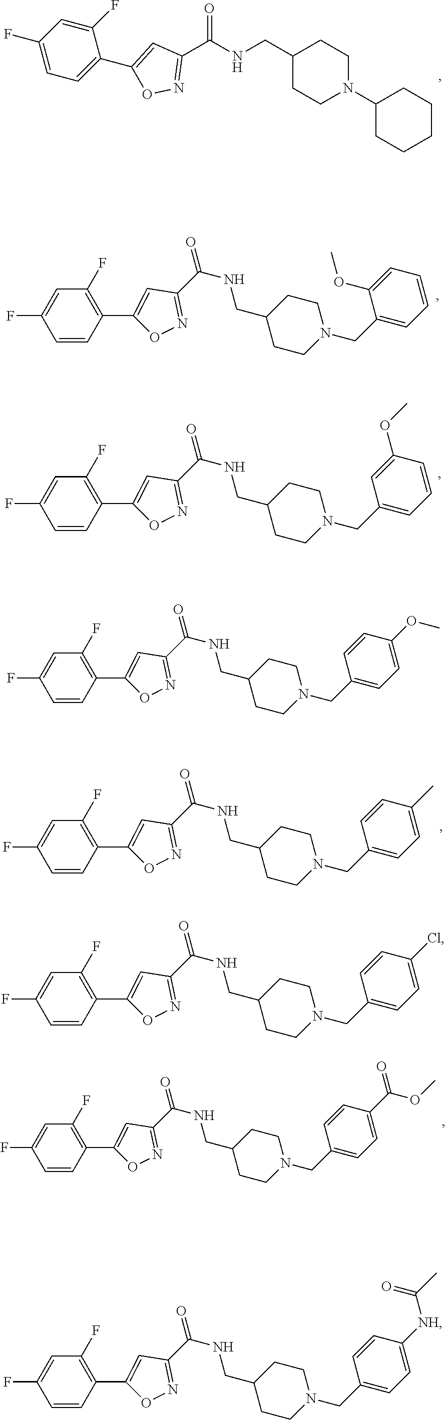

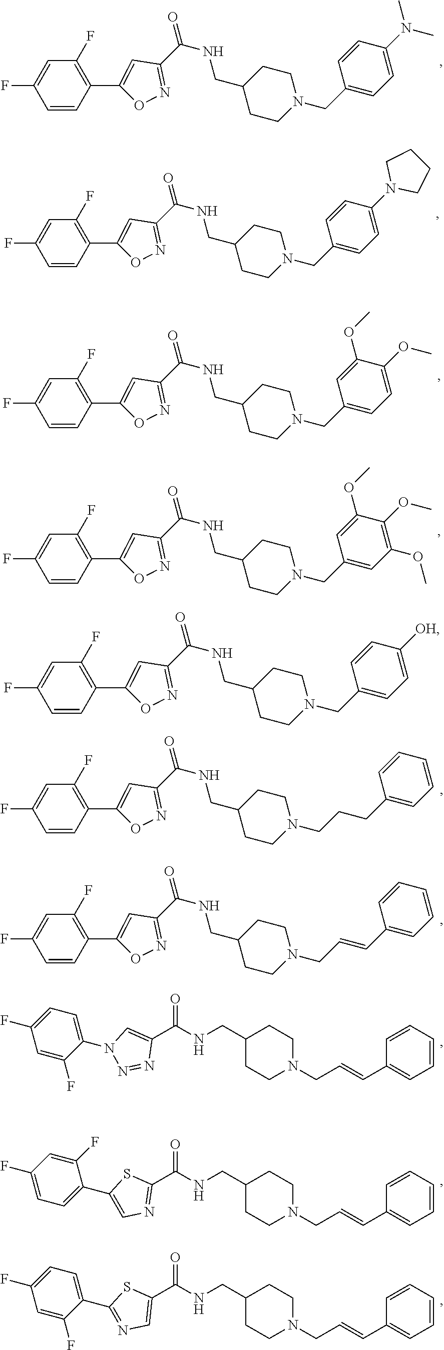

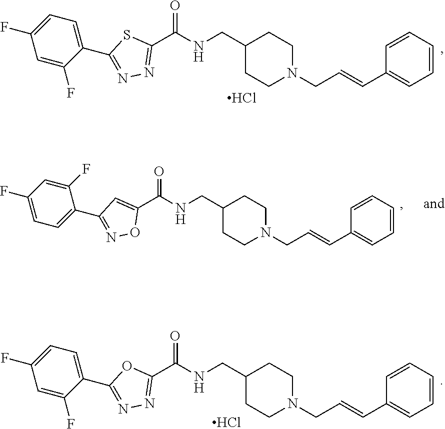 [SMILES: O=C(NCC1CCN(C/C=C/c2ccccc2)CC1)c1nnc(-c2ccc(F)cc2F)s1 ; Cl ; O=C(NCC1CCN(C/C=C/c2ccccc2)CC1)c1cc(-c2ccc(F)cc2F)no1 ; O=C(NCC1CCN(C/C=C/c2ccccc2)CC1)c1nnc(-c2ccc(F)cc2F)o1 ; Cl]

16. A pharmaceutical composition comprising a compound according to claim 9 and a pharmaceutically acceptable carrier.

17. A pharmaceutical composition comprising a compound according to claim 15 and a pharmaceutically acceptable carrier.

18. A method for treating disorders relating to the ACKR3 receptor, said method comprising administering to a subject in need thereof a therapeutically efficient amount of a compound according to claim 1 or a pharmaceutically acceptable salt thereof.

19. A method for treating cancer, an autoimmune disorder, an inflammatory disease, transplant rejection, fibrosis, or pain, said method comprising administering to a subject in need thereof a therapeutically efficient amount of a compound according to claim 1 or a pharmaceutically acceptable salt thereof.

20. The method according to claim 19, wherein the autoimmune disorder is a demyelinating disease or multiple sclerosis.

21. A method for treating cancer, an autoimmune disorder, an inflammatory disease, transplant rejection, fibrosis, or pain, said method comprising administering to a subject in need thereof a therapeutically efficient amount of a compound according to claim 9.

22. The method according to claim 21, wherein the autoimmune disorder is a demyelinating disease or multiple sclerosis.

23. A method for treating cancer, an autoimmune disorder, an inflammatory disease, transplant rejection, fibrosis, or pain, said method comprising administering to a subject in need thereof a therapeutically efficient amount of a compound according to claim 15.

24. The method according to claim 23, wherein the autoimmune disorder is a demyelinating disease or multiple sclerosis.
